SEQUENCE LISTING (22 sequences)

SEQ ID NO: 1 - DNA, 3147 bp, Homo Sapien
  CDS (23)...(2589): Nucleotide sequence encoding MTSP1
tcaagagcggcctcggggtaccatggggagcgatcgggcccgcaagggcggagggggcccgaaggacttcggcgcgggactcaagtacaactcccggcacgagaaagtgaatggcttggaggaaggcgtggagttcctgccagtcaacaacgtcaagaaggtggaaaagcatggcccggggcgctgggtggtgctggcagccgtgctgatcggcctcctcttggtcttgctggggatcggcttcctggtgtggcatttgcagtaccgggacgtgcgtgtccagaaggtcttcaatggctacatgaggatcacaaatgagaattttgtggatgcctacgagaactccaactccactgagtttgtaagcctggccagcaaggtgaaggacgcgctgaagctgctgtacagcggagtcccattcctgggcccctaccacaaggagtcggctgtgacggccttcagcgagggcagcgtcatcgcctactactggtctgagttcagcatcccgcagcacctggtggaggaggccgagcgcgtcatggccgaggagcgcgtagtcatgctgcccccgcgggcgcgctccctgaagtcctttgtggtcacctcagtggtggctttccccacggactccaaaacagtacagaggacccaggacaacagctgcagctttggcctgcacgcccgcggtgtggagctgatgcgcttcaccacgcccggcttccctgacagcccctaccccgctcatgcccgctgccagtgggccctgcggggggacgccgactcagtgctgagcctcaccttccgcagctttgaccttgcgtcctgcgacgagcgcggcagcgacctggtgacggtgtacaacaccctgagccccatggagccccacgccctggtgcagttgtgtggcacctaccctccctcctacaacctgaccttccactcctcccagaacgtcctgctcatcacactgataaccaacactgagcggcggcatcccggctttgaggccaccttcttccagctgcctaggatgagcagctgtggaggccgcttacgtaaagcccaggggacattcaacagcccctactacccaggccactacccacccaacattgactgcacatggaacattgaggtgcccaacaaccagcatgtgaaggtgagcttcaaattcttctacctgctggagcccggcgtgcctgcgggcacctgccccaaggactacgtggagatcaatggggagaaatactgcggagagaggtcccagttcgtcgtcaccagcaacagcaacaagatcacagttcgcttccactcagatcagtcctacaccgacaccggcttcttagctgaatacctctcctacgactccagtgacccatgcccggggcagttcacgtgccgcacggggcggtgtatccggaaggagctgcgctgtgatggctgggccgactgcaccgaccacagcgatgagctcaactgcagttgcgacgccggccaccagttcacgtgcaagaacaagttctgcaagcccctcttctgggtctgcgacagtgtgaacgactgcggagacaacagcgacgagcaggggtgcagttgtccggcccagaccttcaggtgttccaatgggaagtgcctctcgaaaagccagcagtgcaatgggaaggacgactgtggggacgggtccgacgaggcctcctgccccaaggtgaacgtcgtcacttgtaccaaacacacctaccgctgcctcaatgggctctgcttgagcaagggcaaccctgagtgtgacgggaaggaggactgtagcgacggctcagatgagaaggactgcgactgtgggctgcggtcattcacgagacaggctcgtgttgttgggggcacggatgcggatgagggcgagtggccctggcaggtaagcctgcatgctctgggccagggccacatctgcggtgcttccctcatctctcccaactggctggtctctgccgcacactgctacatcgatgacagaggattcaggtactcagaccccacgcagtggacggccttcctgggcttgcacgaccagagccagcgcagcgcccctggggtgcaggagcgcaggctcaagcgcatcatctcccaccccttcttcaatgacttcaccttcgactatgacatcgcgctgctggagctggagaaaccggcagagtacagctccatggtgcggcccatctgcctgccggacgcctcccatgtcttccctgccggcaaggccatctgggtcacgggctggggacacacccagtatggaggcactggcgcgctgatcctgcaaaagggtgagatccgcgtcatcaaccagaccacctgcgagaacctcctgccgcagcagatcacgccgcgcatgatgtgcgtgggcttcctcagcggcggcgtggactcctgccagggtgattccgggggacccctgtccagcgtggaggcggatgggcggatcttccaggccggtgtggtgagctggggagacggctgcgctcagaggaacaagccaggcgtgtacacaaggctccctctgtttcgggactggatcaaagagaacactggggtataggggccggggccacccaaatgtgtacacctgcggggccacccatcgtccaccccagtgtgcacgcctgcaggctggagactggaccgctgactgcaccagcgcccccagaacatacactgtgaactcaatctccagggctccaaatctgcctagaaaacctctcgcttcctcagcctccaaagtggagctgggaggtagaaggggaggacactggtggttctactgacccaactgggggcaaaggtttgaagacacagcctcccccgccagccccaagctgggccgaggcgcgtttgtgtatatctgcctcccctgtctgtaaggagcagcgggaacggagcttcggagcctcctcagtgaaggtggtggggctgccggatctgggctgtggggcccttgggccacgctcttgaggaagcccaggctcggaggaccctggaaaacagacgggtctgagactgaaattgttttaccagctcccagggtggacttcagtgtgtgtatttgtgtaaatgggtaaaacaatttatttctttttaaaaaaaaaaaaaaaaaaa

SEQ ID NO: 2 - protein, 855 aa, Homo Sapien [sequence omitted]

SEQ ID NO: 3 - DNA, 3147 bp, Homo Sapien; identical to SEQ ID NO: 1
  CDS (1865)...(2590): Nucleic acid sequence of protease domain of MTSP1

SEQ ID NO: 4 - protein, 241 aa, Homo Sapien [sequence omitted]

SEQ ID NO: 5 - DNA, 2293 bp, Artificial Sequence
  CDS (1)...(2259): CVSP16 Full Length cDNA
atggcccggcagctgctcctcccccttgtggtgcttgtcatcagtcccatcccaggagccttccaggactcagctctcagtcctacccaggaagaacctgaagatctggactgcgggcgccctgagccctcggcccgcatcgtggggggctcaaacgcgcagccgggcacctggccttggcaagtgagcctgcaccatggaggtggccacatctgcgggggctccctcatcgccccctcctgggtcctctccgccgctcactgtttcatgacgaatgggacgctggagcccgcggccgagtggtcggtactgctgggcgtgcactcccaggacgggcccctggacggcgcgcacacccgcgcagtggccgccatcgtggtgccggccaactacagccaagtggagctgggcgccgacctggccctgctgcgcctggcctcacccgccagcctgggccccgccgtgtggcctgtctgcctgccccgcgcctcacaccgcttcgtgcacggcaccgcctgctgggccaccggctggggagacgtccaggaggcagatcctctgcctctcccctgggtgctacaggaagtggagctaaggctgctgggcgaggccacctgtcaatgtctctacagccagcccggtcccttcaacctcactctccagatattgccagggatgctgtgtgctggctacccagggggccgcagggacacctgccagggtgactctggggggcccctggtctgtgaggaaggcggccgctggttccaggcaggaatcaccagctttggctttggctgtggacggagaaaccgccctggagttttcactgctgtggctacctatgaggcatggatacgggagcaggtgatgggttcagagcctgggcctgcctttcccacccagccccagaagacccagtcagatccccaggagcccagggaggagaactgcaccattgccctgcctgagtgcgggaaggccccgcggccaggggcctggccctgggaggcccaggtgatggtgccaggatccagaccctgccatggggcgctggtgtctgaaagctgggtcttggcacctgccagctgctttctggacccgaacagctccgacagcccaccccgcgacctcgacgcctggcgcgtgctgctgccctcgcacccgcgcgcggagcgggtggcgcgcctggtgcagcacgagaacgcttcgtgggacaacgccccggacctggcgctgctgcagctgcgcacgcccgtgaacctgagtgcggcttcgcggcccgtgtgcctaccccacccggaacactacttcctgcccgggagccgctgccgcctggcccgctggggccgcggggaacccgcgcttggcccaggcgcgctgctggaggcggagctgttaggcggctggtggtgccactgcctgtacggccgccagggggcggcagtaccgctgcccggagacccgccgcacgcgctctgccctgcctaccaggaaaaggaggaggtgggcagctgctggaatgactcgcgttggagccttttgtgccaggaggaggggacctggtttctggctggaatcagagactttcccagtggctgtctacgtccccgagccttcttccctctgcagactcatggcccatggatcagccatgtgactcggggagcctacctggaggaccagctagcctgggactggggccctgatggggaggagactgagacacagacttgtcccccacacacagagcatggtgcctgtggcctgcggctggaggctgctccagtgggggtcctgtggccctggctggcagaggtgcatgtggctggtgatcgagtctgcactgggatcctcctggccccaggctgggtcctggcagccactcactgtgtcctcaggccaggctctacaacagtgccttacattgaagtgtatctgggccgggcaggggccagctccctcccacagggccaccagatgacctcagcaccgcccctcctgtgccagatgacggaagggtcctggatcctcgtgggcatggctgttcaagggagccgggagctgtttgctgccattggtcctgaagaggcctggatctcccagacagtgggagaggccaacttcctgccccccagtggctccccacactggcccactggaggcagcaatctctgccccccagaactggccaaggcctcgggatccccgcatgcagtctacttcctgctcctgctgactctcctgatccagagctgaggggctagggtcccagcaccacttcccccttctc

SEQ ID NO: 6 - protein, 752 aa, Artificial Sequence
  CVSP16 Full Length Protein
MARQLLLPLVVLVISPIPGAFQDSALSPTQEEPEDLDCGRPEPSARIVGGSNAQPGTWPWQVSLHHGGGHICGGSLIAPSWVLSAAHCFMTNGTLEPAAEWSVLLGVHSQDGPLDGAHTRAVAAIVVPANYSQVELGADLALLRLASPASLGPAVWPVCLPRASHRFVHGTACWATGWGDVQEADPLPLPWVLQEVELRLLGEATCQCLYSQPGPFNLTLQILPGMLCAGYPGGRRDTCQGDSGGPLVCEEGGRWFQAGITSFGFGCGRRNRPGVFTAVATYEAWIREQVMGSEPGPAFPTQPQKTQSDPQEPREENCTIALPECGKAPRPGAWPWEAQVMVPGSRPCHGALVSESWVLAPASCFLDPNSSDSPPRDLDAWRVLLPSHPRAERVARLVQHENASWDNAPDLALLQLRTPVNLSAASRPVCLPHPEHYFLPGSRCRLARWGRGEPALGPGALLEAELLGGWWCHCLYGRQGAAVPLPGDPPHALCPAYQEKEEVGSCWNDSRWSLLCQEEGTWFLAGIRDFPSGCLRPRAFFPLQTHGPWISHVTRGAYLEDQLAWDWGPDGEETETQTCPPHTEHGACGLRLEAAPVGVLWPWLAEVHVAGDRVCTGILLAPGWVLAATHCVLRPGSTTVPYIEVYLGRAGASSLPQGHQMTSAPPLLCQMTEGSWILVGMAVQGSRELFAAIGPEEAWISQTVGEANFLPPSGSPHWPTGGSNLCPPELAKASGSPHAVYFLLLLTLLIQS

SEQ ID NO: 7 - DNA, 27 bp, Artificial Sequence
  Primer
ccctctgggtagccagcacacagcatc

SEQ ID NO: 8 - DNA, 26 bp, Artificial Sequence
  Primer
gccatcgtggtgccggccaactacag

SEQ ID NO: 9 - DNA, 27 bp, Artificial Sequence
  Primer
gcacacagcatccctggcaatatctgg

SEQ ID NO: 10 - DNA, 26 bp, Artificial sequence
  Primer
cggccaactacagccaagtggagctg

SEQ ID NO: 11 - DNA, 30 bp, Artificial Sequence
  Primer
atggcccggcagctgctcctcccccttgtg

SEQ ID NO: 12 - DNA, 28 bp, Artificial Sequence
  Primer
cggctcccgggcaggaagtagtgttccg

SEQ ID NO: 13 - DNA, 9276 bp, Pichia pastoris
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattccctagggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 14 - protein, 11 aa, Pichia pastoris
KRIASGVIAPK

SEQ ID NO: 15 - DNA, 30 bp, Artificial Sequence
  Primer
tgggtcttggcacctgccagctgctttctg

SEQ ID NO: 16 - DNA, 28 bp, Artificial Sequence
  Primer
gaagggggaagtggtgctgggaccctag

SEQ ID NO: 17 - DNA, 27 bp, Artificial Sequence
  Primer
ccctctgggtagccagcacacagcatc

SEQ ID NO: 18 - DNA, 26 bp, Artificial Sequence
  Primer
gccatcgtggtgccggccaactacag

SEQ ID NO: 19 - DNA, 30 bp, Artificial Sequence
  Primer
atcgtggtgccggccaactacagccaagtg

SEQ ID NO: 20 - DNA, 30 bp, Artificial Sequence
  Primer
acccatcacctgctcccgtatccatgcctc

SEQ ID NO: 21 - protein, 103 aa, Artificial Sequence
  Polypeptide
VSRLVISIRLPQHLGLRPPLALLELSSRVEPSPSALPICLHPAGIPPGASCWVLGWKEPQDRVPVAAAVSILTQRICDCLYQGILPPGTLCVLYAEGQENRCE

SEQ ID NO: 22 - protein, 37 aa, Artificial Sequence
  Polypeptide
NDSRWSLLCQEEGTWFLAGIRDFPSGCLRPRAFFPLQ

(19) United States
(12) Patent Application Publication
(10) Pub. No.: US 2005/0112579 A1
(43) Pub. Date: May 26, 2005

(54) Nucleic acid molecules encoding serine protease 16, the encoded polypeptides and methods based thereon

(75) Inventors: Edwin L. Madison, San Diego, CA (US); Edgar O. Ong, San Diego, CA (US); Jiunn- Chern Yeh, San Diego, CA (US)

Correspondence Address: FISH & RICHARDSON, PC, 12390 EL CAMINO REAL, SAN DIEGO, CA, 92130-2081, US

(73) Assignee: DENDREON SAN DIEGO LLC, a Delaware corporation, San Diego, CA (US)

(21) Appl. No.: 10/612,466

(22) Filed: Jul. 1, 2003

Related U.S. Application Data
(60) Provisional application No. 60/394,347, filed on Jul. 2, 2002.

(51) Int. Cl. C12Q 1/68; G01N 33/574; C07H 21/04; C12N 9/64; C12N 1/18; C07K 16/40

(52) U.S. Cl. 435/6; 435/69.1; 435/226; 435/320.1; 435/252.3; 435/254.23; 435/7.23; 530/388.26; 536/23.2

(57) ABSTRACT

Provided herein are serine proteases designated CVSP16. CVSP16 polypeptides exhibit protease activity as a single chain or as a multi-chain form. Methods using the polypeptides to identify compounds that modulate the protease activity thereof are provided. The polypeptides also serve as tumor markers.

### RELATED APPLICATIONS

Benefit of priority under 35 U.S.C. §119(e) is claimed to U.S. provisional application Ser. No. 60/394,347, filed Jul. 2, 2002, to Edwin L. Madison, Edgar O. Ong and Juinn-Chern Yeh, entitled “NUCLEIC ACID MOLECULES ENCODING SERINE PROTEASE 16, THE ENCODED PROTEINS AND METHODS BASED THEREON.” The subject matter of each of the provisional application and International PCT application No. Docket No. 24745-1625PC, filed on the same day herewith, entitled “NUCLEIC ACID MOLECULES ENCODING SERINE PROTEASE 16, THE ENCODED POLYPEPTIDES AND METHODS BASED THEREON”, is incorporated by reference in it entirety.

### FIELD OF INVENTION

Nucleic acid molecules that encode proteases and portions thereof, particularly protease domains are provided. Also provided are prognostic, diagnostic and therapeutic methods using the proteases and domains thereof and the encoding nucleic acid molecules.

### BACKGROUND OF THE INVENTION AND OBJECTS THEREOF

Cancer is a leading cause of death in the United States, developing in one in three Americans; one of every four Americans dies of cancer. Cancer is characterized by an increase in the number of abnormal neoplastic cells, which proliferate to form a tumor mass, the invasion of adjacent tissues by these neoplastic tumor cells, and the generation of malignant cells that metastasize via the blood or lymphatic system to regional lymph nodes and to distant sites.

Among the hallmarks of cancer is a breakdown in the communication among tumor cells and their environment. Normal cells do not divide in the absence of stimulatory signals, and cease dividing in the presence of inhibitory signals. Growth-stimulatory and growth-inhibitory signals are routinely exchanged between cells within a tissue. In a cancerous, or neoplastic, state, a cell acquires the ability to “override” these signals and to proliferate under conditions in which normal cells do not grow.

In order to proliferate, tumor cells acquire a number of distinct aberrant traits reflecting genetic alterations. The genomes of certain well-studied tumors carry several different independently altered genes, including activated oncogenes and inactivated tumor suppressor genes. Each of these genetic changes appears to be responsible for imparting some of the traits that, in the aggregate, represent the full neoplastic phenotype.

A variety of biochemical factors have been associated with different phases of metastasis. Cell surface receptors for collagen, glycoproteins such as laminin, and proteoglycans, facilitate tumor cell attachment, an important step in invasion and metastases. Attachment triggers the release of degradative enzymes which facilitate the penetration of tumor cells through tissue barriers. Once the tumor cells have entered the target tissue, specific growth factors are required for further proliferation. Tumor invasion and progression involves a complex series of events, in which tumor cells detach from the primary tumor, break down the normal tissue surrounding it, and migrate into a blood or lymphatic vessel to be carried to a distant site. Destruction and/or remodeling of normal tissue barriers is accomplished by the elaboration of specific enzymes that degrade the proteins of the extracellular matrix that make up basement membranes and stromal components of tissues.

A class of extracellular matrix degrading enzymes has been implicated in tumor invasion. Among these are the matrix metalloproteinases (MMP). For example, the production of the matrix metalloproteinase stromelysin is associated with malignant tumors with metastatic potential (see, e.g., McDonnell et al. (1990) Smnrs. in Cancer Biology 1:107-115; McDonnell et al. (1990) Cancer and Metastasis Reviews 9:309-319).

The capacity of cancer cells to metastasize and invade tissue is facilitated by degradation of the basement membrane. Several proteinase enzymes, including the MMPs, have been reported to facilitate the process of invasion of tumor cells. MMPs are reported to enhance degradation of the basement membrane, which thereby permits tumorous cells to invade tissues. For example, two major metalloproteinases having molecular weights of about 70 kDa and 92 kDa appear to enhance the ability of tumor cells to metastasize.

Serine Proteases

Serine proteases (SPs) have been implicated in neoplastic disease progression. Most serine proteases, which are either secreted enzymes or are sequestered in cytoplasmic storage organelles, have roles in blood coagulation, wound healing, digestion, immune responses and tumor invasion and metastasis. A class of cell surface proteins designated type II transmembrane serine proteases, which are membrane-anchored proteins with additional extracellular domains, has been identified. As cell surface proteins, they are positioned to play a role in intracellular signal transduction and in mediating cell surface proteolytic events. Other serine proteases can be membrane bound and function in a similar manner. Others are secreted. Many serine proteases exert their activity upon binding to cell surface receptors, and, hence act at cell surfaces. Cell surface proteolysis is a mechanism for the generation of biologically active proteins that mediate a variety of cellular functions.

Serine proteases, including secreted and transmembrane serine proteases, have been implicated in processes involved in neoplastic development and progression. While the precise role of these proteases has not been fully elaborated, serine proteases and inhibitors thereof are involved in the control of many intra- and extracellular physiological processes, including degradative actions in cancer cell invasion, metastatic spread, and neovascularization of tumors, that are involved in tumor progression. It is believed that proteases are involved in the degradation and remodeling of extracellular matrix (ECM) and contribute to tissue remodeling, and are necessary for cancer invasion and metastasis. The activity and/or expression of some proteases have been shown to correlate with tumor progression and development.

For example, a membrane-type serine protease MTSP1 (also called matriptase; see SEQ ID Nos. 1 and 2 from U.S. Pat. No. 5,972,616; and GenBank Accession No. AF118224; (1999) J. Biol. Chem. 274:18231-18236; U.S. Pat. No. 5,792,616; see, also Takeuchi (1999) Proc. Natl. Acad. Sci. U.S.A. 96:11054-1161) that is expressed in epithelial cancer and normal tissue (Takeucuhi et al. (1999) Proc. Natl. Acad. Sci. USA 96:11054-61) has been identified. Matriptase was originally identified in human breast cancer cells as a major gelatinase (see, U.S. Pat. No. 5,482,848), a type of matrix metalloproteinase (MMP). It has been proposed that it plays a role in the metastasis of breast cancer. Matriptase also is expressed in a variety of epithelial tissues with high levels of activity and/or expression in the human gastrointestinal tract and the prostate. MTSPs, designated MTSP3, MTSP4, MTSP6 have been described in published International PCT application No. WO 01/57194, based in International PCT application No. PCT/US01/03471.

Prostate-specific antigen (PSA), a kallikrein-like serine protease, degrades extracellular matrix glycoproteins fibronectin and laminin, and, has been postulated to facilitate invasion by prostate cancer cells (Webber et al. (1995) Clin. Cancer Res. 1:1089-94). Blocking PSA proteolytic activity with PSA-specific monoclonal antibodies results in a dose-dependent decrease in vitro in the invasion of the reconstituted basement membrane Matrigel by LNCaP human prostate carcinoma cells which secrete high levels of PSA.

Hepsin, a cell surface serine protease identified in hepatoma cells, is overexpressed in ovarian cancer (Tanimoto et al. (1997) Cancer Res., 57):2884-7). The hepsin transcript appears to be abundant in carcinoma tissue and is almost never expressed in normal adult tissue, including normal ovary. It has been suggested that hepsin is frequently overexpressed in ovarian tumors and therefore can be a candidate protease in the invasive process and growth capacity of ovarian tumor cells.

A serine protease-like gene, designated normal epithelial cell-specific 1 (NES1) (Liu et al. Cancer Res. 56:3371-3379 (1996)) has been identified. Although expression of the NES1 mRNA is observed in all normal and immortalized nontumorigenic epithelial cell lines, the majority of human breast cancer cell lines show a drastic reduction or a complete lack of its expression. The structural similarity of NES1 to polypeptides known to regulate growth factor activity and a negative correlation of NES1 expression with breast oncogenesis suggest a direct or indirect role for this protease-like gene product in the suppression of tumorigenesis.

Hence transmembrane and other serine proteases and other proteases appear to be involved in the etiology and pathogenesis of tumors. There is a need to further elucidate their role in these processes and to identify additional transmembrane proteases. Therefore, among the objects herein, it is an object herein to provide serine protease proteins and nucleic acids encoding such proteases that are involved in the regulation of or participate in tumorigenesis and/or carcinogenesis. It also is an object herein to provide prognostic, diagnostic, therapeutic screening methods using the such proteases and the nucleic acids encoding such proteases.

### SUMMARY OF THE INVENTION

Provided herein are proteins designated CVSP16 and protease domains thereof. CVSP16 is a member of the serine protease family and is expressed or active in breast, colon, lung, prostate, kidney, stomach, spleen, thyroid gland, trachea and pituitary gland and in tumor tissues and cancers, including colon, breast and prostate cancers, and in leukemias and lymphomas. Hence, as a protease it can be involved in tumor progression. By virtue of its functional activity it can be a therapeutic or diagnostic target. The expression and/or activation (or reduction in level of expression or activation) of the expressed protein (zymogen) of the this protein can be used to monitor cancer and cancer therapy.

The serine protease family includes members that are activated and/or expressed in tumor cells at different levels from non-tumor cells; and those from cells in which substrates therefor differ in tumor cells from non-tumor cells or otherwise alter the specificity of the serine protease (SP). The serine protease provided herein, designated herein as CVSP16, is a secreted protease. Protease domains and full-length protein, including the zymogen or inactive forms and activated forms, and uses thereof are also provided. Proteins encoded by splice variants are also provided. Nucleic acid molecules encoding the proteins and protease domains are also provided.

CVSP16 polypeptide is expressed as a secreted protein and may bind to cell surface receptors to function as a cell-surface bound protease, such as by binding thereto or by dimerization or multimerization with a membrane-bound or receptor-bound protein. CVSP16 polypeptides are serine proteases and exhibit catalytic activity and also can exhibit substrate and ligand binding activity. The CVSP16 proteases provided herein do not include a contiguous sequence of at 5 least amino acids from SEQ ID No. 21 inserted between residues corresponding to Q₆₆₀ and M₆₆₁ and/or the CVSP16 proteases contains at least two protease domains of a serine protease 16 (CVSP16) and includes at least 5 contiguous amino acids corresponding to residues 508-544 of SEQ ID No. 6. or contains the contiguous sequence Asn Asp Ser or Trp Asn Asp or Ser Cys Trp Asn Asp Ser or Cys Trp Asn Asp Ser or Gin Thr His or Leu Gln Thr His in the second protease domain.

An exemplified CVSP16 (see, e.g., SEQ ID Nos. 5 and 6, which set forth nucleic acid and amino acid sequences thereof), includes two protease domains, designated PD1 and PD2, respectively (see, e.g., PD1 amino acids 46 to 286 (which includes the R that is cleaved upon activation cleavage), and PD2 including amino acids 323 or 324 or 325 or 326 to 550 of SEQ ID No. 6). The exemplified CVSP16 contains a signal peptide sequence (e.g., aa 1 to aa 23 of SEQ ID No. 6) and a first trypsin-like serine protease domain designated herein as CVSP16 PD1 characterized by the presence of a protease activation cleavage site ( . . . R₄₆↓I₄₇VGGSNAQP . . . , where ↓ indicates protease activation cleavage site) at the beginning of the domain and the catalytic triad residues (H₈₇, D₁₃₉ and S₂₄₃) in 3 highly-conserved regions of the catalytic domain.

In addition CVSP16 has a second protease domain PD2, in which the catalytic histidine is replaced by a serine, indicating that the second protease domain has lower catalytic activity. The isolated protease domains as single chains are provided as are polypeptides that include such protease domains. In particular, a polypeptide that contains PD1 as the only CVSP16 portion is provided. Also provided polyepeptides that include PD1 and/or PD2, but do not include at least 5 contiguous amino acids from SEQ ID No. 21 and polypeptides that include PD1 or PD2, particularly polypeptides that include PD1 and/or PD2 as the only CVSP16 portion. Also included are CVSP16 proteases that contain at least two protease domains of a serine protease 16 (CVSP16) and include at least 5 contiguous amino acids corresponding to residues 508-544 of SEQ ID No. 6. or contains the contiguous sequence Asn Asp Ser or Trp Asn Asp or Ser Cys Trp Asn Asp Ser or Cys Trp Asn Asp Ser or Gin Thr His or Leu Gln Thr His in the second protease domain.

Isolated PD1 and/or PD2 are provided as single-chain molecules are as activated one, two or three chain molecules. Each of PD1 or PD2 can exhibit functional activity (catalytic, substrate binding and/or ligand binding activity) without undergoing activation cleavage and/or as single chains.

Nucleic acid encoding the CVSP16 protease and upstream nucleic acid is set forth in SEQ ID No. 5; and the encoded protein is set forth in SEQ ID No. 6.

The protease domains for use in the methods and assays provided herein do not have to result from activation, which produces a two or multi-chain activated product, but can be a single-chain polypeptide. Such polypeptides, although not the result of activation and not two-chain forms, can exhibit proteolytic (catalytic) activity. These protease domain polypeptides are used in assays to screen for agents that modulate an activity of the CVSP16.

Such assays are also provided herein. In exemplary assays, the effects of test compounds on the ability of the full length-single chain, multiple chain activated forms, or a protease domain, which is a single chain or a double chain activated form, of CVSP16 to proteolytically cleave a known substrate, typically a fluorescently, chromogenically or otherwise detectably labeled substrate, are assessed. Agents, generally compounds, particularly small molecules, that modulate an activity of the polypeptide (full length or protease domain(s) either single or double chain or multi-chain forms thereof) are candidate compounds for modulating an activity of a CVSP16. The protease domains and full length proteins also can be used to produce that bind to CVSP16s provided herein as well as single-chain protease-specific antibodies and/or multi-chain specific antibodies.

The protease domains provided herein include, but are not limited to, a single chain region having an N-terminus at a cleavage site for activation of a zymogen form, through the C-terminus, or C-terminal truncated portions thereof that exhibit proteolytic activity as a single-chain polypeptide in in vitro proteolysis assays of any CVSP16 provided herein, from a mammal, including a human, that, for example, is expressed in tumor cells at different levels from non-tumor cells. Such protease domains include single-chains PD1 and PD2 each having an N-terminus that corresponds to that resulting from activation cleavage (i.e. corresponding to amino acids 47 and 324, respectively of SEQ ID No. 6). Also provided are truncated versions thereof, particularly, C-terminal truncated versions, that exhibit proteolytic and/or substrate binding and/or ligand binding activity.

Also provided are muteins of a single-chain protease domain of CVSP16 particularly muteins in which a Cys residue (corresponding to residue no. 159) in a protease domain that is unpaired (i.e., free; does not form disulfide linkages with any other Cys residue within the same protease domain) is substituted with another amino acid substitution, generally with a conservative amino acid substitution or a substitution that does not eliminate an activity, and muteins in which a glycosylation site(s) is eliminated. Similarly, an unpaired cysteine (corresponding to residue no. 430 in SEQ ID No. 6, a residue that does not form disulfide linkages with any other Cys residue within the second protease domain) similarly can be substituted. Muteins in which other conservative amino acid substitutions in which catalytic activity or other functional activity is retained are also contemplated (see, e.g., Table 1, for exemplary amino acid substitutions).

Hence, provided herein are members of the family of serine proteases designated CVSP16, and functional domains, especially protease (or catalytic) domains thereof, muteins and other derivatives and analogs thereof. Also provided herein are nucleic acids encoding the CVSP16.

As noted, the nucleic acid and amino acid sequences of an exemplary CVSP16 are set forth in SEQ ID Nos. 5 and 6. Molecules with single or a plurality of amino acids insertions, deletions or substitutions are provides. Nucleic acid molecules that encode a single-chain protease domain or catalytically active portion thereof and also those that encode the full-length CVSP16 are provided. Also provided are nucleic acid molecules that hybridize to such CVSP16 encoding nucleic acid along at least about 70%, 80%, 90%, 95% or more of their full length and encode a protease domain or portion thereof. Hybridization is typically performed under conditions of at least low, generally at least moderate, and often high stringency; generally the hybridizing nucleic acid hybridizes along at least about 70%, 80%, 90%, 95% of its full length at the recited stringency.

Additionally provided herein are antibodies that specifically bind to the CVSP16, particularly the CVSP16s provided, including CVSP16s provided herein that does not include at least 5, 7, 10, 15, 20 or more contiguous amino acids from SEQ ID No. 21. Also provided are antibodies that specifically bind to CVSP16 proteases that contain at least two protease domains of a serine protease 16 (CVSP16) and include at least 5 contiguous amino acids corresponding to residues 508-544 of SEQ ID No. 6. or contains the contiguous sequence Asn Asp Ser or Trp Asn Asp or Ser Cys Trp Asn Asp Ser or Cys Trp Asn Asp Ser or Gln Thr His or Leu Gin Thr His in the second protease domain.

Included are antibodies that specifically bind to the protein or protease domain, including to the single and/or double chain forms thereof. Among the antibodies are two-chain-specific antibodies, and single-specific antibodies and neutralizing antibodies that inhibit functional activity (i.e., catalytic activity and/or substrate or binding activity). Also provided are antibodies that bind with at least 2-fold, 5-fold, 10-fold or 100-fold greater affinity to CVSP16 polypeptides that do not include a contiguous portion (5, 7, 10, 15, 20 or more amino acid residues) of the sequence of amino acids set forth in SEQ ID No. 21 compared to a CVSP polypeptide that includes SEQ ID No. 21. Typically the CVSP16 polypeptides that do not include the sequence of amino acids set forth in SEQ ID No. 21 do not include it between residues corresponding to Q₆₆₀ and M₆₆₁ (see, SEQ ID No. 6) and/or the CVSP16s polypeptides contain at least two protease domains of a serine protease 16 (CVSP16) and include at least 5 contiguous amino acids corresponding to residues 508-544 of SEQ ID No. 6. or contains the contiguous sequence Asn Asp Ser or Trp Asn Asp or Ser Cys Trp Asn Asp Ser or Cys Trp Asn Asp Ser or Gin Thr His or Leu Gin Thr His in the second protease domain.

CVSP16 polypeptides, including, but not limited to, splice variants thereof, domains, derivatives and analogs thereof are provided herein. Single-chain protease domains, where the N-terminal is that which would be generated by activation of the zymogen form are provided. Multi-chain, generally two- or three-chain activated forms of CVSP16 also are provided.

Also provided are cells, combinations, kits and articles of manufacture that contain the nucleic acid encoding the CVSP16 and/or the CVSP16. Further provided herein are prognostic, diagnostic, therapeutic screening methods using CVSP16 and the nucleic acids encoding CVSP16. Also provided are transgenic non-human animals bearing inactivated genes encoding CVSP16 and bearing the genes (or inserted cDNA) encoding the CVSP16, particularly under a non-native promoter control or on an exogenous element, such as a plasmid or artificial chromosome, are additionally provided herein. Also provided are nucleic acid molecules encoding each of CVSP16 and domains thereof.

Also provided are plasmids containing any of the nucleic acid molecules provided herein. Cells containing the plasmids are also provided. Such cells include, but are not limited to, bacterial cells, yeast cells, fungal cells, plant cells, insect cells and animal cells.

Also provided is a method of producing CVSP16 by growing the above-described cells under conditions whereby the CVSP16 is expressed by the cells, and recovering the expressed CVSP16 polypeptide. Methods for isolating nucleic acid encoding other CVSP16s are also provided.

Also provided are cells, generally eukaryotic cells, such as mammalian cells and yeast cells, in which the CVSP16 polypeptide is expressed by the cells, particularly under conditions in which it is secreted. Also provided are cells to which the CVSP16 polypeptide or a protease domain thereof is bound. Such cells are used in drug screening assays to identify compounds that modulate an activity of the CVSP16 polypeptide. These assays include in vitro binding assays, and transcription based assays in which signal transduction mediated by the bound CVSP16 is assessed.

The CVSP16 polypeptides (including those that include all or a portion of SEQ ID No. 21) are of interest for a variety of reasons. For example, they appear to be expressed and/or activated at different levels in tumor cells from normal cells, or to have functional activity that is different in tumor cells from normal cells, such as by an alteration in a substrate therefor, or a cofactor. The CVSP16 polypeptides (including those that include all or a portion of SEQ ID No. 21) are of interest for a variety of reasons. For example, they appear to be expressed and/or activated at different levels in tumor cells from normal cells, or to have functional activity that is different in tumor cells from normal cells, such as by an alteration in a substrate therefor, or a cofactor.

It is shown herein, that CVSP16 is expressed in cervical cancer. It also may be expressed in colon, breast, stomach, uterine, ovarian, lung and prostate tumors and in other tumors as well as in certain normal cells and tissues (see e.g., EXAMPLES for an exemplary tissue-specific expression profile). The expression and/or activation thereof and/or its presence above a predetermined among is a body fluid can be diagnostic or prognostic of cancer.

Hence the CVSP16 polypeptides provided herein can serve as diagnostic markers for certain tumors, particularly cervical cancers. The level of activated CVSP16 can be diagnostic of uterine, pancreatic, breast, lung, stomach, prostate or colon cancer or leukemia or other cancer.

Also provided herein are methods of modulating an activity of a CVSP16 and screening for compounds that modulate, including inhibit, antagonize, agonize or otherwise alter an activity of the CVSP16. Of particular interest is the proteolytic (catalytic) portion of the protein.

Thus, provided herein are prognostic, diagnostic and therapeutic screening methods using the CVSP16 and the nucleic acids encoding CVSP16. In particular, the prognostic, diagnostic and therapeutic screening methods are used for preventing, treating, or for finding agents useful in preventing or treating, tumors or cancers such as uterine, stomach, lung carcinoma, colon carcinoma and other cancers.

Methods of diagnosing a disease or disorder characterized by detecting an aberrant level of a CVSP16 in a subject is provided. The method can be practiced by measuring the level of the DNA, RNA, protein or functional activity of the CVSP16. An increase or decrease in the level of the DNA, RNA, protein or functional activity of the CVSP16, relative to the level of the DNA, RNA, protein or functional activity or any other suitable control, found in an analogous sample not having the disease or disorder (or other suitable control) is indicative of the presence of the disease or disorder in the subject.

Also provided are methods for screening for compounds that modulate an activity of CVSP16. The compounds are identified by contacting them with the CVSP16 or protease domain thereof and a substrate for the CVSP16. A change in the amount of substrate cleaved in the presence of the compounds and compared to that in the absence of the compound indicates that the compound modulates an activity of a CVSP16. Such compounds are selected for further analyses or for use to modulate the activity of the CVSP16, such as inhibitors or agonists. The compounds also can be identified by contacting the substrates with a cell that expresses the CVSP16 or with the CVSP16 or a proteolytically active portion thereof.

Computer based screening methods are also provided. In these methods, interactions between simulated test compounds computer simulated CVSP16 polypeptides are assessed, such as by computational docking or binding studies. Test compounds predicted to bind or otherwise interact with a CVSP16 polypeptide are selected as drug candidates. Further characterization and study, such as in vitro assays, can be performed.

Also provided herein are modulators of the activity of CVSP16, especially the modulators (i.e., antagonists, agonists, inhibitors), including antibodies and RNA molecules, and molecules obtained according to the screening methods provide herein. Such modulators can have use in treating cancerous conditions, and other neoplastic conditions.

Pharmaceutical compositions containing a protease domain and/or full-length or two protease domains or other domain of a CVSP16 polypeptide are provided herein in a pharmaceutically acceptable carrier or excipient are provided herein.

Also provided are articles of manufacture that contain CVSP16 polypeptide and/or a protease domain or protease domains of a CVSP16 as single-chain zymogen and activated forms and as full-length form zymogen or activated forms and other forms thereof. The articles contain a) packaging material; b) a CVSP16 polypeptide (or encoding nucleic acid), particularly a polypeptide containing a single chain protease domain thereof; and c) a label indicating that the article is for use in assays for identifying modulators of the activities of a CVSP16 polypeptide or for use in diagnostic assays.

Conjugates containing a) a CVSP16 polypeptide or protease domain in single chain from; and b) a targeting agent linked to the SP directly or via a linker, wherein the agent facilitates: i) affinity isolation or purification of the conjugate; ii) attachment of the conjugate to a surface; iii) detection of the conjugate; or iv) targeted delivery to a selected tissue or cell, is provided herein. The conjugate can contain a plurality of agents linked thereto. The conjugate can be a chemical conjugate; and it can be a fusion protein. Targeting agents include proteins and peptide fragments. The protein or peptide fragment can include a protein binding sequence, a nucleic acid binding sequence, a lipid binding sequence, a polysaccharide binding sequence, or a metal binding sequence.

Combinations are provided herein. The combination can include: a) an inhibitor of an activity of a CVSP16; and b) an anti-cancer treatment or agent. The SP inhibitor and the anti-cancer agent can be formulated in a single pharmaceutical composition or each is formulated in a separate pharmaceutical composition. The CVSP16 inhibitor can be an antibody or a fragment or binding portion thereof made against the CVSP16, such as an antibody that specifically binds to a protease domain, an inhibitor of CVSP16 production, or an inhibitor of CVSP16 membrane-localization or an inhibitor of CVSP16 activation. Other CVSP16 inhibitors include, but are not limited to, an antisense nucleic acid encoding the CVSP16, particularly a portion of a protease domain, a nucleic acid encoding at least a portion of a gene encoding the CVSP16 with a heterologous nucleotide sequence inserted therein such that the heterologous sequence inactivates the biological activity of the encoded CVSP16 or the gene encoding it. The portion of the gene encoding the CVSP16 typically will flank the heterologous sequence to promote homologous recombination with a genomic gene encoding the CVSP16.

Also, provided are methods for treating or preventing a tumor or cancer in a mammal by administering to a mammal an effective amount of an inhibitor of a CVSP16, whereby the tumor or cancer is treated or prevented. The CVSP16 inhibitor used in the treatment or for prophylaxis is administered with a pharmaceutically acceptable carrier or excipient. The mammal treated can be a human. The treatment or prevention method can additionally include administering an anti-cancer treatment or agent simultaneously with or subsequently or before administration of the CVSP16 inhibitor.

Also provided is a recombinant non-human animal in which an endogenous gene of a CVSP16 has been deleted or inactivated by homologous recombination or insertional mutagenesis of the animal or an ancestor thereof. A recombinant non-human animal is provided herein, where the gene of a CVSP16 is under control of a promoter that is not the native promoter of the gene or that is not the native promoter of the gene in the non-human animal or where the nucleic acid encoding the CVSP16 is heterologous to the non-human animal and the promoter is the native or a non-native promoter or the CVSP16 is on an extrachromosomal element, such as a plasmid or artificial chromosome.

Also provided are methods of treatments of tumors by administering a prodrug that is activated by CVSP16 that is expressed or active in tumor cells, particularly those in which its functional activity in tumor cells is greater than in non-tumor cells. The prodrug is administered and, upon administration, active CVSP16 expressed on cells cleaves the prodrug and releases active drug in the vicinity of these cells. The active anti-cancer drug accumulates in the vicinity of the tumor. This is particularly useful in instances in which CVSP16 is expressed or active in greater quantity, higher level or predominantly in tumor cells compared to other cells.

Also provided are methods of identifying a compound that binds to a form of CVSP16, by contacting a test compound with two or more forms; determining to which form or forms the compound binds; and if it binds to a form of CVSP16, further determining whether the compound has at least one of the following properties:
(i) inhibits activation of a single-chain zymogen form of CVSP16;
(ii) inhibits activity of a form; and/or
(iii) inhibits dimerization of the protein.
   The forms can be full length forms zymogen or activated forms, or a single or multi-chain protease domain or two protease domains or other form resulting from cleavage at an activation site.

Also provided are methods of diagnosing the presence of a premalignant lesion, a malignancy, or other pathologic condition in a subject, by obtaining a biological sample from the subject; exposing it to a detectable agent that binds to a multi-chain (i.e., two- or three-chain) or single-chain form of CVSP16, where the pathological condition is characterized by the presence or absence or amount of a three-chain, two-chain or single-chain form.

Methods of inhibiting tumor invasion or metastasis or treating a malignant or pre-malignant condition by administering an agent that inhibits activation of a zymogen form of CVSP16 or an activity of an activated form are provided. The conditions include, but are not limited to, a condition, such as a tumor of the uterus, stomach and also the breast, cervix, prostate, esophagus, lung, ovary and colon.

Methods for monitoring tumor progression and/or therapeutic effectiveness are also provided. The levels of activation or expression of CVSP16 or a protease domain or domains thereof are assessed, and the change in the level, reflects tumor progression and/or the effectiveness of therapy. Generally, as the tumor progresses the amount of CVSP16 in a body tissue or fluid sample increases; effective therapy reduces the level.

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

As used herein, protease refers to an enzyme catalyzing hydrolysis of proteins or peptides. It includes zymogen forms and activated single-, two- and three-chain forms thereof. For clarity reference to protease refers to all forms, and particular forms will be specifically designated.

As used herein, serine protease refers to a diverse family of proteases wherein a serine residue is involved in the hydrolysis of proteins or peptides. The serine residue can be part of the catalytic triad mechanism, which includes a serine, a histidine and an aspartic acid in the catalysis, or be part of the hydroxyl/E-amine or hydroxyl/α-amine catalytic dyad mechanism, which involves a serine and a lysine in the catalysis. Of particular interest are SPs of mammalian, including human, origin. Those of skill in this art recognize that, in general, single amino acid substitutions in non-essential regions of a polypeptide do not substantially alter biological activity (see, e.g., Watson et al. (1987) Molecular Biology of the Gene, 4th Edition, The Benjamin/Cummings Pub. co., p. 224).

As used herein, “transmembrane serine protease (MTSP)” refers to a family of transmembrane serine proteases that share common structural features as described herein (see, also Hooper et al. (2001) J. Biol. Chem. 276:857-860). Thus, reference, for example, to “MTSP” encompasses all proteins encoded by the MTSP gene family, including but are not limited to: MTSP3, MTSP4, MTSP6, MTSP7, MTSP9, MTSP10, MTSP20 or an equivalent molecule obtained from any other source or that has been prepared synthetically or that exhibits the same activity. Other MTSPs include, but are not limited to, corin, enteropeptidase, human airway trypsin-like protease (HAT), MTSP1, TMPRSS2 and TMPRSS4. Sequences of encoding nucleic acid molecules and the encoded amino acid sequences of exemplary MTSPs and/or domains thereof are set forth, for example in U.S. application Ser. No. 09/776,191 (SEQ ID Nos. 1-12, 49, 50 and 61-72 therein, published as International PCT application No. WO 01/57194; see also published International PCT application Nos. WO 02/072786 and WO 02/977267, and International PCT application Nos. PCT/US02/21208 and PCT/US02/15332). The term also encompass MTSPs with amino acid substitutions that do not substantially alter activity of each member and also encompasses splice variants thereof. Suitable substitutions, including, although not necessarily, conservative substitutions of amino acids, are known to those of skill in this art and can be made without eliminating a biological activity, such as the catalytic activity, of the resulting molecule.

As used herein, Type I MTSP refers to transmembrane proteins made with an N-terminal signal peptide that is cleaved so that the new N-terminus is on the extracytoplasmic side of the membrane. The original N-terminus likely stays on the cytoplasmic side, and cleavage occurs on the other side of the membrane. These proteins are anchored through a C-terminal membrane-spanning segment.

As used herein, Type II MTSP refers to transmembrane proteins that are synthesized with N-terminal or internal signal peptides that are not cleaved and that serve as a membrane anchor.

As used herein, a “protease domain of a CVSP”, particularly CVSP16, refers to a domain of a SP that exhibits proteolytic activity and shares homology and structural features with the chymotrypsin/trypsin family protease domains. Hence it is at least the minimal portion of the domain that exhibits proteolytic activity (or other functional activity, such as ligand or substrate binding) as assessed by standard in vitro assays. Those of skill in this art recognize that a protease domain is the portion of the protease that is structurally equivalent to the trypsin or chymotrypsin fold. Contemplated herein are polypeptides that include such protease domains and catalytically active portions thereof. Also provided are truncated forms of a protease domain that include the smallest fragment thereof that acts catalytically as a single-chain form.

Thus, as used herein a protease domain of a CVSP16, whenever referenced herein, includes at least one or all of or any combination of or a catalytically active portion of a CVSP16 polypeptide as defined herein. A protease domain of a CVSP16 protein refers to a protease domain of a CVSP16 that exhibits serine proteolytic activity. Hence it is at least a minimal portion of the protein that exhibits proteolytic activity (or binds to a substrate or ligand) as a single-chain form or as an activated form. as assessed by standard assays in vitro. It refers, herein, to a single chain form and also multi-chain activated forms. Exemplary protease domains include at least a sufficient portion of a sequence of amino acids set forth in SEQ ID No. 6 (encoded by nucleotides in SEQ ID No. 5) to exhibit protease activity.

Protease domains of CVSPs vary in size and constitution, including insertions and deletions in surface loops. They retain conserved structure, including at least one of the active site triad, primary specificity pocket, oxyanion hole and/or other features of serine protease domains of proteases. For purposes herein, a protease domain is a single chain portion of a CVSP16, as defined herein, but is homologous in its structural features and retention of sequence of similarity or homology to the protease domain of chymotrypsin or trypsin. Smaller portions of the protease domains, particularly the single chain domains, thereof that retain protease activity are contemplated. Such smaller versions will generally be C-terminal truncated versions of the protease domains. Multi-chain, typically two-chain forms, resulting from activation cleavage are also contemplated. The polypeptide can exhibit proteolytic activity as a single chain.

Thus, for purposes herein, a protease domain is a portion of a CVSP16, as defined herein, and shares common features and homologies with protease domains of other SPs. As with the larger class of enzymes of the chymotrypsin (S1) fold (see, e.g., Internet accessible MEROPS database), the CVSP16 protease domains share a high degree of amino acid sequence identity. The His, Asp and Ser residues necessary for activity are present in conserved motifs in PD1 and in modified form in PD2. An activation site, whose cleavage creates the N-terminus of the protease domain in two-chain forms has a conserved motif and location relative to other sites in the domain and can be identified. Domains that include some of these conserved features, such as the His, but that exhibit any proteolytic activity are considered protease domains herein. An activation site, which results in the N-terminus of a second chain in the multi-chain forms has a conserved motif and/or location within the polypeptide.

Exemplary of a protease domain of a CVSP16 is a polypeptide set forth as amino acids 46 to 286 or including amino acids 326 to 550, particularly 323-550, of SEQ ID No. 6, which can provided as a single-chain isolated molecule or as molecule that contains both domains but not the full-length CVSP16. The single-chain form can be a zymogen or activated form in which the N-terminus corresponds to the N-terminus produced by activation cleavage.

As used herein, the catalytically active domain of CVSP16 refers to the protease domain. Reference to a protease domain of a CVSP16 includes the single, two- and multi-chain forms of any of these proteins. A zymogen form of each protein is an inactive single chain form, which can be converted to an active multi-chain form by cleavage. A protease domain also can be converted to a multi-chain form.

Significantly, at least in vitro, the single chain forms of the SPs and catalytic domains or proteolytically active portions thereof (typically C-terminal truncations) thereof exhibit protease activity. Hence provided herein are isolated single chain forms of protease domains of SPs and their use in in vitro drug screening assays for identification of agents that modulate an activity thereof.

For the protease domains, residues at the N-terminus can be critical for activity, since it has been shown that the free amino group at the N-terminus of such proteases is essential for formation of the catalytically active conformation upon activation cleavage of the zymogen form of the protease. A protease domain of the single chain form of the CVSP16 protease can be catalytically active. Hence a protease domain generally requires N-terminal amino acids; the C-terminal portion can be truncated. The amount that can be removed can be determined empirically by testing the protein for protease activity in an in vitro assay that assesses catalytic cleavage.

Thus, as used herein, the catalytically active domain of a CVSP refers to a protease domain. Typically this refers to a single-chain form of a protease domain. Multi-chain, particularly two chain forms of a protease domain also are contemplated; such domains are necessarily longer since they include one or more amino acids on the N-terminus side of the activation cleavage site as a second chain. A zymogen form of each protease domain and the protease is a single chain, which is converted to an activated, typically a multi-chain form, such as a two or a three chain form or an active two or three-chain form by activation cleavage of one or both protease domains. Single-chain forms with the N-terminus that corresponds to the activation cleavage site can be active in vitro and are provided.

As used herein, by active form is meant a form active in vivo and/or in vitro. Single chain forms of the SPs and the catalytic domains or proteolytically active portions thereof (typically C-terminal truncations) exhibit protease activity. For example, a polypeptide containing the protease domain can exist as an activated three-chain, two-chain or a single chain active form. The active single chain, two chain and three-chain forms of a CVSP16 and catalytic domains or proteolytically active portions thereof can exhibit protease activity. Among the polypeptides provided herein, are isolated single-chain forms, two-chain and three-chain forms of CVSP16 polypeptides that include protease domains and their use, for example, in in vitro drug screening assays for identification of agents that modulate the activity thereof.

There are two types of single-chain forms; active and zymogen forms. When cleaved at the activation site, there only one single chain active forms. Any form that results from activation cleavage or that has an N-terminus at a site that is the site of cleavage is an active single-chain forms. The N-terminus of an activated form must result from activation cleavage and be at that site. Any other single-chain form, which has a A different N-terminus is a zymogen.

As used herein, a CVSP16 whenever referenced herein, includes at least one or all of or any combination of:
a) a polypeptide encoded by the sequence of nucleotides set forth in SEQ ID No. 5;
b) a polypeptide encoded by a sequence of nucleotides that hybridizes under conditions of low, moderate or high stringency to the sequence of nucleotides set forth in SEQ ID No. 5, wherein the encoded polypeptide does not include at least 5 or at least 6 or at least 7 or at least 8 contiguous or at least 9 or at least 10 contiguous amino acids from SEQ ID No. 21 up to all of SEQ ID No. 21, and particularly do not include any amino acids therefrom between amino acids corresponding to Gln₆₆₀ and Met₆₆₁ of SEQ ID No. 6;
c) a polypeptide that contains the sequence of amino acids set forth in SEQ ID No. 6;
d) a polyeptide that contains at least two protease domains of a serine protease 16 (CVSP16) and includes at least 5 contiguous amino acids corresponding to residues 508-544 of SEQ ID No. 6. or contains the contiguous sequence Asn Asp Ser or Trp Asn Asp or Ser Cys Trp Asn Asp Ser or Cys Trp Asn Asp Ser or Gin Thr His or Leu Gin Thr His in the second protease domain;
e) a polypeptide that contains a sequence of amino acids having at least about 60%, 70%, 80%, 90% or about 95% sequence identity with the sequence of amino acids set forth in SEQ ID No. 6, wherein the polypeptide does not include at least 5 or at least 6 or at least 7 or at least 8 contiguous or at least 9 or at least 10 contiguous amino acids from SEQ ID No. 21 up to all of SEQ ID No. 21, and particularly does not include any amino acids therefrom between amino acids corresponding to Gln₆₆₀ and Met₆₆₁ of SEQ ID No. 6;
f) the polypeptide is encoded by a sequence of nucleotides that hybridizes under conditions of at least moderate, and can be high, stringency along at least 70% of its full length to a sequence of nucleotides than encodes a polypeptide of any of a)-e), wherein the polypeptide does not include at least 5 or at least 6 or at least 7 or at least 8 contiguous or at least 9 or at least 10 contiguous amino acids from SEQ ID No. 21 up to all of SEQ ID No. 21, and particularly do not include any amino acids therefrom between amino acids corresponding to Gln₆₆₀ and Met₆₆₁ of SEQ ID No. 6;
g) the polypeptide has at least 60%, 70%, 80%, 90% or about 95% sequence identity with a polypeptide of any of a)-f), wherein the polypeptide does not include at least 5 or at least 6 or at least 7 or at least 8 contiguous or at least 9 or at least 10 contiguous amino acids from SEQ ID No. 21 up to all of SEQ ID No. 21, and particularly do not include any amino acids therefrom between amino acids corresponding to Gln₆₆₀ and Met₆₆₁ of SEQ ID No. 6; and/or
h) a polypeptide encoded by a splice variant of a sequence of nucleotides that encodes a CVSP16 polypeptide provided herein. Smaller portions thereof that retain protease activity are contemplated.

An exemplary CVSP16 polypeptide includes the sequence of amino acids set forth in SEQ ID No. 6; the protease domains are included in the sequence of amino acids set forth in SEQ ID No. 6. Smaller portions thereof that retain protease activity are contemplated.

Included among the CVSP16 polypeptides, as provided herein are those that include a protease domain of serine protease 16 (CVSP16) or a catalytically active portion thereof, where:
the polypeptide does not include at least 5 or at least 6 or at least 7 or at least 8 contiguous or at least 9 or at least 10 contiguous amino acids from SEQ ID No. 21, and particularly do not include any amino acids therefrom between amino acids corresponding to Gln₆₆₀ and Met₆₆₁ of SEQ ID No. 6;
the polypeptide contains one, two or three chains; and
a protease domain contains amino acids 46-286 or 326-550 of SEQ ID No. 6 or amino acids that share at least about 60%, 70%, 80%, 90% or 95% homology to amino acids 46-286 or 326-550 of SEQ ID No. 6. Exemplary of the protease domains are PD1 (aa 46 to aa 286 of SEQ ID No. 6); and PD2 (aa 323 to aa 550 SEQ ID No. 6). Any polypeptide provided herein that includes PD1 and PD2 of a CVSP16 does not include at least 5, 6, 10, 15 or 20 contiguous amino acids from SEQ ID No. 21, and particularly do not include any amino acids therefrom between amino acids (if present) corresponding to Gln₆₆₀ and Met₆₆₁ of SEQ ID No. 6.

The CVSP16 can be from any animal, particularly a mammal, and includes but are not limited to, primates including humans, gorillas and monkeys; rodents, such as mice and rats; fowl, such as chickens; ruminants, such as goats, cows, deer, sheep; ovine, such as pigs and other animals. The full length zymogen or a multi-chain activated form is contemplated as is any domain thereof, including a protease domain, which can be a three-chain activated form, two-chain activated form, or a single chain activated or zymogen form. An exemplary CVSP16 protein includes a sequence of amino acids set forth in SEQ ID No. 6 that includes a protease domain or a catalytically active portion thereof.

CVSP16s of interest include those that are activated and/or expressed in tumor cells at a level or amount different, typically higher, from those in non-tumor cells; and those from cells in which substrates therefor differ in tumor cells from non-tumor cells or differ with respect to the substrates, co-factors or receptors, or otherwise alter the activity or specificity of the MTSP.

By active form is meant a form that is functionally active in vivo and/or in vitro. Included among these are single-chain forms of a protease domain (i.e., those with an N-terminus corresponding to the normal activation cleavage site) that are active in vitro, including in assays for protease activity and/or substrate or ligand binding, and are used for screening assays, and also can be used as immunogens for preparing antibodies.

As used herein, a CVSP16 and/or a protease domain(s) thereof also can exist as a two-chain or other multi-chain (i.e., two or three chains) activated form. At least in vitro, unactivated and active single chain forms of the SPs and other unactivated forms and the catalytic domains or proteolytically or functionally active portions thereof (typically C-terminal truncations) exhibit protease or other functional activity (i.e., catalytic activity and/or other activity, such as substrate or ligand binding). Hence provided herein are isolated single chain forms of protease domains of SPs, and also multi-chain forms, and their use in in vitro drug screening assays for identification of agents that modulate the activity thereof.

As used herein, a functionally active CVSP16 polypeptide or portion thereof exhibits at least one of catalytic activity, substrate binding activity or ligand binding activity. As described, for example, PD1 and PD2 exhibit catalytic (protease) activity. The CVSP16 polypeptide can be active in vitro or in vivo.

Also contemplated are nucleic acid molecules that encode a polypeptide that has proteolytic activity in an in vitro proteolysis assay and that have at least 80%, 85%, 90% or 95% sequence identity with the full length of a protease domain of a CVSP16 polypeptide as provided herein, or that hybridize along at least about 70%, 80%, 90% and 95% of their full length to a nucleic acids that encode a protease domain, particularly under conditions of moderate, generally high, stringency.

As used herein, a zymogen form of a full-length CVSP16 poly-peptide or a truncated CVSP16 polypeptide containing one or a plurality of protease domains is one in which at least one of the domains has not undergone activation cleavage. A zymogen form refers to a form in which at least one protease domain remains unactivated.

As used herein, activation cleavage refers to the cleavage of the protease at the N-terminus of the protease domain (generally between an R and I or an R and a V or elsewhere as for PD2 herein). By virtue of the Cys-Cys pairing between a Cys outside the protease domain and a Cys in the protease domain upon cleavage the resulting polypeptide has at least two chains. Cleavage can be effected by another protease or autocatalytically. In the exemplified CVSP16, the following cysteine pairings are noted: C₇₂-C₈₈, C₁₇₃-C₂₄₉, C₂₀₆-C₂₂₈, C₂₃₉-C₂₆₇, C₃₄₈-C₃₆₄, C₄₄₄-C₅₁₆, C₄₇₂-C₄₉₄ and C₅₀₆-C₅₃₄. In addition, an unpaired cysteine (C₁₅₉) in the first protease domain should pair with C₃₈ (by unpaired is meant unpaired with a Cys in the particular domain). An unpaired cysteine (C₄₃₀) in the second protease domain should pair with C₃₂₅ or C₃₁₈. As a result the protease, upon activation cleavage can contain multiple chains, including two or three or more chains, formed by virtue of pairing between the unpaired cysteine in a protease domain with a cysteine outside a protease domain.

As used herein, a two-chain form of a protease domain refers to a two-chain form that is formed from a single chain-form following activation cleavage or other cleavage of the protease. In such forms the Cys pairing between, in this instance, a Cys outside a protease domain and an upaired Cys in PD1 or PD2, links a protease domain to the remainder of the polypeptide and the activation cleavage cleaves the chain. A two-chain protease domain form refers to any form in which the “remainder of the polypeptide” is shortened or cleaved from the full-length and includes a Cys from outside a protease domain. The three chain form refers to form in which two of the protease domains are activated in this manner.

As used herein, a form of a CVSP16 is one or more of a single chain form, a two-chain form, a three chain form and/or other multi-chain form and the form is activated or is a zymogen or includes one or more activated domains.

As used herein, a human protein is one encoded by nucleic acid, such as DNA, present in the genome of a human, including all allelic variants and conservative variations as long as they are not variants found in other mammals.

As used herein, a “nucleic acid encoding a protease domain or catalytically active portion of a SP” refers to a nucleic acid encoding only the recited single chain protease domain or active portion thereof, and not the other contiguous portions of the SP as a continuous sequence.

As used herein, catalytic activity refers to the activity of the SP as a serine protease. Function of the SP refers to its function in tumor biology, including promotion of or involvement in initiation, growth or progression of tumors, and also roles in signal transduction. Catalytic activity refers to the activity of the SP as a protease as assessed in in vitro proteolytic assays that detect proteolysis of a selected substrate.

As used herein, a CUB domain is a motif homologous to domains that mediate protein-protein interactions in complement components C1r/C1s. CUB domains have been identified in other proteases and various proteins involved in developmental processes.

As used herein, a “propeptide” or “pro sequence” is sequence of amino acids positioned at the amino terminus of a mature biologically active polypeptide. When so-positioned, the resulting polypeptide is called a zymogen. Zymogens, generally, are inactive and can be converted to mature active polypeptides by catalytic or autocatalytic cleavage of the propeptide from the zymogen.

As used herein, a zymogen is an inactive precursor of a proteolytic enzyme. Such precursors are generally larger, although not necessarily larger than the active form. With reference to serine proteases, zymogens are converted to active enzymes by specific cleavage, including catalytic and autocatalytic cleavage, or by binding of an activating co-factor, which generates an active enzyme. A zymogen, thus, is an enzymatically inactive protein that is converted to a proteolytic enzyme by the action of an activator. Cleavage can be effected autocatalytically.

As used herein, “disease or disorder” refers to a pathological condition in an organism resulting from, e.g., infection or genetic defect, and characterized by identifiable symptoms.

As used herein, neoplasm (neoplasia) refers to abnormal new growth, and thus means the same as tumor, which can be benign or malignant. Unlike hyperplasia, neoplastic proliferation persists even in the absence of the original stimulus.

As used herein, neoplastic disease refers to any disorder involving cancer, including tumor development, growth, metastasis and progression.

As used herein, cancer is a general term for diseases caused by or characterized by any type of malignant tumor.

As used herein, malignant, as applies to tumors, refers to primary tumors that have the capacity of metastasis with loss of growth control and positional control.

As used herein, an anti-cancer agent (used interchangeably with “anti-tumor or anti-neoplastic agent”) refers to any agents used in anti-cancer treatment. These include any agents, when used alone or in combination with other compounds, that can alleviate, reduce, ameliorate, prevent, or place or maintain in a state of remission of clinical symptoms or diagnostic markers associated with neoplastic disease, tumors and cancer, and can be used in methods, combinations and compositions provided herein. Non-limiting examples of anti-neoplastic agents include anti-angiogenic agents, alkylating agents, antimetabolite, certain natural products, platinum coordination complexes, anthracenediones, substituted ureas, methylhydrazine derivatives, adrenocortical suppressants, certain hormones, antagonists and anti-cancer polysaccharides.

As used herein, a splice variant refers to a variant produced by differential processing of a primary transcript of genomic nucleic acid, such as DNA, that results in more than one type of mRNA. Polypeptides encoded by splice variants of the disclosed nucleic acids encoding CVSP16 polypeptides are provided herein.

As used herein, angiogenesis is intended to broadly encompass the totality of processes directly or indirectly involved in the establishment and maintenance of new vasculature (neovascularization), including, but not limited to, neovascularization associated with tumors.

As used herein, anti-angiogenic treatment or agent refers to any therapeutic regimen and compound, when used alone or in combination with other treatment or compounds, that can alleviate, reduce, ameliorate, prevent, or place or maintain in a state of remission of clinical symptoms or diagnostic markers associated with undesired and/or uncontrolled angiogenesis. Thus, for purposes herein an anti-angiogenic agent refers to an agent that inhibits the establishment or maintenance of vasculature. Such agents include, but are not limited to, anti-tumor agents, and agents for treatments of other disorders associated with undesirable angiogenesis, such as diabetic retinopathies, restenosis, hyperproliferative disorders and others.

As used herein, non-anti-angiogenic anti-tumor agents refer to anti-tumor agents that do not act primarily by inhibiting angiogenesis.

As used herein, pro-angiogenic agents are agents that promote the establishment or maintenance of the vasculature. Such agents include agents for treating cardiovascular disorders, including heart attacks and strokes.

As used herein, undesired and/or uncontrolled angiogenesis refers to pathological angiogenesis wherein the influence of angiogenesis stimulators outweighs the influence of angiogenesis inhibitors. As used herein, deficient angiogenesis refers to pathological angiogenesis associated with disorders where there is a defect in normal angiogenesis resulting in aberrant angiogenesis or an absence or substantial reduction in angiogenesis.

As used herein, by homologous means about greater than 25% nucleic acid sequence identity, such as 25%, 40%, 60%, 70%, 80%, 90% or 95%. If necessary the percentage homology will be specified. The terms “homology” and “identity” are often used interchangeably but homology for proteins can include conservative amino acid changes. In general, sequences (protein or nucleic acid) are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence identity, the number of identical amino acids is determined by standard alignment algorithm programs, and used with default gap penalties established by each supplier. Substantially homologous nucleic acid molecules would hybridize typically at moderate stringency or at high stringency all along the length of the nucleic acid or along at least about 70%, 80% or 90% of the full length nucleic acid molecule of interest. Also contemplated are nucleic acid molecules that contain degenerate codons in place of codons in the hybridizing nucleic acid molecule. (For proteins, for determination of homology conservative amino acids can be aligned as well as identical amino acids; in this case percentage of identity and percentage homology vary).

Whether any two nucleic acid molecules have nucleotide sequences that are at least 80%, 85%, 90%, 95%, 96%, 97%, 98% or 99% “identical” can be determined using known computer algorithms such as the “FAST A” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, a GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Bio-medical Research Foundation, pp. 353-358 (1979); (2) a penalty of 3.0 for each gap and an additional 0.10 penalty for each symbol in each gap; and (3) no penalty for end gaps. Therefore, as used herein, the term “identity” represents a comparison between a test and a reference polypeptide or polynucleotide.

As used herein, recitation that amino acids of a polypeptide correspond to amino acids in a disclosed sequence, such as amino acids Q660 and M661 of SEQ ID No. 6, refers to amino acids identified upon alignment of the polypeptide with the disclosed sequence to maximize identity or homology (where conserved amino acids are aligned) using a standard alignment algorithm, such as the GAP algorithm.

As used herein, the term “at least 90% identical to” refers to percent identities from 90 to 100% relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often without relying on software.

As used herein, primer refers to an oligonucleotide containing two or more deoxyribonucleotides or ribonucleotides, typically more than three, from which synthesis of a primer extension product can be initiated. Experimental conditions conducive to synthesis include the presence of nucleoside triphosphates and an agent for polymerization and extension, such as DNA polymerase, and a suitable buffer, temperature and pH.

As used herein, animal includes any animal, such as, but are not limited to primates including humans, gorillas and monkeys; rodents, such as mice and rats; fowl, such as chickens; ruminants, such as goats, cows, deer, sheep; ovine, such as pigs and other animals. Non-human animals exclude humans as the contemplated animal. The SPs provided herein are from any source, animal, plant, prokaryotic and fungal. Most CVSP16s are of animal origin, including mammalian origin.

As used herein, genetic therapy or gene therapy involves the transfer of heterologous nucleic acid, such as DNA, into certain cells, target cells, of a mammal, particularly a human, with a disorder or conditions for which such therapy is sought. The nucleic acid, such as DNA, is introduced into the selected target cells, such as directly or in a vector or other delivery vehicle, in a manner such that the heterologous nucleic acid, such as DNA, is expressed and a therapeutic product encoded thereby is produced. Alternatively, the heterologous nucleic acid, such as DNA, can in some manner mediate expression of DNA that encodes the therapeutic product, or it can encode a product, such as a peptide or RNA that in some manner mediates, directly or indirectly, expression of a therapeutic product. Genetic therapy also can be used to deliver nucleic acid encoding a gene product that replaces a defective gene or supplements a gene product produced by the mammal or the cell in which it is introduced. The introduced nucleic acid can encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous nucleic acid, such as DNA, encoding the therapeutic product can be modified prior to introduction into the cells of the afflicted host in order to enhance or otherwise alter the product or expression thereof. Genetic therapy also can involve delivery of an inhibitor or repressor or other modulator of gene expression.

As used herein, heterologous nucleic acid is nucleic acid that is not normally produced in vivo by the cell in which it is expressed or that is produced by the cell but is at a different locus or expressed differently or that mediates or encodes mediators that alter expression of endogenous nucleic acid, such as DNA, by affecting transcription, translation, or other regulatable biochemical processes. Heterologous nucleic acid is generally not endogenous to the cell into which it is introduced, but has been obtained from another cell or prepared synthetically. Heterologous nucleic acid can be endogenous, but is nucleic acid that is expressed from a different locus or altered in its expression. Generally, although not necessarily, such nucleic acid encodes RNA and proteins that are not normally produced by the cell or in the same way in the cell in which it is expressed. Heterologous nucleic acid, such as DNA, also can be referred to as foreign nucleic acid, such as DNA. Thus, heterologous nucleic acid or foreign nucleic acid includes a nucleic acid molecule not present in the exact orientation or position as the counterpart nucleic acid molecule, such as DNA, is found in a genome. It also can refer to a nucleic acid molecule from another organism or species (i.e., exogenous).

Any nucleic acid, such as DNA, that one of skill in the art would recognize or consider as heterologous or foreign to the cell in which the nucleic acid is expressed is herein encompassed by heterologous nucleic acid; heterologous nucleic acid includes exogenously added nucleic acid that also is expressed endogenously. Examples of heterologous nucleic acid include, but are not limited to, nucleic acid that encodes traceable marker proteins, such as a protein that confers drug resistance, nucleic acid that encodes therapeutically effective substances, such as anti-cancer agents, enzymes and hormones, and nucleic acid, such as DNA, that encodes other types of proteins, such as antibodies. Antibodies that are encoded by heterologous nucleic acid can be secreted or expressed on the surface of the cell in which the heterologous nucleic acid has been introduced.

As used herein, a therapeutically effective product for gene therapy is a product that is encoded by heterologous nucleic acid, typically DNA, that, upon introduction of the nucleic acid into a host, a product is expressed that ameliorates or eliminates the symptoms, manifestations of an inherited or acquired disease or that cures the disease. Also included are biologically active nucleic acid molecules, such as RNAi and antisense.

As used herein, recitation that a polypeptide consists essentially of the protease domain means that the only SP portion of the polypeptide is a protease domain or a catalytically active portion thereof. The polypeptide can optionally, and generally will, include additional non-SP-derived sequences of amino acids.

As used herein, cancer or tumor treatment or agent refers to any therapeutic regimen and/or compound that, when used alone or in combination with other treatments or compounds, can alleviate, reduce, ameliorate, prevent, or place or maintain in a state of remission of clinical symptoms or diagnostic markers associated with deficient angiogenesis.

As used herein, domain refers to a portion of a molecule, e.g., proteins or the encoding nucleic acids, that is structurally and/or functionally distinct from other portions of the molecule.

As used herein, nucleic acids include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, a probe or primer based on a nucleotide sequence disclosed herein, includes at least 10, 14, typically at least 16 contiguous nucleotides of SEQ ID No. 5, and probes of at least 30, 50 or 100 contiguous nucleotides of SEQ ID No. 5. The length of the probe or primer required for unique hybridization is a function of the complexity of the genome of interest.

As used herein, nucleic acid encoding a fragment or portion of a SP refers to a nucleic acid encoding only the recited fragment or portion of a SP, and not the other contiguous portions of the SP.

As used herein, operative linkage of heterologous nucleic to regulatory and effector sequences of nucleotides, such as promoters, enhancers, transcriptional and translational stop sites, and other signal sequences refers to the relationship between such nucleic acid, such as DNA, and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA. Thus, operatively linked or operationally associated refers to the functional relationship of nucleic acid, such as DNA, with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it can be necessary to remove, add or alter 5′ untranslated portions of the clones to eliminate extra, potentially inappropriate alternative translation initiation (i.e., start) codons or other sequences that can interfere with or reduce expression, either at the level of transcription or translation. Alternatively, consensus ribosome binding sites (see, e.g., Kozak J. Biol. Chem. 266:19867-19870 (1991)) can be inserted immediately 5′ of the start codon and can enhance expression. The desirability of (or need for) such modification can be empirically determined.

As used herein, a sequence complementary to at least a portion of an RNA, with reference to antisense oligonucleotides, means a sequence having sufficient complementarity to be able to hybridize with the RNA, generally under moderate or high stringency conditions, forming a stable duplex; in the case of double-stranded SP antisense nucleic acids, a single strand of the duplex DNA (or dsRNA) can thus be tested, or triplex formation can be assayed. The ability to hybridize depends on the degree of complementarily and the length of the antisense nucleic acid. Generally, the longer the hybridizing nucleic acid, the more base mismatches with a SP encoding RNA it can contain and still form a stable duplex (or triplex, as the case can be). One skilled in the art can ascertain a tolerable degree of mismatch by use of standard procedures to determine the melting point of the hybridized complex.

For purposes herein, amino acid substitutions, deletions and/or insertions, can be made in any of CVSPs and protease domains thereof provided that the resulting protein exhibits protease activity or other activity (or, if desired, such changes can be made to eliminate activity). Muteins can be made by making conservative amino acid substitutions and also non-conservative amino acid substitutions. For example, amino acid substitutions that desirably or advantageously alter properties of the proteins can be made. In one embodiment, mutations that prevent degradation of the polypeptide can be made. Many proteases cleave after basic residues, such as R and K; to eliminate such cleavage, the basic residue is replaced with a non-basic residue. Interaction of the protease with an inhibitor can be blocked while retaining catalytic activity by effecting a non-conservative change at the site of interaction of the inhibitor with the protease. Other activities also can be altered. For example, receptor binding can be altered without altering catalytic activity.

Amino acid substitutions contemplated include conservative substitutions, such as those set forth in Table 1, which do not eliminate proteolytic activity. As described herein, substitutions that alter properties of the proteins, such as removal of cleavage sites and other such sites are also contemplated; such substitutions are generally non-conservative, but can be readily effected by those of skill in the art.

Suitable conservative substitutions of amino acids are known to those of skill in this art and can be made generally without altering the biological activity, for example enzymatic activity, of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Also included within the definition, is the catalytically active fragment of a SP, particularly a single chain protease portion. Conservative amino acid substitutions are made, for example, in accordance with those set forth in TABLE 1 as follows:

TABLE 1

| Original residue | Conservative substitution |
|---|---|
| Ala (A) | Gly; Ser, Abu |
| Arg (R) | Lys, orn |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val; Met; Nle; Nva |
| Leu (L) | Ile; Val; Met; Nle; Nv |
| Lys (K) | Arg; Gln; Glu |
| Met (M) | Leu; Tyr; Ile; NLe Val |
| Ornithine | Lys; Arg |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu; Met; Nle; Nv |

Other substitutions are also permissible and can be determined empirically or in accord with known conservative substitutions.

As used herein, Abu is 2-aminobutyric acid; Orn is ornithine.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

As used herein, amelioration of the symptoms of a particular disorder by administration of a particular pharmaceutical composition refers to any lessening, whether permanent or temporary, lasting or transient that can be attributed to or associated with administration of the composition.

As used herein, antisense polynucleotides refer to synthetic sequences of nucleotide bases complementary to mRNA or the sense strand of double-stranded DNA. Admixture of sense and antisense polynucleotides under appropriate conditions leads to the binding of the two molecules, or hybridization. When these polynucleotides bind to (hybridize with) mRNA, inhibition of protein synthesis (translation) occurs. When these polynucleotides bind to double-stranded DNA, inhibition of RNA synthesis (transcription) occurs. The resulting inhibition of translation and/or transcription leads to an inhibition of the synthesis of the protein encoded by the sense strand. Antisense nucleic acid molecules typically contain a sufficient number of nucleotides to specifically bind to a target nucleic acid, generally at least 5 contiguous nucleotides, often at least 14 or 16 or 30 contiguous nucleotides or modified nucleotides complementary to the coding portion of a nucleic acid molecule that encodes a gene of interest, for example, nucleic acid encoding a single chain protease domain of a SP.

As used herein, an array refers to a collection of elements, such as antibodies, containing two, three or more members. An addressable array is one in which the members of the array are identifiable, typically by position on a solid phase support. Hence, in general the members of the array are immobilized on discrete identifiable loci on the surface of a solid phase.

As used herein, antibody refers to an immunoglobulin, whether natural or partially or wholly synthetically produced, including any derivative thereof that retains the specific binding ability of the antibody. Hence antibody includes any protein having a binding domain that is homologous or substantially homologous to an immunoglobulin binding domain. Antibodies include members of any immunoglobulin class, including IgG, IgM, IgA, IgD and IgE.

As used herein, antibody fragment refers to any derivative of an antibody that is less then full length, retaining at least a portion of the full-length antibody's specific binding ability. Examples of antibody fragments include, but are not limited to, Fab, Fab′, F(ab)₂, single-chain Fvs (scFV), FV, dsFV diabody and Fd fragments. The fragment can include multiple chains linked together, such as by disulfide bridges. An antibody fragment generally contains at least about 50 amino acids and typically at least 200 amino acids.

As used herein, a Fv antibody fragment is composed of one variable heavy chain domain (V_{H}) and one variable light chain domain linked by noncovalent interactions.

As used herein, a dsFV refers to an Fv with an engineered intermolecular disulfide bond, which stabilizes the V_{H}-V_{L} pair.

As used herein, a F(ab)₂ fragment is an antibody fragment that results from digestion of an immunoglobulin with pepsin at pH 4.0-4.5; it can be recombinantly produced to produce the equivalent fragment.

As used herein, Fab fragments are antibody fragments that result from digestion of an immunoglobulin with papain; it can be recombinantly produced to produce the equivalent fragment.

As used herein, scFVs refer to antibody fragments that contain a variable light chain (V_{L}) and variable heavy chain (V_{H}) covalently connected by a polypeptide linker in any order. The linker is of a length such that the two variable domains are bridged without substantial interference. Included linkers are (Gly-Ser)ₙ residues with some Glu or Lys residues dispersed throughout to increase solubility.

As used herein, humanized antibodies refer to antibodies that are modified to include human sequences of amino acids so that administration to a human does not provoke an immune response. Methods for preparation of such antibodies are known. For example, to produce such antibodies, the encoding nucleic acid in the hybridoma or other prokaryotic or eukaryotic cell, such as an E. coli or a CHO cell, that expresses the monoclonal antibody is altered by recombinant nucleic acid techniques to express an antibody in which the amino acid composition of the non-variable region is based on human antibodies. Computer programs have been designed to identify such non-variable regions.

As used herein, diabodies are dimeric scFV; diabodies typically have shorter peptide linkers than scFvs, and they generally dimerize.

As used herein, production by recombinant means by using recombinant DNA methods means the use of the well known methods of molecular biology for expressing proteins encoded by cloned DNA.

As used herein the term assessing is intended to include quantitative and qualitative determination in the sense of obtaining an absolute value for the activity of a SP, or a domain thereof, present in the sample, and also of obtaining an index, ratio, percentage, visual or other value indicative of the level of the activity. Assessment can be direct or indirect and the chemical species actually detected need not of course be the proteolysis product itself but can for example be a derivative thereof or some further substance.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein a biological activity of a protease its catalytic activity in which a polypeptide is hydrolyzed.

As used herein, functional activity refers to an activity or activities of a polypeptide or portion thereof associated with a full-length (complete) protein. Functional activities include, but are not limited to, biological activity, catalytic or enzymatic activity, antigenicity (ability to bind to or compete with a polypeptide for binding to an anti-polypeptide antibody), immunogenicity, ability to form multimers, and the ability to specifically bind to a receptor or ligand for the polypeptide.

As used herein, a conjugate refers to the compounds provided herein that include one or more SPs, including a CVSP16, particularly single chain protease domains thereof, and one or more targeting agents. These conjugates include those produced by recombinant means as fusion proteins, those produced by chemical means, such as by chemical coupling, through, for example, coupling to sulfhydryl groups, and those produced by any other method whereby at least one SP, or a domain thereof, is linked, directly or indirectly via linker(s) to a targeting agent.

As used herein, a targeting agent, is any moiety, such as a protein or effective portion thereof, that provides specific binding of the conjugate to a cell surface receptor, which in some instances can internalize bound conjugates or portions thereof. A targeting agent also can be one that promotes or facilitates, for example, affinity isolation or purification of the conjugate; attachment of the conjugate to a surface; or detection of the conjugate or complexes containing the conjugate.

As used herein, an antibody conjugate refers to a conjugate in which the targeting agent is an antibody.

As used herein, derivative or analog of a molecule refers to a portion derived from or a modified version of the molecule.

As used herein, an effective amount of a compound for treating a particular disease is an amount that is sufficient to ameliorate, or in some manner reduce the symptoms associated with the disease. Such an amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms.

As used herein equivalent, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions (such as, but not limited to, conservative changes such as those set forth in Table 1, above) that do not substantially alter the activity or function of the protein or peptide. When equivalent refers to a property, the property does not need to be present to the same extent (e.g., two peptides can exhibit different rates of the same type of enzymatic activity), but the activities are usually substantially the same. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically the two molecules are selected such that they will hybridize under conditions of high stringency.

As used herein, an agent that modulates the activity of a protein or expression of a gene or nucleic acid either decreases or increases or otherwise alters the activity of the protein or, in some manner, up- or down-regulates or otherwise alters expression of the nucleic acid in a cell.

As used herein, inhibitor of the activity of a SP encompasses any substances that prohibit or decrease production, post-translational modification(s), maturation, or membrane localization of the SP or any substance that interferes with or decreases the proteolytic efficacy thereof, particularly of a single chain form in an in vitro screening assay.

As used herein, a method for treating or preventing neoplastic disease means that any of the symptoms, such as the tumor, metastasis thereof, the vascularization of the tumors or other parameters by which the disease is characterized are reduced, ameliorated, prevented, placed in a state of remission, or maintained in a state of remission. It also means that the hallmarks of neoplastic disease and metastasis can be eliminated, reduced or prevented by the treatment. Non-limiting examples of the hallmarks include uncontrolled degradation of the basement membrane and proximal extracellular matrix, migration, division, and organization of the endothelial cells into new functioning capillaries, and the persistence of such functioning capillaries.

As used herein, pharmaceutically acceptable salts, esters or other derivatives of the conjugates include any salts, esters or derivatives that can be readily prepared by those of skill in this art using known methods for such derivatization and that produce compounds that can be administered to animals or humans without substantial toxic effects and that either are pharmaceutically active or are prodrugs.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound is regenerated by metabolic processes. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

As used herein, a drug identified by the screening methods provided herein refers to any compound that is a candidate for use as a therapeutic or as a lead compound for the design of a therapeutic. Such compounds can be small molecules, including small organic molecules, peptides, peptide mimetics, antisense molecules or dsRNA, such as RNAi, antibodies, fragments of antibodies, recombinant antibodies and other such compound which can serve as drug candidate or lead compound.

As used herein, a peptidomimetic is a compound that mimics the conformation and certain stereochemical features of the biologically active form of a particular peptide. In general, peptidomimetics are designed to mimic certain desirable properties of a compound, but not the undesirable properties, such as flexibility, that lead to a loss of a biologically active conformation and bond breakdown. Peptidomimetics can be prepared from biologically active compounds by replacing certain groups or bonds that contribute to the undesirable properties with bioisosteres. Bioisosteres are known to those of skill in the art. For example the methylene bioisostere CH₂S has been used as an amide replacement in enkephalin analogs (see, e.g., Spatola (1983) pp. 267-357 in Chemistry and Biochemistry of Amino Acids, Peptides, and Proteins, Weinstein, Ed. volume 7, Marcel Dekker, New York). Morphine, which can be administered orally, is a compound that is a peptidomimetic of the peptide endorphin. For purposes herein, cyclic peptides are included among peptidomimetics.

As used herein, a promoter region or promoter element refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences can be cis acting or can be responsive to trans acting factors. Promoters, depending upon the nature of the regulation, can be constitutive or regulated. Exemplary promoters contemplated for use in prokaryotes include the bacteriophage T7 and T3 promoters.

As used herein, a receptor refers to a molecule that has an affinity for a given ligand. Receptors can be naturally-occurring or synthetic molecules. Receptors also can be referred to in the art as anti-ligands. As used herein, the receptor and anti-ligand are interchangeable. Receptors can be used in their unaltered state or bound to other polypeptides, including as homodimers. Receptors can be attached to, covalently or noncovalently, or in physical contact with, a binding member, either directly or indirectly via a specific binding substance or linker. Examples of receptors, include, but are not limited to: antibodies, cell membrane receptors surface receptors and internalizing receptors, monoclonal antibodies and antisera reactive with specific antigenic determinants [such as on viruses, cells, or other materials], drugs, polynucleotides, nucleic acids, peptides, cofactors, lectins, sugars, polysaccharides, cells, cellular membranes, and organelles.

Examples of receptors and applications using such receptors, include but are not restricted to:
a) enzymes: specific transport proteins or enzymes essential to survival of microorganisms, which could serve as targets for antibiotic [ligand] selection;
b) antibodies: identification of a ligand-binding site on the antibody molecule that combines with the epitope of an antigen of interest can be investigated; determination of a sequence that mimics an antigenic epitope can lead to the development of vaccines of which the immunogen is based on one or more of such sequences or lead to the development of related diagnostic agents or compounds useful in therapeutic treatments such as for auto-immune diseases
c) nucleic acids: identification of ligand, such as protein or RNA, binding sites;
d) catalytic polypeptides: polymers, including polypeptides, that are capable of promoting a chemical reaction involving the conversion of one or more reactants to one or more products; such polypeptides generally include a binding site specific for at least one reactant or reaction intermediate and an active functionality proximate to the binding site, in which the functionality is capable of chemically modifying the bound reactant (see, e.g., U.S. Pat. No. 5,215,899);
e) hormone receptors: determination of the ligands that bind with high affinity to a receptor is useful in the development of hormone replacement therapies; for example, identification of ligands that bind to such receptors can lead to the development of drugs to control blood pressure; and
f) opiate receptors: determination of ligands that bind to the opiate receptors in the brain is useful in the development of less-addictive replacements for morphine and related drugs.

As used herein, sample refers to anything which can contain an analyte for which an analyte assay is desired. The sample can be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein: stringency of hybridization in determining percentage mismatch is as follows:
1) high stringency: 0.1×SSPE, 0.1% SDS, 65° C.
2) medium stringency: 0.2×SSPE, 0.1% SDS, 50° C.
3) low stringency: 1.0×SSPE, 0.1% SDS, 50° C.

Those of skill in this art know that the washing step selects for stable hybrids and also know the ingredients of SSPE (see, e.g., Sambrook, E. F. Fritsch, T. Maniatis, in: Molecular Cloning, A Laboratory Manual, Cold Spring Harbor Laboratory Press (1989), vol. 3, p. B.13, see, also, numerous catalogs that describe commonly used laboratory solutions). SSPE is pH 7.4 phosphate-buffered 0.18 M NaCl. Further, those of skill in the art recognize that the stability of hybrids is determined by Tₘ, which is a function of the sodium ion concentration and temperature (Tₘ=81.5° C.−16.6(log₁₀[Na⁺])+0.41(% G+C)−600/l)), so that the only parameters in the wash conditions critical to hybrid stability are sodium ion concentration in the SSPE (or SSC) and temperature.

It is understood that equivalent stringencies can be achieved using alternative buffers, salts and temperatures. By way of example and not limitation, procedures using conditions of low stringency are as follows (see also Shilo and Weinberg, Proc. Natl. Acad. Sci. USA 78:6789-6792 (1981)): Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA (10×SSC is 1.5 M sodium chloride, and 0.15 M sodium citrate, adjusted to a pH of 7).

Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Filters are blotted dry and exposed for autoradiography. If necessary, filters are washed for a third time at 65-68° C. and reexposed to film. Other conditions of low stringency which can be used are well known in the art (e.g., as employed for cross-species hybridizations).

By way of example and not way of limitation, procedures using conditions of moderate stringency include, for example, but are not limited to, procedures using such conditions of moderate stringency are as follows: Filters containing DNA are pretreated for 6 hours at 55° C. in a solution containing 6×SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 hours at 55° C., and then washed twice for 30 minutes at 60° C. in a solution containing 1×SSC and 0.1% SDS. Filters are blotted dry and exposed for autoradiography. Other conditions of moderate stringency which can be used are well-known in the art. Washing of filters is done at 37° C. for 1 hour in a solution containing 2×SSC, 0.1% SDS.

By way of example and not way of limitation, procedures using conditions of high stringency are as follows: Prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 minutes before autoradiography. Other conditions of high stringency which can be used are well known in the art.

The term substantially identical or homologous or similar varies with the context as understood by those skilled in the relevant art and generally means at least 60% or 70%, preferably means at least 80%, more preferably at least 90%, and most preferably at least 95% identity.

As used herein, substantially identical to a product means sufficiently similar so that the property of interest is sufficiently unchanged so that the substantially identical product can be used in place of the product.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

As used herein, target cell refers to a cell that expresses a SP in vivo.

As used herein, test substance (or test compound) refers to a chemically defined compound (e.g., organic molecules, inorganic molecules, organic/inorganic molecules, proteins, peptides, nucleic acids, oligonucleotides, lipids, polysaccharides, saccharides, or hybrids among these molecules such as glycoproteins, and others) or mixtures of compounds (e.g., a library of test compounds, natural extracts or culture supernatants and others) whose effect on a SP, particularly a single chain form that includes the protease domain or a sufficient portion thereof for activity, as determined by an in vitro method, such as the assays provided herein, is tested.

As used herein, a molecule, such as an antibody, that specifically binds to a polypeptide typically has a binding affinity (Kₐ) of at least about 10⁶ l/mol, 10⁷ l/mol, 10⁸ l/mol, 10⁹ l/mol, 10¹⁰ l/mol or greater and binds to a protein of interest generally with at least 2-fold, 5-fold, generally 10-fold or even 100-fold or greater, affinity than to other proteins. For example, an antibody that specifically binds to the protease domain compared to the full-length molecule, such as the zymogen form, binds with at least about 2-fold, typically 5-fold or 10-fold higher affinity, to a polypeptide that contains only the protease domain than to the zymogen form of the full-length. Such specific binding also is referred to as selective binding. Thus, specific or selective binding refers to greater binding affinity (generally at least 2-fold, 5-fold, 10-fold or more) to a targeted site or locus compared to a non-targeted site or locus.

As used herein, the terms a therapeutic agent, therapeutic regimen, radioprotectant, or chemotherapeutic mean conventional drugs and drug therapies, including vaccines, which are known to those skilled in the art. Radiotherapeutic agents are well known in the art.

As used herein, treatment means any manner in which the symptoms of a condition, disorder or disease are ameliorated or otherwise beneficially altered. Treatment also encompasses any pharmaceutical use of the compositions herein.

As used herein, vector (or plasmid) refers to discrete elements that are used to introduce heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art. An expression vector includes vectors capable of expressing DNA that is operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, protein binding sequence refers to a protein or peptide sequence that is capable of specific binding to other protein or peptide sequences generally, to a set of protein or peptide sequences or to a particular protein or peptide sequence.

As used herein, epitope tag refers to a short stretch of amino acid residues corresponding to an epitope to facilitate subsequent biochemical and immunological analysis of the epitope tagged protein or peptide. Epitope tagging is achieved by adding the sequence of the epitope tag to a protein-encoding sequence in an appropriate expression vector. Epitope tagged proteins can be affinity purified using highly specific antibodies raised against the tags.

As used herein, metal binding sequence refers to a protein or peptide sequence that is capable of specific binding to metal ions generally, to a set of metal ions or to a particular metal ion.

As used herein, a combination refers to any association between two or among more items.

As used herein, a composition refers to any mixture. It can be a solution, a suspension, liquid, powder, a paste, aqueous, non-aqueous or any combination thereof.

As used herein, fluid refers to any composition that can flow. Fluids thus encompass compositions that are in the form of semi-solids, pastes, solutions, aqueous mixtures, gels, lotions, creams and other such compositions.

As used herein, a kit is a packaged combination optionally including instructions for use of the combination and/or other reactions and components for such use.

As used herein, a cellular extract refers to a preparation or fraction which is made from a lysed or disrupted cell.

As used herein, an agent is said to be randomly selected when the agent is chosen randomly without considering the specific sequences involved in the association of a protein alone or with its associated substrates, binding partners and/or other components. An example of randomly selected agents is the use a chemical library or a peptide combinatorial library, or a growth broth of an organism or conditioned medium.

As used herein, an agent is said to be rationally selected or designed when the agent is chosen on a non-random basis which takes into account the sequence of the target site and/or its conformation in connection with the agent's action. As described in the Examples, there are proposed binding sites for serine protease and (catalytic) sites in the protein having SEQ ID No. 6. Agents can be rationally selected or rationally designed by using the peptide sequences that make up these sites.

For clarity of disclosure, and not by way of limitation, the detailed description is divided into the subsections that follow.

B. CVSP16 Polypeptides, Muteins, Derivatives and Analogs Thereof

SPs

The serine proteases (SPs) are a family of proteins found in mammals and also other species. SPs share a number of common structural features as described herein. The proteolytic domains share sequence homology generally including conserved His, Asp, and Ser residues necessary for efficient catalytic activity that are present in conserved motifs. These SPs generally are synthesized as zymogens, and activated to multi-chain forms (i.e., two-chains, three-chains) by specific cleavage.

The SP family can be targeted for therapeutic intervention and also can serve as diagnostic markers for tumor initiation, development, growth and/or progression. As discussed, members of this family are involved in proteolytic processes that are implicated in tumor development, growth and/or progression. This implication is based upon their functions as proteolytic enzymes in extracellular matrix degradation and remodelling and growth- and pro-angiogenic factor activation. In addition, their levels of expression or level of activation or their apparent activity resulting from substrate and/or co-factor levels or alterations in substrates and/or co-factors and levels thereof differ in tumor cells from non-tumor cells in the same tissue. Hence, protocols and treatments that alter their activity, such as their proteolytic activities and roles in signal transduction, and/or their expression, such as by contacting them with a compound that modulates their activity and/or expression, could impact tumor development, growth and/or progression. Also, in some instances, the level of activation and/or expression can be altered in tumors, such as pancreas, stomach, uterus, lung, colon and cervical cancers, and also breast, prostate or leukemias. The SP, thus, can serve as a diagnostic marker for tumors.

In other instances the SP protein can exhibit altered activity by virtue of a change in activity or expression of a co-factor therefor or a substrate therefor. Detection of the SPs, particularly the protease domains, in body fluids, such as serum, blood, saliva, cerebral spinal fluid, synovial fluid and interstitial fluids, urine, sweat and other such fluids and secretions, can serve as a diagnostic tumor marker. In particular, detection of higher levels of such polypeptides in a subject compared to a subject known not to have any neoplastic disease or compared to earlier samples from the same subject, can be indicative of neoplastic disease in the subject.

CVSP16

Provided are polypeptides family members designated CVSP16. The CVSP16s provided herein are serine proteases that are expressed and/or activated in certain tumors; hence their activation or expression can serve as a diagnostic marker for tumor development, growth and/or progression. The CVSP16 polypeptides provided herein and can be used as a drug or therapeutic target and used in screening assays, including those exemplified herein. Dimerized and higher multimers of CVSP16 polypeptides and/or portions thereof are provided.

A single chain proteolytic domain can function in vitro and, hence is useful in in vitro assays for identifying agents that modulate the activity of members of this family. In addition two-chain or three chain forms, the activated full-length or truncated forms thereof, such as forms in which the signal peptide is removed, also can be used in such assays. Assays for activation also are provided.

The CVSP16 polypeptides provided herein are expressed or activated or active in tumor cells, typically at a level that differs from the level in which they are expressed or activated or active in the non-tumor cell of the same type. Hence, for example, if the CVSP16 is expressed in a prostate or ovarian tumor cell, to be of interest herein with respect to ovarian or prostate cancer, it is expressed or activated or active at a different level in non-tumor prostate or ovarian cells. CVSP16 polypeptide is expressed in lung, colon, prostate, breast, uterine, ovarian and other tumor cells.

As discussed, the members of this family are involved in proteolytic processes that are implicated in tumor development, growth and/or progression. This implication is based upon their functions as proteolytic enzymes in processes related to ECM degradative pathways. In addition, their levels of expression or level of activation or their apparent activity resulting from substrate levels or alterations in substrates and levels thereof differs in tumor cells and non-tumor cells in the same tissue. Also, in some instances, the level of activation and/or expression can be altered in tumors, such as pancreas, stomach, uterus, lung, colon and cervical cancers, and also breast, prostate or leukemias. The SP, thus, can serve as a diagnostic marker for tumors.

It is shown herein, that CVSP16s provided herein are expressed and/or activated in certain tumors; hence their activation or expression can serve as a diagnostic marker for tumor development, growth and/or progression. The CVSP16 also is intended for use as a drug target and used in screening assays, including those CSVP16s exemplified herein. The CVSP16 polypeptides provided herein are expressed or activated by or in tumor cells, typically at a level that differs from the level in which they are expressed by the non-tumor cell of the same type. Hence, for example, if the SP expressed by a prostate or ovarian tumor cell is to be of interest herein with respect to ovarian or prostate cancer, it should have an expression, extent of activation or activity that is different from that in non-tumor cells. CVSP16 is expressed in lung, colon, prostate, breast, uterine, ovarian and other tumor cells.

In certain embodiments, a CVSP16 polypeptide or a portion thereof is detectable in a body fluid at a level that differs from its level in body fluids in a subject not having a tumor. In other embodiments, the polypeptide is present in a tumor; and a substrate or cofactor for the polypeptide is expressed at levels that differ from its level of expression in a non-tumor cell in the same type of tissue.

Detection of the SP, or a protease domain thereof, in body fluids, such as serum, blood, saliva, cerebral spinal fluid, synovial fluid and interstitial fluids, urine, sweat and other such fluids and secretions, can serve as a diagnostic tumor marker. In particular, detection of higher levels or alternative forms of such polypeptides in a subject compared to a subject known not to have any neoplastic disease or compared to earlier samples from the same subject, can be indicative of neoplastic disease in the subject.

Isolated, substantially pure proteases that contain protease domains or catalytically active (or functionally active) portions thereof in a single chain form of SPs are provided. A protease domain or plurality thereof can be included in a longer protein, and such longer protein is optionally the CVSP16 zymogen. In particular, exemplary protease domains include at least a sufficient portion of sequences of amino acids set forth in SEQ ID No. 6 (encoded by nucleotides in SEQ ID No. 5) to exhibit protease activity in an assay provided herein.

The domains, fragments, derivatives or analogs of a CVSP16 that are functionally active are capable of exhibiting one or more functional activities associated with the CVSP16 polypeptide, such as serine protease activity, immunogenicity and antigenicity, are provided. CVSP16 contains a signal peptide sequence (aa 1 to aa 23) and two protease domains. Also included are substantially purified CVSP16 zymogen, activated multi-chain forms of the polypeptide, single chain protease domains, two-chain protease domains. These are encoded by a nucleic acid that includes sequence encoding at least one protease domain that exhibits proteolytic activity and that hybridizes to a nucleic acid molecule including the sequence of nucleotides set forth in SEQ ID No. 5, typically under moderate, generally under high stringency, conditions and generally along the full length (or at least about 70, 80 or 90%) of a protease domain. Polypeptides encoded by splice variants are also provided as long as the polypeptides do not include at least 5, 7, 10, 15, 20 or more contiguous amino acids set forth in SEQ ID No. 21.

CVSP16 Polypeptides

As described CVSP16 polypeptides provided herein include polypeptides:
a) a polypeptide encoded by the sequence of nucleotides set forth in SEQ ID No. 5;
b) a polypeptide encoded by a sequence of nucleotides that hybridizes under conditions of low, moderate or high stringency to the sequence of nucleotides set forth in SEQ ID No. 5, wherein the encoded polypeptide does not include at least 5 or at least 6 or at least 7 or at least 8 contiguous or at least 9 or at least 10 contiguous amino acids from SEQ ID No. 21 up to all of SEQ ID No. 21, and particularly do not include any amino acids therefrom between amino acids corresponding to Gln₆₆₀ and Met₆₆₁ of SEQ ID No. 6;
c) a polypeptide that contains the sequence of amino acids set forth in SEQ ID No. 6, particularly, amino acids 24-752 and C-terminal truncated portions thereof;
d) a polyeptide that contains at least two protease domains of a serine protease 16 (CVSP16) and includes at least 5 contiguous amino acids corresponding to residues 508-544 of SEQ ID No. 6. or contains the contiguous sequence Asn Asp Ser or Trp Asn Asp or Ser Cys Trp Asn Asp Ser or Cys Trp Asn Asp Ser or GIn Thr His or Leu Gin Thr His in the second protease domain;
e) a polypeptide that contains a sequence of amino acids having at least about 60%, 70%, 80%, 90% or about 95% sequence identity with the sequence of amino acids set forth in SEQ ID No. 6, wherein the polypeptide does not include at least 5 or at least 6 or at least 7 or at least 8 contiguous or at least 9 or at least 10 contiguous amino acids from SEQ ID No. 21 up to all of SEQ ID No. 21, and particularly does not include any amino acids therefrom between amino acids corresponding to Gln₆₆₀ and Met₆₆₁ of SEQ ID No. 6;
f) the polypeptide is encoded by a sequence of nucleotides that hybridizes under conditions of at least moderate, and can be high, stringency along at least 70% of its full length to a sequence of nucleotides than encodes a polypeptide of any of a)-e), wherein the polypeptide does not include at least 5 or at least 6 or at least 7 or at least 8 contiguous or at least 9 or at least 10 contiguous amino acids from SEQ ID No. 21 up to all of SEQ ID No. 21, and particularly do not include any amino acids therefrom between amino acids corresponding to Gln₆₆₀ and Met₆₆₁ of SEQ ID No. 6;
g) the polypeptide has at least 60%, 70%, 80%, 90% or about 95% sequence identity with a polypeptide of any of a)-f), wherein the polypeptide does not include at least 5 or at least 6 or at least 7 or at least 8 contiguous or at least 9 or at least 10 contiguous amino acids from SEQ ID No. 21 up to all of SEQ ID No. 21, and particularly do not include any amino acids therefrom between amino acids corresponding to Gln₆₆₀ and Met₆₆₁ of SEQ ID No. 6; and/or
h) a polypeptide encoded by a splice variant of a sequence of nucleotides that encodes a CVSP16 polypeptide provided herein. Smaller portions thereof that retain protease activity are contemplated.

An exemplary CVSP16 polypeptide includes the sequence of amino acids set forth in SEQ ID No. 6; the protease domains are included in the sequence of amino acids set forth in SEQ ID No. 6.

Also, provided herein are isolated substantially pure polypeptides that contain a protease domain of a CVSP16. The polypeptide protein also can include other non-CVSP16 sequences of amino acids, but includes a protease domain or a sufficient portion thereof to exhibit catalytic activity (or other functional activity, such as substrate or ligand binding activity) in any in vitro assay that assess such protease activity, such as any provided herein. The CVSP16 polypeptides do not include the sequence of amino acids set forth in SEQ ID No. 21 and/or they do not include at least 5, 7, 9, 10, 11, 15, 20 or more contiguous amino acids thereof between amino acids that correspond to Q660 and M661 of SEQ ID No. 6 and/or the CVSP16 polypeptide contains at least two protease domains of a serine protease 16 (CVSP16) and includes at least 5 contiguous amino acids corresponding to residues 508-544 of SEQ ID No. 6. (SEQ ID No. 22) or contains the contiguous sequence Asn Asp Ser or Trp Asn Asp or Ser Cys Trp Asn Asp Ser or Cys Trp Asn Asp Ser or Gln Thr His or Leu Gln Thr His in the second protease domain. Such polypeptides are zymogens or activated single- two- or three-chain molecules.

Provided are substantially purified CVSP16 zymogens, activated two and three chain forms, single chain protease domains and two-chain protease domains. A full-length exemplary CVSP16 polypeptide, including the signal sequence, is set forth in SEQ ID No. 6 The signal sequence (see amino acids 1-23, SEQ ID No. 6) can be cleaved upon expression or the encoding nucleic acid can be deleted prior to expression.

Also provided is a substantially purified protein including a sequence of amino acids that has at least 60%, 70%, 80%, 90% or about 95%, identity to the exemplified CVSP16 or to a protease domain thereof, and does not include at least 5, 7, 9, 11, 15, 20 or more contiguous amino acids of SEQ ID No. 21. Percentage identity is determined using standard algorithms and gap penalties that maximize the percentage identity. A huma CVSP16 polypeptide is exemplified, although other mammalia CVSP16 polypeptides are contemplated and are obtained by standard methods using the disclosed CVSP16-encoding nucleic acid (or antibodies made to the CVSP16) to isolate corresponding nucleic acid molecules (and/or CVSP16s) from other species. Polypeptides peptides encoded by splice variants of the exemplified encoding nucleic acid (SEQ ID No. 5), particularly those with a proteolytically active protease domain, but not containing at least 5, 7, 10, 15, 20 or more contiguous amino acids from SEQ ID No. 21, particularly inserted between the amino acids corresponding to Q660 and M661 (SEQ ID No. 6) are provided.

Provided are substantially purified CVSP16 polypeptides and functional domains thereof, including catalytically active domains and portions, that have at least about 60%, 70%, 80%, 90% or about 95% sequence identity with a CVSP16 that includes the sequence of amino acids set forth in SEQ ID No. 6 or a catalytically active portion thereof. The CVSP16 polyeptides provided herein do not include at least 5 contiguous amino acid residues as set forth in SEQ ID No. 21, particularly between residues corresponding to Q₆₆₀ and M₆₆₁ of SEQ ID No. 6. Also provided are polypeptides that are encoded by the nucleic acid molecules provided herein. Polypeptides that contain at least two protease domains of a serine protease 16 (CVSP16) and includes at least 5 contiguous amino acids corresponding to residues 508-544 of SEQ ID No. 6. or contains the contiguous sequence Asn Asp Ser or Trp Asn Asp or Ser Cys Trp Asn Asp Ser or Cys Trp Asn Asp Ser or Gln Thr His or Leu Gln Thr His in the second protease domain.

In other embodiments, substantially purified polypeptides that include a protease domain of a CVSP16 polypeptide or a catalytically active portion thereof, but that do not include the entire sequence of amino acids set forth in SEQ ID No. 6 are provided. Among these are polypeptides that include a sequence of amino acids that has at least 60%, 70%, 80%, 90%, 95% or 100% sequence identity to SEQ ID No. 6.

Included among the polypeptides provided herein is a CVSP16 protease domain and/or a polypeptide with amino acid changes such that the specificity and protease activity (or other functional activity) is not eliminated and is retained at least 1%, 2%, 3%, 5%, 10%, 20%, 30%, 40%, 50% activity or remains substantially unchanged (more than about 50%) or increases. The CVSP16 polypeptide can form homodimers and also can form heterodimers with some other protein, such as a membrane-bound protein.

Domains, fragments, derivatives or analogs of a CVSP16 that are functionally active are capable of exhibiting one or more functional activities and/or other activities or properties, such as immunogenicity and antigenicity, also are provided.

### Protease Domains and Antigenic Fragments

Isolated, substantially pure polypeptides that include the protease domains or catalytically active portions thereof as single chain forms of SPs are provided. The protease domains can be included in a longer protein, and such longer proteins include up to a full-length CVSP16 zymogen as long as the full-length polypeptide does not include SEQ ID No. 21. Provided herein are isolated substantially pure polypeptides that contain a protease domain of a CVSP16 as a single chain.

SP protease domains include the single-chain protease domains of CVSP16. Provided are the protease domains or proteins that include a portion of a SP that is the protease domain of any SP, particularly a CVSP16. The protein also can include other non-SP sequences of amino acids, but includes the protease domain or a sufficient portion thereof to exhibit catalytic and/or binding activity in any in vitro assay that assesses such activity(ies), such as any provided herein. Also provided are two- and three-chain activated forms of the full length protease and also two chain forms of a protease domain.

In an embodiment, the substantially purified CVSP16 protease is encoded by a nucleic acid that hybridizes to a nucleic acid molecule containing the protease domain encoded by the nucleotide sequence set forth in SEQ. ID No. 5 under at least moderate, generally high, stringency conditions, such that the protease domain encoding nucleic acid thereof hybridizes along its full length or along at least about 70%, 80% or 90% of the full length. In other embodiments the substantially purified CVSP16 protease is a single chain polypeptide that includes substantially the sequence of amino acids set forth as amino acids 24-752 in SEQ ID No. 6, or a catalytically active (or functionally active or immunogenic or antigenic) portion thereof.

Polypeptides that additionally include amino acids at the C-terminus, such as all or a portion of the amino acids following the protease domain (aa 551 to aa 752 in SEQ ID No. 6) in the exemplified embodiment are provided. Dimers and other multimers of the full length and catalytically active portions of the polypeptides that include PD1 and/or PD2 are provided.

A signal peptide (amino acids 1-23 of SEQ ID No. 6 in the exemplified embodiment) also is provided. In addition the mature CVSP16 polypeptide with the signal sequence removed and catalytically active portions thereof, including those that include all or a portion of the C-terminus beyond the protease domain are provided.

As described below, all forms of the CVSP16, including the pro-polypeptide with the signal sequence, the mature polypeptide and catalytically active portions thereof, the protease domains and catalytically active portions thereof, three-chain, two-chain and single chain forms of any of these polypeptides are provided herein and can be used in the screening assays and for preparing specific antibodies therefor. The expression, quantity, activity and/or activation of the polypeptides in tumor cells and body fluids can be diagnostic of disease or its absence.

CVSP16 has two protease domains PD1 and PD2 (see, e.g., amino acids 46 to 286 an amino acids 323 to 550 of SEQ ID No. 6, respectively). Polypeptides that contain PD1 or PD2 as the only CVSP16 portion thereof are provided or that contain PD1 and PD2 but do not include at least 5, 7, 10, 15, 20 or more contiguous amino acids of SEQ ID No. 21 are provided.

With reference to SEQ ID No. 6 of the exemplified CVSP16, the exemplified CVSP16 has 8 putative N-linked glycosylation sites ( . . . N₉₂GT . . . , . . . N₁₃₀YS . . . , . . . N₂₁₇LT . . . , . . . N₃₁₇CT . . . , . . . N₃₆₉SS . . . , . . . N₄₀₂AS . . . , . . . N₄₂₁LS . . . , . . . N₅₀₈DS . . . ). The following cysteine pairings are noted: C₇₂-C₈₈, C₁₇₃-C₂₄₉, C₂₀₆-C₂₂₈, C₂₃₉-C₂₆₇, C₃₄₈-C₃₆₄, C₄₄₄-C₅₁₆, C₄₇₂-C₄₉₄ and C₅₀₆-C₅₃₄. In addition, an unpaired cysteine (C₁₅₉) in the first protease domain should pair with C₃₈. An unpaired cysteine (C₄₃₀) in the second protease domain should pair with C₃₂₅ or C₃₁₈. As a result the protease, upon activation cleavage can contain multiple chains, including two or three or more chains, formed by virtue of pairing between the unpaired cysteine in a protease domain with a cysteine outside a protease domain.

The first protease domain is characterized by the presence of a protease activation cleavage site ( . . . R₄₆↓I₄₇VGGSNAQP . . . , where ↓ indicates protease activation cleavage site) at the beginning of the domain and catalytic triad residues (H₈₇, D₁₃₉ and S₂₄₃) in 3 highly-conserved regions of the catalytic domain. In the second protease domain, the invariant catalytic histidine is replaced by a serine (S₃₆₃) residue, and the highly conserved SGGP sequence that contains the catalytic serine is replaced with the sequence S₅₁₀RWS. In addition, PD2 begins at amino acid 323-324 (Pro₃₂₃ Glu₃₂₄), which provides an unusual cleavage site. Since Pro-Glu residues as a cleavage site is unusual, cleavage may not be needed for activation. These sequences and differences from other protease domains indicate that the second protease domain has lower catalytic activity.

Antigenic Epitopes

Antigenic epitopes that contain at least 4, 5, 6, 7, 8, 9, 10, 11, 12, 13, 14, 15, 20, 25, 30, 40, 50, and typically 10-15 amino acids of the CVSP16 polypeptide are provided. These antigenic epitopes are used, for example, to raise antibodies. Antibodies, see further discussion below, specific for each epitope or combinations thereof are also provided. Also provided are antibodies that bind with at least 10-fold or 100-fold greater affinity to CVSP16 polypeptides that do not include the sequence of amino acids set forth in SEQ ID No. 21 (particularly do not include any amino acids therefrom between amino acids corresponding to Gln₆₆₀ and Met₆₆₁ of SEQ ID No. 6) compared to those that include SEQ ID No. 21. In particular, provided are antibodies that bind to a CVSP16 polypeptide provided herein that does not include at least 5, 7, 9, 10, 15, 20 contiguous amino acids of SEQ ID No. 21, particularly where the contiguous amino acids are inserted between amino acids corresponding to Q₆₆₀ and M₆₆₁, with at least 2-, 5-, 10-, 100-fold greater affinity than to a polypeptide that includes the least 5 contiguous amino acids set forth in SEQ ID No. 21, particularly where the continuous amino acids are inserted between amino acids corresponding to Q₆₆₀ and M₆₆₁.

### Muteins and Derivatives of CVSP16 Polypeptides

Full-length CVSP16, zymogen and activated forms thereof and CVSP16 protease domains, portions thereof, and muteins and derivatives of such polypeptides are provided. Among the derivatives are those based on animal CVSP16s, including, but are not limited to, rodent, such as mouse and rat; fowl, such as chicken; ruminants, such as goats, cows, deer, sheep; ovine, such as pigs; and humans. For example, CVSP16 derivatives can be made by altering their sequences by substitutions, additions or deletions. CVSP16 derivatives include, but are not limited to, those containing, as a primary amino acid sequence, all or part of the amino acid sequence of CVSP16, including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence can be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid (see, e.g., Table 1). Muteins of the CVSP16 or a domain thereof, such as a protease domain, in which up to about 10%, 20%, 30%, 40%, 50%, 60%, 70%, 80%, 85%, 90% or 95% of the amino acids are replaced with another amino acid are provided. Generally such muteins retain at least about 1%, 2%, 3,%, 5%, 7%, 8%, 10%, 20%, 30%, 40%, 50%, 60%, 70%, 80%, 90%, 95% or more (or in increased activity, i.e., 101, 102, 103, 104, 105, 110% or greater) of the protease activity of the unmutated protein. Those of skill in the art recognize that a polypeptide that retains at least 1% of the activity of the wild-type protease is sufficiently active for use in screening assays or in other applications.

CVSP16 derivatives can be made by altering their sequences by substitutions, additions or deletions that provide for functionally equivalent molecules. Due to the degeneracy of nucleotide coding sequences, other nucleic sequences which encode substantially the same amino acid sequence as a CVSP16 are provided. These include but are not limited to nucleotide sequences containing all or portions of CVSP16 genes that are altered by the substitution of different codons that encode the amino acid residue within the sequence, thus producing a silent change. CVSP16 derivatives include, but are not limited to, those containing, as a primary amino acid sequence, all or part of the amino acid sequence of SP, including altered sequences in which amino acid residues are substituted for residues within the sequence resulting in a silent change or a change that eliminates an activity. Substitutes for an amino acid within the sequence can be selected from other members of the class to which the amino acid belongs or from another class (for non-conservative changes).

Muteins in which one or more of the Cys residues, particularly, a residue that is paired in the activated two-chain or three-chain form, but unpaired in the protease domain alone (i.e., a Cys at residue no. 159 and/or 430 in SEQ ID No. 6) is/are replaced with any amino acid, typically, although not necessarily, a conservative amino acid residue, such as Ser, are contemplated. Muteins in which 10%, 20%, 30%, 35%, 40%, 45%, 50% or more of the amino acids are replaced but the resulting polypeptide retains at least about 1%, 3%, 5%, 10%, 20%, 30%, 35%, 40%, 45%, 50%, 60%, 70%, 80%, 90% or 95% of the catalytic activity (or other functional activity or other property, such as antigenicity or immunogenicity) as the unmodified form for the same substrate also are provided.

### Nucleic Acid Molecules, Vectors and Plasmids, Cells and Expression of CVSP16 Polypeptides

Also provided herein are nucleic acid molecules that encode SP proteins and the encoded proteins. In particular, nucleic acid molecules encoding CVSP16 from animals, including splice variants thereof are provided. The encoded proteins are also provided. Also provided are functional domains thereof. For example, the SP (CVSP16) protease domains, portions thereof, and muteins thereof are from or based on animal SPs (CVSPs), including, but are not limited to, rodent, such as mouse and rat; fowl, such as chicken; ruminants, such as goats, cows, deer, sheep; ovine, such as pigs; and humans. The isolated nucleic acid fragment is DNA, including genomic or cDNA, or is RNA, or can include other components, such as PNA. The isolated nucleic acid can include additional components, such as heterologous or native promoters, and other transcriptional and translational regulatory sequences, these genes can be linked to other genes, such as reporter genes or other indicator genes or genes that encode indicators.

### Nucleic Acid Molecules

Due to the degeneracy of nucleotide coding sequences, other nucleic acid sequences that encode substantially the same amino acid sequence as CVSP16 gene (or cDNA or RNA) can be used. These include but are not limited to nucleotide sequences comprising all or portions of CVSP16 genes that are altered by the substitution of different codons that encode the amino acid residue within the sequence, thus producing a silent change.

Also provided are nucleic acid molecules that hybridize to the above-noted sequences of nucleotides encoding CVSP16 at least at low stringency, at moderate stringency, and/or at high stringency, and that encode the protease domain and/or the full length protein or other domains of a CVSP16 or a splice variant or allelic variant thereof. Generally the molecules hybridize under such conditions along their full length (or along at least about 70%, 80% or 90% of the full length) for at least one domain and encode at least one domain, such as the protease domain, of the polypeptide. In particular, such nucleic acid molecules include any isolated nucleic acid fragment that encodes at least one domain of a serine protease, that (1) contains a sequence of nucleotides that encodes the protease or a functionally active, such as catalytically active, domain thereof, and (2) is selected from among sequences of nucleic acids that encode a CVSP16 polypeptide:
a) a polypeptide encoded by the sequence of nucleotides set forth in SEQ ID No. 5;
b) a polypeptide encoded by a sequence of nucleotides that hybridizes under conditions of low, moderate or high stringency to the sequence of nucleotides set forth in SEQ ID No. 5, wherein the encoded polypeptide does not include at least 5, 6, 7, 8, 9, 15 or 20 contiguous amino acids from SEQ ID No. 21 up to all of SEQ ID No. 21, and particularly do not include the contiguous amino acids therefrom between amino acids corresponding to Gln₆₆₀ and Met₆₆₁ of SEQ ID No. 6;
c) a polypeptide that contains the sequence of amino acids set forth in SEQ ID No. 6 or that contains amino acid residues 24-752 or functionally active, particularly, catalytically active, portions thereof;
d) a polypeptide that contains a sequence of amino acids having at least about 60%, 70%, 80%, 90% or about 95% sequence identity with the sequence of amino acids set forth in SEQ ID No. 6, where the polypeptide does not include at least at least 5, 6, 7, 8, 9, 10, 15 or 20 contiguous amino acids from SEQ ID No. 21 up to all of SEQ ID No. 21, and particularly do not include any amino acids therefrom between amino acids corresponding to Gln₆₆₀ and Met₆₆₁ of SEQ ID No. 6;
e) a polypeptide that contains at least two protease domains of a serine protease 16 (CVSP16) and includes at least 5 contiguous amino acids corresponding to residues 508-544 of SEQ ID No. 6. or contains the contiguous sequence Asn Asp Ser or Trp Asn Asp or Ser Cys Trp Asn Asp Ser or Cys Trp Asn Asp Ser or Gln Thr His or Leu Gln Thr His in the second protease domain;
f) a polypeptide encoded by a sequence of nucleotides that hybridizes under conditions of at least moderate, and can be high, stringency along at least 70% of its full length to a sequence of nucleotides that encodes a polypeptide of any of a)-e), wherein the polypeptide does not include at least 5, 6, 7, 8, 9, 10, 15 or 20 contiguous amino acids contiguous amino acids from SEQ ID No. 21 up to all of SEQ ID No. 21, and particularly does not include any amino acids therefrom between amino acids corresponding to Gln₆₆₀ and Met₆₆₁ of SEQ ID No. 6;
g) the polypeptide has at least 60%, 70%, 80%, 90% or about 95% sequence identity with a polypeptide of any of a)-), where the polypeptide does not include at least about 5, 6, 7, 8, 9, 10, 15 or 20 contiguous amino acids from SEQ ID No. 21 up to all of SEQ ID No. 21, and particularly do not include any amino acids therefrom between amino acids corresponding to Gln₆₆₀ and Met₆₆₁ of SEQ ID No. 6; and/or
h) a polypeptide encoded by a splice variant of a sequence of nucleotides that encodes a CVSP16 polypeptide, including a polypeptide of any of a)-f), as long as the resulting polypeptide the polypeptide does not include at least about 5, 6, 7, 8, 9, 10, 15 or 20 contiguous amino acids from SEQ ID No. 21 up to all of SEQ ID No. 21, and particularly does not include any amino acids therefrom between amino acids corresponding to Gln₆₆₀ and Met₆₆₁ of SEQ ID No. 6 and/or the polypeptide encoded by the splice variant contains at least two protease domains of a serine protease 16 (CVSP16) and includes at least 5 contiguous amino acids corresponding to residues 508-544 of SEQ ID No. 6. or contains the contiguous sequence Asn Asp Ser or Trp Asn Asp or Ser Cys Trp Asn Asp Ser or Cys Trp Asn Asp Ser or Gln Thr His or Leu Gin Thr His in the second protease domain. Smaller nucleic acid molecules that encode polypeptides that retain protease activity as single chains or as other truncated single-chain forms are provided.

Hence, among the nucleic acid molecules are those that do not encode a polypeptide that includes at least about 5, 6, 7, 8, 9, 10, 15 or 20 contiguous amino acids from SEQ ID No. 21 up to all of SEQ ID No. 21, and particularly do not include any amino acids therefrom between amino acids corresponding to Gln₆₆₀ and Met₆₆₁ of SEQ ID No. 6 and contain:
(a) a sequence of nucleotides that encodes the CVSP16 polypeptide or a domain thereof, particularly a CVSP16 polypeptide of a)-h) above;
(b) a sequence of nucleotides that encodes such portion or the full length CVSP16 protease and hybridizes under conditions of moderate or high stringency, generally to nucleic acid that is complementary to an mRNA transcript present in a mammalian cell that encodes a mammalian CVSP16 protein or a fragment thereof;
(c) a sequence of nucleotides that encodes a CVSP16 protease or a domain thereof that includes a sequence of amino acids encoded by such portion or the full length open reading frame; and
(d) a sequence of nucleotides that encodes a CVSP16 protease that includes a sequence of amino acids encoded by a sequence of nucleotides that encodes such protease and hybridizes under conditions of moderate or high stringency to DNA that is complementary to a mRNA transcript.
(e) a sequence of nucleotides that includes degenerate codons of all or a portion of any of (a)-(d).

The isolated nucleic acid fragment is DNA, including genomic or cDNA, or is RNA, or can include other components, such as peptide nucleic acid (PNA) or other modified nucleotides or ribonucleotides or analogs thereof. The isolated nucleic acid can include additional components, such as heterologous or native promoters, and other transcriptional and translational regulatory sequences, these genes can be linked to other genes, such as reporter genes or other indicator genes or genes that encode indicators.

In an exemplary embodiment, a nucleic acid molecule that encodes a CVSP16 polypeptide is provided. In particular, an encoding nucleic acid molecule with an open reading frame within the sequence of nucleotides set forth in SEQ ID No. 5 is provided. The encoded protein is set forth in SEQ ID NO. 6 (see, also EXAMPLE 1). Also provided a nucleic acid molecules that encode the mature polypeptide (residues 24-752) and one or both protease domains. The encoded polypeptide does not include at least about 5, 6, 7, 8, 9, 10, 15 or 20 contiguous amino acids from SEQ ID No. 21 up to all of SEQ ID No. 21, and particularly do not include any amino acids therefrom between amino acids corresponding to Gln₆₆₀ and Met₆₆₁ of SEQ ID No. 6.

Also provided are nucleic acid molecules that hybridize under conditions of at least low stringency, typically moderate stringency, and generally high stringency, and typically along at least 70%, 80%, 90% or 95% of their full length to the above sequence of nucleic acids (SEQ ID No. 5 or degenerates thereof), particularly to the open reading frame encompassed by nucleotides that encode a single protease domain or any domain of CVSP16, and/or to any of the CVSP16 polypeptides and fragments thereof as described herein.

Also provided are nucleic acid molecules that encode a single chain CVSP16 protease that has proteolytic activity in an in vitro proteolysis assay and that encode a polypeptide that has at least 80%, 85%, 90% or 95% sequence identity with the full length CVSP16 polypeptide of SEQ ID No. 6.

Also provided are nucleic acid molecules that encode a single chain CVSP16 protease that has proteolytic activity in an in vitro proteolysis assay and that encode a polypeptide that has at least 60%, 70%, 80%, 85%, 90% or 95% sequence identity with the full length of a protease domain of the CVSP16 polypeptide of SEQ ID No. 6, or that hybridize along their full length to a nucleic acids that encodes a protease domain, particularly under conditions of moderate, generally high, stringency. As above, the encoded polypeptides contain the protease as a single chain.

The isolated nucleic acids can include at least 8 nucleotides of a CVSP16-encoding sequence. In other embodiments, the nucleic acids can contain at least 10, 14, 16, 50, 100 nucleotides, 150 nucleotides, or 200 nucleotides of a CVSP16-encoding sequence provided that the nucleic acid molecules includes codons encoding Q660 and M661 as contiguous amino acids, particular those encoding amino acids 655-665 or smaller portions thereof that include Q₆₆₀ and M₆₆₁ of SEQ ID No. 6. The nucleic acid molecules generally include sequence of nucleotides that encode residues that correspond to Q₆₆₀ and M₆₆₁ of SEQ ID No. 6.

For each of the nucleic acid molecules, the nucleic acid can be DNA or RNA or PNA or other nucleic acid analogs or can include non-natural nucleotide bases. Also provided are isolated nucleic acid molecules that include a sequence of nucleotides complementary to the nucleotide sequence encoding a SP.

### Probes, Primers, Antisense Oligonucleotides and dsRNA

Also provided are fragments thereof or oligonucleotides that can be used as probes or primers and that contain at least about 10, 14, 16 nucleotides, generally less than 1000 or less than or equal to 100, set forth in SEQ ID No. 5 (or the complement thereof); or contain at least about 30 nucleotides (or the complement thereof) or contain oligonucleotides that hybridize along their full length or along at least about 70%, 80% or 90% of their full length to any such fragments or oligonucleotides. The length of the fragments is a function of the purpose for which they are used and/or the complexity of the genome of interest. Generally probes and primers contain less than about 500, 150, 100 nucleotides. The probes and primers generally span residues that correspond to Q₆₆₀ and M₆₆₁.

Probes and primers derived from the nucleic acid molecules are provided. Such probes and primers contain at least 8, 14, 16, 30, 100 or more contiguous nucleotides with identity to contiguous nucleotides of a CVSP16, particularly those that span nucleotides corresponding to 1978-1983 of SEQ ID No. 5. The probes and primers are optionally labelled with a detectable label, such as a radiolabel or a fluorescent tag, or can be mass differentiated for detection by mass spectrometry or other means.

Also provided are fragments thereof or oligonucleotides that can be used as probes or primers and that contain at least about 10, 14, 16 nucleotides, generally less than 1000 or less than or equal to 100, set forth in SEQ ID No. 5 (or the complement thereof), particularly those that span nt. 1978-1983; or contain at least about 30 nucleotides (or the complement thereof) or contain oligonucleotides that hybridize along their full length (or at least about 70, 80 or 90% thereof) to any such fragments or oligonucleotides. The length of the fragments are a function of the purpose for which they are used and/or the complexity of the genome of interest. Generally probes and primers contain less than about 500, 150, 100 nucleotides.

Also provided is an isolated nucleic acid molecule that includes the sequence of molecules that is complementary to the nucleotide sequence encoding CVSP16 or the portion thereof. Double-stranded RNA (dsRNA), such as RNAi also is provided.

Plasmids, Vectors and Cells

Plasmids and vectors containing the nucleic acid molecules are also provided. Cells containing the vectors, including cells that express the encoded proteins are provided. The cell can be a bacterial cell, a yeast cell, a fungal cell, a plant cell, an insect cell or an animal cell. Methods for producing a SP or single chain form of the protease domain thereof by, for example, growing the cell under conditions whereby the encoded SP is expressed by the cell, and recovering the expressed protein, are provided herein. As noted, for CVSP16, the full-length zymogens and activated proteins and activated (two- or three-chain) protease and single chain protease domains are provided.

As discussed below, the CVSP16 polypeptide, and catalytically active portions thereof, can be expressed as a secreted protein using the native signal sequence or a heterologous signal. Alternatively the protein can be expressed as inclusion bodies in the cytoplasm and isolated therefrom. The resulting protein can be treated to refold (see, e.g., EXAMPLE 1). Active protease domain can be produced by expression in inclusion bodies, isolation therefrom and denaturation followed by refolding.

C. Tumor Specificity and Tissue Expression Profiles

Each SP has a characteristic tissue expression profile; the SPs in particular, although not exclusively expressed or activated in tumors, exhibit characteristic tumor tissue expression or activation profiles. In some instances, SPs can have different activity in a tumor cell from a non-tumor cell by virtue of a change in a substrate or cofactor therefor or other factor that would alter the apparent functional activity of the SP. Hence each can serve as a diagnostic marker for particular tumors, by virtue of a level of activity and/or expression or function in a subject (i.e. a mammal, particularly a human) with neoplastic disease, compared to a subject or subjects that do not have the neoplastic disease. In addition, detection of activity (and/or expression) in a particular tissue can be indicative of neoplastic disease.

Circulating CSVP16s in body fluids can be indicative of neoplastic disease. Secreted CVSP16 or activated CVSP16 can be indicative of neoplastic disease. Also, by virtue of an activity and/or expression profile of each, CVP16s can serve as therapeutic targets, such as by administration of modulators of an activity thereof, or, as by administration of a prodrug specifically activated by one of the CVSP16s.

### Tissue Expression Profiles

### CVSP16

Results indicate that the CVSP16 transcript is strongly expressed in several tissues including kidney, stomach, colon, spleen, thyroid gland, trachea and pituitary gland. The CVSP16 transcript also is found in many other tissues at a lower level. Among tumor cell lines, the CVSP16 transcript occurs, for example, in cervical Hela S3, lung A549, leukemia K-562, Burkitt's lymphoma Raji, leukemia HL-60, colorectal SW480, Burkitt's lymphoma Daudi and leukemia MOLT-4. The CVSP16 transcript also was detected (in decreasing signal intensity) in normal breast, normal prostate, breast carcinoma cell line DU4475, prostate carcinoma cell line PC-3, prostate carcinoma cell line LNCaP, breast carcinoma cell line MDA-MB-231, and breast carcinoma cell line MDA-MB-453. Tissue expression profiles are described in Example 1.

D. Identification and Isolation of SP Protein Genes

The SP polypeptides, including CVSP16 polypeptides, or domains thereof, can be obtained by methods well known in the art for protein purification and recombinant protein expression. Any method known to those of skill in the art for identification of nucleic acids that encode desired genes can be used. Any method available in the art can be used to obtain a full length (i.e., encompassing the entire coding region) cDNA or genomic DNA clone encoding a SP protein. In particular, the polymerase chain reaction (PCR) can be used to amplify a sequence identified as being differentially expressed or encoding proteins activated at different levels in tumor and non-tumor cells or tissues, e.g., nucleic acids encoding a CVSP16 polypeptide (SEQ. NOs: 5, 6, 12 and 13), in a genomic or cDNA library. Oligonucleotide primers that hybridize to sequences at the 3′ and 5′ termini of the identified sequences can be used as primers to amplify by PCR sequences from a nucleic acid sample (RNA or DNA), typically a cDNA library, from an appropriate source (e.g., tumor or cancer tissue).

Amplification, such as PCR, can be carried out by a thermal cycler and thermostable DNA polymerase. The nucleic acid that is amplified can include mRNA or cDNA or genomic DNA from any eukaryotic species. One can choose to synthesize several different degenerate primers, for use in the PCR reactions. It also is possible to vary the stringency of hybridization conditions used in priming the PCR reactions, to amplify nucleic acid orthologs or homologs (e.g., to obtain SP protein sequences from species other than humans or to obtain human sequences with homology to CVSP16 polypeptide) by allowing for greater or lesser degrees of nucleotide sequence similarity between the known nucleotide sequence and the nucleic acid homolog being isolated. For cross species hybridization, low or moderate stringency conditions are used. For same species hybridization, moderate or high stringency conditions generally are used. After successful amplification of the nucleic acid containing all or a portion of the identified CVSP16 protein sequence or of a nucleic acid encoding all or a portion of a CVSP16 protein homolog, that segment can be molecularly cloned and sequenced, and used as a probe to isolate a complete cDNA or genomic clone. This, in turn, permits the determination of the gene's complete nucleotide sequence, the analysis of its expression, and the production of its protein product for functional analysis. Once the nucleotide sequence is determined, an open reading frame encoding the SP protein gene protein product can be determined by any method well known in the art for determining open reading frames, for example, using publicly available computer programs for nucleotide sequence analysis. Once an open reading frame is defined, it is routine to determine the amino acid sequence of the protein encoded by the open reading frame. In this way, the nucleotide sequences of the entire SP protein genes as well as the amino acid sequences of SP proteins and analogs can be identified.

Any eukaryotic cell potentially can serve as the nucleic acid source for the molecular cloning of the SP protein gene. The nucleic acids can be isolated from vertebrate, mammalian, human, porcine, bovine, feline, avian, equine, canine, as well as additional primate sources, insects, plants, and other sources. The DNA can be obtained by standard procedures known in the art from cloned DNA (e.g., a DNA “library”), by chemical synthesis, by cDNA cloning, or by the cloning of genomic DNA, or fragments thereof, purified from the desired cell (see, e.g., Sambrook et al. (2001) Molecular Cloning: A Laboratory Manual (3rd Ed.), Vol. 1-3, Cold Spring Harbor Laboratory, Cold Spring Harbor, N.Y.). Clones derived from genomic DNA can contain regulatory and intron DNA regions in addition to coding regions; clones derived from cDNA contains only exon sequences. Whatever the source, the gene should be molecularly cloned into a suitable vector for propagation of the gene.

In the molecular cloning of the gene from genomic DNA, DNA fragments are generated, some of which encode the desired gene. The DNA can be cleaved at specific sites using various restriction enzymes. Alternatively, one can use DNAse in the presence of manganese to fragment the DNA, or the DNA can be physically sheared, for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired gene can be accomplished in a number of ways. For example, a portion of the SP protein (of any species) gene (e.g., a PCR amplification product obtained as described above or an oligonucleotide having a sequence of a portion of the known nucleotide sequence) or its specific RNA, or a fragment thereof can be purified and labeled, and the generated DNA fragments can be screened by nucleic acid hybridization to the labeled probe (Benton and Davis, Science 196:180 (1977); Grunstein and Hogness, Proc. Natl. Acad. Sci. U.S.A. 72:3961 (1975)). Those DNA fragments with substantial homology to the probe hybridize. It also is possible to identify the appropriate fragment by restriction enzyme digestion(s) and comparison of fragment sizes with those expected according to a known restriction map if such is available or by DNA sequence analysis and comparison to the known nucleotide sequence of SP protein. Further selection can be carried out on the basis of the properties of the gene. Alternatively, the presence of the gene can be detected by assays based on the physical, chemical, or immunological properties of its expressed product. For example, cDNA clones, or DNA clones which hybrid-select the proper mRNA, can be selected which produce a protein that, e.g., has similar or identical electrophoretic migration, isoelectric focusing behavior, proteolytic digestion maps, antigenic properties, serine protease activity. If an anti-SP protein antibody is available, the protein can be identified by binding of labeled antibody to the putatively SP protein synthesizing clones, in an ELISA (enzyme-linked immunosorbent assay)-type procedure.

Alternatives to isolating the CVSP16 polypeptide genomic DNA include, but are not limited to, chemically synthesizing the gene sequence from a known sequence or making cDNA to the mRNA that encodes the SP protein. For example, RNA for cDNA cloning of the SP protein gene can be isolated from cells expressing the protein. The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pBR322 or pUC plasmid derivatives or the Bluescript vector (Stratagene, La Jolla, Calif.). The insertion into a cloning vector can, for example, be accomplished by ligating the DNA fragment into a cloning vector which has complementary cohesive termini. Insertion can be effected using TOPO cloning vectors (INVITROGEN, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and SP protein gene can be modified by homopolymeric tailing. Recombinant molecules can be introduced into host cells via, for example, transformation, transfection, infection, electroporation and sonorporation, so that many copies of the gene sequence are generated.

In specific embodiments, transformation of host cells with recombinant DNA molecules that incorporate the isolated SP protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

E. Vectors, Plasmids and Cells that Contain Nucleic Acids Encoding a SP Protein or Protease Domain Thereof and Expression of SP Proteins

Vectors and Cells

For recombinant expression of one or more of the SP proteins, the nucleic acid containing all or a portion of the nucleotide sequence encoding the SP protein can be inserted into an appropriate expression vector, i.e., a vector that contains the necessary elements for the transcription and translation of the inserted protein coding sequence. The necessary transcriptional and translational signals also can be supplied by the native promoter for SP genes, and/or their flanking regions.

Also provided are vectors that contain nucleic acid encoding the SPs. Cells containing the vectors are also provided. The cells include eukaryotic and prokaryotic cells, and the vectors are any suitable for use therein.

Prokaryotic and eukaryotic cells, including endothelial cells, containing the vectors are provided. Such cells include bacterial cells, yeast cells, fungal cells, Archea, plant cells, insect cells and animal cells. The cells are used to produce a SP protein or protease domain thereof by growing the above-described cells under conditions whereby the encoded SP protein or protease domain of the SP protein is expressed by the cell, and recovering the expressed protease domain protein. For purposes herein, the protease domain can be secreted into the medium.

In one embodiment, the vectors include a sequence of nucleotides that encodes a polypeptide that has protease activity and contains all or a portion of the protease domain, or multiple copies thereof, of a SP protein are provided. Also provided are vectors that contain a sequence of nucleotides that encodes the protease domain and additional portions of a SP protein up to and including a full length SP protein, as well as multiple copies thereof, are also provided. The vectors can be selected for expression of the SP protein or protease domain thereof in the cell or such that the SP protein is expressed as a secreted protein. When the protease domain is expressed the nucleic acid is linked to nucleic acid encoding a secretion signal, such as the Saccharomyces cerevisiae a mating factor signal sequence or a portion thereof, or the native-signal sequence.

A variety of host-vector systems can be used to express the protein coding sequence. These include but are not limited to mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding SP protein, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for SP protein. Promoters which can be used include but are not limited to the SV40 early promoter (Bernoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Villa-Kamaroff et al., Proc. Natl. Acad. Sci. USA 75:3727-3731 1978)) or the tac promoter (DeBoer et al., Proc. Natl. Acad. Sci. USA 80:21-25 (1983)); see also “Useful Proteins from Recombinant Bacteria”: in Scientific American 242:79-94 (1980)); plant expression vectors containing the nopaline synthetase promoter (Herrar-Estrella et al., Nature 303:209-213 (1984)) or the cauliflower mosaic virus 35S RNA promoter (Garder et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in myeloid cells (Mogram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Sani, Nature 314:283-286 (1985)), and gonadotrophic releasing hormone gene control region which is active in gonadotrophs of the hypothalamus (Mason et al., Science 234:1372-1378 (1986)).

In a specific embodiment, a vector is used that contains a promoter operably linked to nucleic acids encoding a SP protein, or a domain, fragment, derivative or homolog, thereof, one or more origins of replication, and optionally, one or more selectable markers (e.g., an antibiotic resistance gene). Expression vectors containing the coding sequences, or portions thereof, of a SP protein, are made, for example, by subcloning the coding portions into the EcoRI restriction site of each of the three pGEX vectors (glutathione S-transferase expression vectors (Smith and Johnson, Gene 7:31-40 (1988)). This allows for the expression of products in the correct reading frame. Vectors and systems for expression of the protease domains of the SP proteins include the well known Pichia vectors (available, for example, from Invitrogen, San Diego, Calif.), particularly those designed for secretion of the encoded proteins. One exemplary vector is described in the EXAMPLES.

Plasmids for transformation of E. coli cells, include, for example, the pET expression vectors (see, U.S. Pat. No. 4,952,496; available from NOVAGEN, Madison, Wis.; see, also literature published by Novagen describing the system). Such plasmids include pET 11a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b and pET19b (NOVAGEN, Madison, Wis.), which contain a His-Tag™ leader sequence for use in purification with a His column and a thrombin cleavage site that permits cleavage following purification over the column, the T7-lac promoter region and the T7 terminator.

The vectors are introduced into host cells, such as Pichia cells and bacterial cells, such as E. coli, and the proteins expressed therein. Pichia strains, which are known and readily available, include, for example, GS115. Bacterial hosts can contain chromosomal copies of DNA encoding T7 RNA polymerase operably linked to an inducible promoter, such as the lacUV promoter (see, U.S. Pat. No. 4,952,496). Such hosts include, but are not limited to, the lysogenic E. coli strain BL21 (DE3).

Expression and Production of Proteins

The CVSP16 domains, derivatives and analogs can be produced by various methods known in the art. For example, once a recombinant cell expressing a SP protein, or a domain, fragment or derivative thereof, is identified, the individual gene product can be isolated and analyzed. This is achieved by assays based on the physical and/or functional properties of the protein, including, but not limited to, radioactive labeling of the product followed by analysis by gel electrophoresis, immunoassay, cross-linking to marker-labeled product.

The CVSP16 polypeptides can be isolated and purified by standard methods known in the art (either from natural sources or recombinant host cells expressing the complexes or proteins), including but not restricted to column chromatography (e.g., ion exchange, affinity, gel exclusion, reversed-phase high pressure and fast protein liquid), differential centrifugation, differential solubility, or by any other standard technique used for the purification of proteins. Functional properties can be evaluated using any suitable assay known in the art.

Once a SP protein or its domain or derivative is identified, the amino acid sequence of the protein can be deduced from the nucleotide sequence of the gene which encodes it. In addition, domains, analogs and derivatives of a SP protein can be chemically synthesized by standard chemical methods known in the art (e.g. see Hunkapiller et al. (1984) Nature 310:105-111). For example, a peptide corresponding to a portion of a SP protein, which includes the desired domain or which mediates the desired activity in vitro can be synthesized by use of a peptide synthesizer. Furthermore, if desired, nonclassical amino acids or chemical amino acid analogs can be introduced as a substitution or addition into the SP protein sequence. Non-classical amino acids include but are not limited to the D-isomers of the common amino acids, a-amino isobutyric acid, 4-aminobutyric acid, Abu, 2-aminobutyric acid, ε-Abu, e-Ahx, 6-amino hexanoic acid, Aib, 2-amino isobutyric acid, 3-amino propionoic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Ca-methyl amino acids, Na-methyl amino acids, and amino acid analogs in general. The amino acids can be D (dextrorotatory) or L (levorotatory).

Manipulations of SP protein sequences can be made at the protein level. Also contemplated herein are SP proteins, domains thereof, derivatives or analogs or fragments thereof, which are differentially modified during or after translation, e.g., by glycosylation, acetylation, phosphorylation, amidation, derivatization by known protecting/blocking groups, proteolytic cleavage, linkage to an antibody molecule or other cellular ligand. Any of numerous chemical modifications can be carried out by known techniques, including but not limited to specific chemical cleavage by cyanogen bromide, trypsin, chymotrypsin, papain, V8 protease, NaBH₄, acetylation, formylation, oxidation, reduction and metabolic synthesis in the presence of tunicamycin.

In cases where natural products are suspected of having a mutation or are isolated from new species, the amino acid sequence of the SP protein isolated from the natural source, as well as those expressed in vitro, or from synthesized expression vectors in vivo or in vitro, can be determined from analysis of the DNA sequence, or alternatively, by direct sequencing of the isolated protein. Such analysis can be performed by manual sequencing or through use of an automated amino acid sequenator.

In particular, the protease domain (or full length or other fragment) of the CVSP16 can be expressed intracellularly without a signal sequence, which results in accumulation or formation of inclusion bodies containing the protease domain. The inclusion bodies are isolated, denatured, solubilized and refolded to yield a protease domain (or full length or other fragment), which is then activated by cleavage at the activation cleavage site.

Modifications

A variety of modifications of the SP proteins and domains are contemplated herein. A SP-encoding nucleic acid molecule can be modified by any of numerous strategies known in the art (Sambrook et al. (1989) Molecular Cloning, A Laboratory Manual, 2d ed., Cold Spring Harbor Laboratory, Cold Spring Harbor, N.Y., and Sambrook et al. (2001) Molecular Cloning: A Laboratory Manual (3rd Ed.), Vol. 1-3, Cold Spring Harbor Laboratory, Cold Spring Harbor, N.Y.). The sequences can be cleaved at appropriate sites with restriction endonuclease(s), followed by further enzymatic modification if desired, isolated, and ligated in vitro. In the production of the gene encoding a domain, derivative or analog of SP, care should be taken to ensure that the modified gene retains the original translational reading frame, uninterrupted by translational stop signals, in the gene region where the desired activity is encoded.

Additionally, the SP-encoding nucleic acid molecules can be mutated in vitro or in vivo, to create and/or destroy translation, initiation, and/or termination sequences, or to create variations in coding regions and/or form new restriction endonuclease sites or destroy pre-existing ones, to facilitate further in vitro modification. Also, as described herein muteins with primary sequence alterations, such as replacements of Cys residues and elimination of glycosylation sites are contemplated. Such mutations can be effected by any technique for mutagenesis known in the art, including, but not limited to, chemical mutagenesis and in vitro site-directed mutagenesis (Hutchinson et al., J. Biol. Chem. 253:6551-6558 (1978)), use of TAB® linkers (Pharmacia). In one embodiment, for example, a SP protein or domain thereof is modified to include a fluorescent label. In other specific embodiments, the SP protein is modified to have a heterofunctional reagent, which can be used to crosslink the members of the complex.

F. Screening Methods

The single chain protease domains can be used in a variety of methods to identify compounds that modulate the activity thereof. For SPs that exhibit higher activity or expression in tumor cells, compounds that inhibit the proteolytic activity are of particular interest. For any SPs that are active at lower levels in tumor cells, compounds or agents that enhance the activity are potentially of interest. In all instances the identified compounds include agents that are candidate cancer treatments.

Several types of assays are described herein. It is understood that the protease domains can be used in other assays. A single-chain protease domain can exhibit catalytic activity. As such the protease domains are useful for in vitro screening assays, including, for example in binding assays.

The CVSP16 full length zymogens, activated enzymes, single-, two- and three-chain protease domains are contemplated for use in any screening assay known to those of skill in the art, including those provided herein. Hence the following description, if directed to proteolytic assays is intended to apply to use of an active single-chain protease domain or a catalytically active portion thereof. Other assays, such as binding assays are provided herein, particularly for use with a CVSP16, including any variants, such as splice variants thereof.

1. Catalytic Assays for Identification of Agents that Modulate the Protease Activity of a SP Protein

Methods for identifying a modulator of the catalytic activity of a SP, particularly a single chain protease domain or catalytically active portion thereof, are provided herein. The methods can be practiced by: a) contacting the CVSP16, a full-length zymogen or activated form, and particularly a single-chain domain thereof, with a substrate of the CVSP16 in the presence of a test substance, and detecting the proteolysis of the substrate, whereby the activity of the CVSP16 is assessed, and comparing the activity to a control. For example, the control can be the activity of the CVSP16 assessed by contacting a CVSP16, including a full-length zymogen or activated form, and particularly a single-chain domain thereof, with a substrate of the CVSP16, and detecting the proteolysis of the substrate, whereby the activity of the CVSP16 is assessed. The results in the presence and absence of the test compounds are compared. A difference in the activity indicates that the test substance modulates the activity of the CVSP16. Modulators, such as activators, of CVSP16 activity or activation also are contemplated; such assays are discussed below.

In one embodiment a plurality of the test substances are screened simultaneously in the above screening method. In another embodiment, the CVSP16 is isolated from a target cell as a means for then identifying agents that are potentially specific for the target cell.

In another embodiment, a test substance is potentially a therapeutic compound. A difference in CVSP16 activity is measured in the presence and in the absence of the test substance. A difference indicates that the target cell responds to the compound.

One method includes the steps of (a) contacting the CVSP16 polypeptide or protease domain thereof with one or a plurality of test compounds under conditions conducive to interaction between the ligand and the compounds; and (b) identifying one or more compounds in the plurality that specifically binds to the ligand.

Another method provided herein includes the steps of a) contacting a CVSP16 polypeptide or protease domain thereof with a substrate of the CVSP16 polypeptide, and detecting the proteolysis of the substrate, whereby the activity of the CVSP16 polypeptide is assessed; b) contacting the CVSP16 polypeptide with a substrate of the CVSP16 polypeptide in the presence of a test substance, and detecting the proteolysis of the substrate, whereby the activity of the CVSP16 polypeptide is assessed; and c) comparing the activity of the CVSP16 polypeptide assessed in steps a) and b), whereby the activity measured in step a) differs from the activity measured in step b) indicates that the test substance modulates the activity of the CVSP16 polypeptide.

In another embodiment, a plurality of the test substances are screened simultaneously. In comparing the activity of a CVSP16 polypeptide in the presence and absence of a test substance to assess whether the test substance is a modulator of the CVSP16 polypeptide, it is unnecessary to assay the activity in parallel, although such parallel measurement is typical. It is possible to measure the activity of the CVSP16 polypeptide at one time point and compare the measured activity to a historical value of the activity of the CVSP16 polypeptide.

For instance, one can measure the activity of the CVSP16 polypeptide in the presence of a test substance and compare with historical value of the activity of the CVSP16 polypeptide measured previously in the absence of the test substance, and vice versa. This can be accomplished, for example, by providing the activity of the CVSP16 polypeptide on an insert or pamphlet provided with a kit for conducting the assay. Methods for selecting substrates for a particular SP are described in the EXAMPLES, and particular proteolytic assays are described.

Combinations and kits containing the combinations optionally including instructions for performing the assays are provided. The combinations include a CVSP16 polypeptide and a substrate of the CVSP16-polypeptide to be assayed; and, optionally reagents for detecting proteolysis of the substrate. The substrates, which can be chromogenic or fluorogenic molecules, including proteins, subject to proteolysis by a particular CVSP16 polypeptide, can be identified empirically by testing the ability of the CVSP16 polypeptide to cleave the test substrate. Substrates that are cleaved most effectively (i.e., at the lowest concentrations and/or fastest rate or under desirable conditions), are identified.

Additionally provided herein is a kit containing the above-described combination. The kit optionally includes instructions for identifying a modulator of the activity of a CVSP16 polypeptide. Any CVSP16 polypeptide is contemplated as target for identifying modulators of the activity thereof.

2. Binding Assays

Also provided herein are methods for identification and isolation of agents, particularly compounds that bind to CVSP16s. The assays are designed to identify agents that bind to the zymogen form, the single chain isolated protease domain (or a protein, other than a CVSP16 polypeptide, that contains the protease domain of a CVSP16 polypeptide), and to the activated form, including the activated form derived from the full length zymogen or from a polypeptide that contains the protease domain. The identified compounds are candidates or leads for identification of compounds for treatments of tumors and other disorders and diseases involving aberrant proliferation and/or angiogenesis. The CVSP16 polypeptides used in the methods include any CVSP16 polypeptide as defined herein, including the CVSP16 single chain protease domain or proteolytically active portion thereof.

A variety of methods are provided herein. These methods can be performed in solution or in solid phase reactions in which the CVSP16 polypeptide(s) or protease domain(s) thereof are linked, either directly or indirectly via a linker, to a solid support. Screening assays are described in the Examples, and these assays can be used to identify candidate compounds. For purposes herein, all binding assays described above are provided for CVSP16.

Methods for identifying an agent, such as a compound, that specifically binds to a CVSP16 single chain protease domain, a zymogen or full-length activated CVSP16 and/or two-chain protease domain thereof or other polypeptides provided herein are provided. The method can be practiced by (a) contacting the CVSP16 with one or a plurality of test agents under conditions conducive to binding between the CVSP16 and an agent; and (b) identifying one or more agents within the plurality that specifically binds to one or more CVSP16 forms.

For example, in practicing such methods the CVSP16 polypeptide is mixed with a potential binding partner or an extract or fraction of a cell under conditions that allow the association of potential binding partners with the polypeptide. After mixing, peptides, polypeptides, proteins or other molecules that have become associated with a CVSP16 are separated from the mixture. The binding partner that bound to the CVSP16 can then be removed and further analyzed. To identify and isolate a binding partner, the entire protein, for instance the entire disclosed protein of SEQ ID Nos. 6 can be used. Alternatively, a fragment of the protein can be used.

A variety of methods can be used to obtain cell extracts or body fluids, such as blood, serum, urine, sweat, synovial fluid, CSF and other such fluids. For example, cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the CVSP16 under conditions in which association of the protein with the binding partner can occur. A variety of conditions can be used, including conditions that resemble conditions found in the cytoplasm of a human cell. Features such as osmolarity, pH, temperature, and the concentration of cellular extract used, can be varied to optimize the association of the protein with the binding partner. Similarly, methods for isolation of molecules of interest from body fluids are known.

After mixing under appropriate conditions, the bound complex is separated from the mixture. A variety of techniques can be used to separate the mixture. For example, antibodies specific to a CVSP16 can be used to immunoprecipitate the binding partner complex. Alternatively, standard chemical separation techniques such as chromatography and density/sediment centrifugation can be used.

After removing the non-associated cellular constituents in the extract, the binding partner can be dissociated from the complex using conventional methods. For example, dissociation can be accomplished by altering the salt concentration and/or pH of the mixture.

To aid in separating associated binding partner pairs from the mixed extract, the CVSP16 can be immobilized on a solid support. For example, the protein can be attached to a nitrocellulose matrix or acrylic beads. Attachment of the protein or a fragment thereof to a solid support aids in separating peptide/binding partner pairs from other constituents found in the extract. The identified binding partners can be either a single protein or a complex made up of two or more proteins.

Alternatively, the nucleic acid molecules encoding the single chain proteases can be used in a yeast two-hybrid system. The yeast two-hybrid system has been used to identify other protein partner pairs and can readily be adapted to employ the nucleic acid molecules herein described.

Another in vitro binding assay, particularly for a CVSP16, uses a mixture of a polypeptide that contains at least the catalytic domain of one of these proteins and one or more candidate binding targets or substrates. After incubating the mixture under appropriate conditions, the ability of the CVSP16 or a polypeptide fragment thereof containing the catalytic domain to bind to or interact with the candidate substrate is assessed. For cell-free binding assays, one of the components includes or is coupled to a detectable label. The label can provide for direct detection, such as radioactivity, luminescence, including fluorescence, optical or electron density, or indirect detection such as an epitope tag and an enzyme. A variety of methods can be employed to detect the label depending on the nature of the label and other assay components. For example, the label can be detected bound to the solid substrate or a portion of the bound complex containing the label can be separated from the solid substrate, and the label thereafter detected.

3. Detection of Signal Transduction

Secreted CVSPs, such as CVSP16, can be involved in signal transduction either directly by binding to or interacting with a cell surface receptor or indirectly by activating proteins, such as pro-growth factors that can initiate signal transduction. Assays for assessing signal transduction are well known to those of skill in the art, and can be adapted for use with the CVSP16 polypeptide.

Assays for identifying agents that affect or alter signal transduction mediated directly or indirectly, such as via activation of a pro-growth factor, by a CVSP16, particularly the full length or a portion sufficient to exhibit proteolytic or binding activity. Such assays, include, for example, transcription based assays in which modulation of a transduced signal is assessed by detecting an effect on an expression from a reporter gene (see, e.g., U.S. Pat. No. 5,436,128).

4. Methods for Identifying Agents that Modulate the Expression a Nucleic Acid Encoding a CVSP16

Another embodiment provides methods for identifying agents that modulate the expression of a nucleic acid encoding a CVSP16. Such assays use any available means of monitoring for changes in the expression level of the nucleic acids encoding a CVSP16.

In one assay format, cell lines that contain reporter gene fusions between the open reading frame of CVSP16 or a domain thereof, particularly the protease domain and any assayable fusion partner can be prepared. Numerous assayable fusion partners are known and readily available including the firefly luciferase gene and the gene encoding chloramphenicol acetyltransferase (Alam et al., Anal. Biochem. 188: 245-54 (1990)). Cell lines containing the reporter gene fusions are then exposed to the agent to be tested under appropriate conditions and time. Differential expression of the reporter gene between samples exposed to the agent and control samples identifies agents which modulate the expression of a nucleic acid encoding a CVSP16.

Additional assay formats can be used to monitor the ability of the agent to modulate the expression of a nucleic acid encoding a CVSP16. For instance, mRNA expression can be monitored directly by hybridization to the nucleic acids. Cell lines are exposed to the agent to be tested under appropriate conditions and time and total RNA or mRNA is isolated by standard procedures (see, e.g., Sambrook et al. (2001) Molecular Cloning: A Laboratory Manual (3rd Ed.), Vol. 1-3, Cold Spring Harbor Laboratory, Cold Spring Harbor, N.Y.). Probes to detect differences in RNA expression levels between cells exposed to the agent and control cells can be prepared from the nucleic acids. It is typical, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes can be designed from the nucleic acids through methods known in the art. For instance, the G+C content of the probe and the probe length can affect probe binding to its target sequence. Methods to optimize probe specificity are commonly available (see, e.g., Sambrook et al. (2001) Molecular Cloning: A Laboratory Manual (3rd Ed.), Vol. 1-3, Cold Spring Harbor Laboratory, Cold Spring Harbor, N.Y.; Sambrook et al. (1989) MOLECULAR CLONING: A LABORATORY MANUAL, 2nd Ed. Cold Spring Harbor Laboratory Press); and Ausubel et al. (1995) CURRENT PROTOCOLS IN MOLECULAR BIOLOGY, Greene Publishing Co., NY).

Hybridization conditions are modified using known methods ((see, e.g., Sambrook et al. (2001) Molecular Cloning: A Laboratory Manual (3rd Ed.), Vol. 1-3, Cold Spring Harbor Laboratory, Cold Spring Harbor, N.Y.; Sambrook et al. (1989) MOLECULAR CLONING: A LABORATORY MANUAL, 2nd Ed. Cold Spring Harbor Laboratory Press); and Ausubel et al. (1995) CURRENT PROTOCOLS IN MOLECULAR BIOLOGY, Greene Publishing Co., NY), as required for each probe. Hybridization of total cellular RNA or RNA enriched for polyA RNA can be accomplished in any available format. For instance, total cellular RNA or RNA enriched for polyA RNA can be affixed to a solid support, and the solid support exposed to at least one probe comprising at least one, or part of one of the nucleic acid molecules under conditions in which the probe specifically hybridizes. Alternatively, nucleic acid fragments comprising at least one, or part of one of the sequences can be affixed to a solid support, such as a porous glass wafer. The glass wafer can then be exposed to total cellular RNA or polyA RNA from a sample under conditions in which the affixed sequences specifically hybridize. Such glass wafers and hybridization methods are widely available, for example, those disclosed by Beattie (WO 95/11755). By examining for the ability of a given probe to specifically hybridize to an RNA sample from an untreated cell population and from a cell population exposed to the agent, agents which up or down regulate the expression of a nucleic acid encoding the CVSP16 polypeptide, are identified.

In one format, the relative amounts of a protein between a cell population that has been exposed to the agent to be tested compared to an un-exposed control cell population can be assayed (e.g., a prostate cancer cell line, a lung cancer cell line, a colon cancer cell line or a breast cancer cell line). In this format, probes, such as specific antibodies, are used to monitor the differential expression or level of activity of the protein in the different cell populations or body fluids. Cell lines or populations or body fluids are exposed to the agent to be tested under appropriate conditions and time. Cellular lysates or body fluids can be prepared from the exposed cell line or population and from a control, unexposed cell line or population or unexposed body fluid. The cellular lysates or body fluids are then analyzed with the probe.

For example, N- and C-terminal fragments of the CVSP16 can be expressed in bacteria and used to search for proteins which bind to these fragments. Fusion proteins, such as His-tag or GST fusion to the N- or C-terminal regions of the CVSP16 can be prepared for use as a binding partner. These fusion proteins can be coupled to, for example, Glutathione-Sepharose beads and then probed with cell lysates or body fluids. Prior to lysis, the cells or body fluids can be treated with a candidate agent which can modulate a CVSP16 or proteins that interact with domains thereon. Lysate proteins binding to the fusion proteins can be resolved by SDS-PAGE, isolated and identified by protein sequencing or mass spectroscopy, as is known in the art.

Antibody probes are prepared by immunizing suitable mammalian hosts in appropriate immunization protocols using the peptides, polypeptides or proteins if they are of sufficient length (e.g., 4, 5, 6, 7, 8, 9, 10, 11, 12, 13, 14, 15, 20, 25, 30, 35, 40 or more consecutive amino acids of the CVSP16 polypeptide or if required to enhance immunogenicity, conjugated to suitable carriers. Methods for preparing immunogenic conjugates with carriers, such as bovine serum albumin (BSA), keyhole limpet hemocyanin (KLH), or other carrier proteins are well known in the art. In some circumstances, direct conjugation using, for example, carbodiimide reagents can be effective; in other instances linking reagents such as those supplied by Pierce Chemical Co., Rockford, Ill., can be desirable to provide accessibility to the hapten. Hapten peptides can be extended at either the amino or carboxy terminus with a Cys residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

Anti-peptide antibodies can be generated using synthetic peptides corresponding to, for example, the carboxy terminal amino acids of the CVSP16. Synthetic peptides can be as small as 1-3 amino acids in length, generally at least 4 or more amino acid residues long. The peptides can be coupled to KLH using standard methods and can be immunized into animals, such as rabbits or ungulate. Polyclonal antibodies then can then be purified, for example using Actigel beads containing the covalently bound peptide, or other reagents for affinity purification methods or by purifying all of the IgGs, using Protein A or Protein G columns or other such methods.

While the polyclonal antisera produced in this way can be satisfactory for some applications. For pharmaceutical compositions monoclonal preparations are generally used. Immortalized cell lines which secrete the desired monoclonal antibodies can be prepared using the standard method of Kohler et al., (Nature 256: 495-7 (1975)) or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in vivo via ascites fluid. Of particular interest, are monoclonal antibodies that recognize the catalytic domain of a CVSP16.

Additionally, the zymogen, a single chain or a three-chain or a two-chain form of the CVSP16 can be used to make monoclonal antibodies that recognize conformational epitopes. The desired monoclonal antibodies are then recovered from the culture supernatant or from the ascites supernatant. Fragments of the monoclonals or the polyclonal antisera that contain the antigen binding portion can be used as antagonists, as well as the intact antibodies. Immunologically reactive fragments, such as the Fab, Fab′, or F(ab′)₂ fragments are often used, especially in a therapeutic context, as these fragments are generally less immunogenic than the whole immunoglobulin. Regions that bind specifically to the desired regions of receptor also can be produced in the context of chimeras with multiple species origin. Fully human antibodies also can be prepared using, for example, either transgenic mice that contain human immunoglobulin genes or phage display libraries of human antibodies.

G. Assay Formats and Selection of Test Substances that Modulate at Least One Activity of a CVSP16 Polypeptide

Methods for identifying agents that modulate at least one activity of a CVSP16 are provided. Methods include phage display and include other methods for assessing alterations in the activity of an CVSP16. Such methods or assays can use any means of monitoring or detecting the desired activity. A variety of formats and detection protocols are known for performing screening assays. Any such formats and protocols can be adapted for identifying modulators of CVSP16 polypeptide activities. The following includes a discussion of exemplary protocols.

1. High Throughput Screening Assays

Although the above-described assay can be conducted where a single CVSP16 polypeptide is screened, and/or a single test substance is screened in one assay, the assay typically is conducted in a high throughput screening mode, i.e., a plurality of the SP proteins are screened against and/or a plurality of the test substances are screened simultaneously (See generally, High Throughput Screening: The Discovery of Bioactive Substances (Devlin, Ed.) Marcel Dekker, 1997; Sittampalam et al., Curr. Opin. Chem. Biol., 1:384-91 (1997); and Silverman et al., Curr. Opin. Chem. Biol., 2:397-403 (1998)). For example, the assay can be conducted in a multi-well (e.g., 24-, 48-, 96-, 384-, 1536-well or higher density), chip or array format.

High-throughput screening (HTS) is the process of testing a large number of diverse chemical structures against disease targets to identify “hits” (Sittampalam et al., Curr. Opin. Chem. Biol., 1:384-91 (1997)). Current state-of-the-art HTS operations are highly automated and computerized to handle sample preparation, assay procedures and the subsequent processing of large volumes of data.

Detection technologies employed in high-throughput screens depend on the type of biochemical pathway being investigated (Sittampalam et al., Curr. Opin. Chem. Biol., 1:384-91 (1997)). These methods include, radiochemical methods, such as the scintillation proximity assays (SPA), which can be adapted to a variety of enzyme assays (Lerner et al., J. Biomol. Screening, 1:135-143 (1996); Baker et al., Anal. Biochem., 239:20-24 (1996); Baum et al., Anal. Biochem., 237:129-134 (1996); and Sullivan et al., J. Biomol. Screening 2:19-23 (1997)) and protein-protein interaction assays (Braunwalder et al., J. Biomol. Screening 1:23-26 (1996); Sonatore et al., Anal. Biochem. 240:289-297 (1996); and Chen et al., J. Biol. Chem. 271:25308-25315 (1996)), and non-isotopic detection methods, including but are not limited to, calorimetric and luminescence detection methods, resonance energy transfer (RET) methods, time-resolved fluorescence (HTRF) methods, cell-based fluorescence assays, such as fluorescence resonance energy transfer (FRET) procedures (see, e.g., Gonzalez et al., Biophys. J., 69:1272-1280 (1995)), fluorescence polarization or anisotropy methods (see, e.g., Jameson et al., Methods Enzymol. 246:283-300 (1995); Jolley, J. Biomol. Screening 1:33-38 (1996); Lynch et al., Anal. Biochem. 247:77-82 (1997)), fluorescence correlation spectroscopy (FCS) and other such methods.

2. Test Substances

Test compounds, including small molecules, antibodies, proteins, nucleic acids, peptides, natural products, mixtures of natural products, derivatives (e.g, chemical derivatives) of natural products, and libraries and collections thereof, can be screened in the above-described assays and assays described below to identify compounds that modulate the activity of a CVSP16 polypeptide. Rational drug design methodologies that rely on computational chemistry can be used to screen and identify candidate compounds.

Test compounds (agents) that are assayed in the methods can be produced and obtained by any method known to those of skill in the art. For example, they can be randomly selected or rationally selected or designed. The agents can be, as examples, peptides, small molecules, and carbohydrates. A skilled artisan can readily recognize that there is no limit to the structural nature of the agents. The peptide agents can be prepared using standard solid phase (or solution phase) peptide synthesis methods, as is known in the art. In addition, the DNA encoding these peptides can be synthesized using commercially available oligonucleotide synthesis instrumentation and produced recombinantly using standard recombinant production systems. The production using solid phase peptide synthesis is necessitated if non-gene-encoded amino acids are to be included.

The compounds identified by the screening methods include inhibitors, including antagonists, and agonists. Compounds for screening include any compounds and collections of compounds available, known or that can be prepared.

### a. Selection of Compounds

Compounds can be selected for their potency and selectivity of inhibition of serine proteases, especially a CVSP16 polypeptide. As described herein, and as generally known, a target serine protease and its substrate are combined under assay conditions permitting reaction of the protease with its substrate. The assay is performed in the absence of test compound, and in the presence of increasing concentrations of the test compound. The concentration of test compound at which 50% of the serine protease activity is inhibited by the test compound is the IC₅₀ value (Inhibitory Concentration) or EC₅₀ (Effective Concentration) value for that compound. Within a series or group of test compounds, those having lower IC₅₀ or EC₅₀ values are considered more potent inhibitors of the serine protease than those compounds having higher IC₅₀ or EC₅₀ values. The IC₅₀ measurement is often used for more simplistic assays, whereas the EC₅₀ is often used for more complicated assays, such as those employing cells.

Typically candidate compounds have an IC₅₀ value of 100 nM or less as measured in an in vitro assay for inhibition of CVSP16 polypeptide activity. The test compounds also are evaluated for selectivity toward a serine protease. As described herein, and as generally known, a test compound is assayed for its potency toward a panel of serine proteases and other enzymes and an IC₅₀ value or EC₅₀ value is determined for each test compound in each assay system. A compound that demonstrates a low IC₅₀ value or EC₅₀ value for the target enzyme, e.g., CVSP16 polypeptide, and a higher IC₅₀ value or EC₅₀ value for other enzymes within the test panel (e.g., urokinase tissue plasminogen activator, thrombin, Factor Xa), is considered to be selective toward the target enzyme. Generally, a compound is deemed selective if its IC₅₀ value or EC₅₀ value in the target enzyme assay is at least 2-fold, 5-fold, 10-fold (or higher-fold) less than the next smallest IC₅₀ value or EC₅₀ value measured in the selectivity panel of enzymes.

Compounds are also evaluated for their activity in vivo. The type of assay chosen for evaluation of test compounds depends on the pathological condition to be treated or prevented by use of the compound, as well as the route of administration to be evaluated for the test compound.

For instance, to evaluate the activity of a compound to reduce tumor growth through inhibition of CVSP16 polypeptide, the procedures described by Jankun et al., Canc. Res. 57:559-563 (1997). Briefly, the ATCC cell lines DU145 and LnCaP are injected into SCID mice. After tumors are established, the mice are given test compound according to a dosing regime determined from the compound's in vitro characteristics. The Jankun et al. compound was administered in water. Tumor volume measurements are taken twice a week for about five weeks. A compound is deemed active if an animal to which the compound was administered exhibited decreased tumor volume, as compared to animals receiving appropriate control compounds.

Another in vivo experimental model designed to evaluate the effect of p-aminobenzamidine, a swine protease inhibitor, on reducing tumor volume is described by Billström et al., Int. J. Cancer 61:542-547 (1995).

To evaluate the ability of a compound to reduce the occurrence of, or inhibit, metastasis, the procedures described by Kobayashi et al. Int. J. Canc. 57:727-733d (1994) can be employed. Briefly, a murine xenograft selected for high lung colonization potential is injected into C57B1/6 mice i.v. (experimental metastasis) or s.c. into the abdominal wall (spontaneous metastasis). Various concentrations of the compound to be tested can be admixed with the tumor cells in Matrigel prior to subcutaneous injection. Daily i.p. injections of the test compound are made either on days 1-6 or days 7-13 after tumor inoculation. The animals are sacrificed about three or four weeks after tumor inoculation, and the lung tumor colonies are counted. Evaluation of the resulting data permits a determination as to efficacy of the test compound, optimal dosing and route of administration.

The activity of the tested compounds toward decreasing tumor volume and metastasis can be evaluated in a model, such as that described in Rabbani et al., Int. J. Cancer 63:840-845 (1995) to evaluate its inhibitor. There, Mat LyLu tumor cells were injected into the flank of Copenhagen rats. The animals were implanted with osmotic minipumps to continuously administer various doses of test compound for up to three weeks. The tumor mass and volume of experimental and control animals were evaluated during the experiment, as were metastatic growths. Evaluation of the resulting data permits a determination as to efficacy of the test compound, optimal dosing, and route of administration. Some of these authors described a related protocol in Xing et al., Canc. Res. 57:3585-3593 (1997).

To evaluate the anti-angiogenesis activity of a compound, a rabbit cornea neovascularization model can be employed (see, e.g., Avery et al. (1990) Arch. Ophthalmol. 108:1474-147). Avery et al. describes anesthetizing New Zealand albino rabbits and then making a central corneal incision and forming a radial corneal pocket. A slow release prostaglandin pellet was placed in the pocket to induce neovascularization. Test compound was administered i.p. for five days, at which time the animals were sacrificed. The effect of the test compound is evaluated by review of periodic photographs taken of the limbus, which can be used to calculate the area of neovascular response and, therefore, limbal neovascularization. A decreased area of neovascularization as compared with appropriate controls indicates the test compound was effective at decreasing or inhibiting neovascularization.

An angiogenesis model used to evaluate the effect of a test compound in preventing angiogenesis is described by Min et al. Canc. Res. 56:2428-2433 (1996). C57BL6 mice receive subcutaneous injections of a Matrigel mixture containing bFGF, as the angiogenesis-inducing agent, with and without the test compound. After five days, the animals are sacrificed and the Matrigel plugs, in which neovascularization can be visualized, are photographed. An experimental animal receiving Matrigel and an effective dose of test compound exhibits less vascularization than a control animal or an experimental animal receiving a less- or non-effective dose of compound.

An in vivo system designed to test compounds for their ability to limit the spread of primary tumors is described by Crowley et al., Proc. Natl. Acad. Sci. 90:5021-5025 (1993). Nude mice are injected with tumor cells (PC3) engineered to express CAT (chloramphenicol acetyltransferase). Compounds to be tested for their ability to decrease tumor size and/or metastases are administered to the animals, and subsequent measurements of tumor size and/or metastatic growths are made. In addition, the level of CAT detected in various organs provides an indication of the ability of the test compound to inhibit metastasis; detection of less CAT in tissues of a treated animal versus a control animal indicates less CAT-expressing cells migrated to that tissue.

In vivo experimental models designed to evaluate the inhibitory potential of a test serine protease inhibitors, using a tumor cell line F3II known to be highly invasive (see, e.g., Alonso et al., Breast Canc. Res. Treat. 40:209-223 (1996)) can be used. Alonso describes in vivo studies for toxicity determination, tumor growth, invasiveness, spontaneous metastasis, experimental lung metastasis, and an angiogenesis assay.

The CAM model (chick embryo chorioallantoic membrane model), first described by L. Ossowski in 1998 (J. Cell Biol. 107:2437-2445 (1988)), provides another method for evaluating the inhibitory activity of a test compound. In the CAM model, tumor cells invade through the chorioallantoic membrane. Administration of several serine protease inhibitors resulted in less or no invasion of the tumor cells through the membrane. Thus, the CAM assay is performed with CAM and tumor cells in the presence and absence of various concentrations of test compound. The invasiveness of tumor cells is measured under such conditions to provide an indication of the compound's inhibitory activity. A compound having inhibitory activity correlates with less tumor invasion.

The CAM model also is used in a standard assay of angiogenesis (i.e., effect on formation of new blood vessels (see, e.g., Brooks et al. Methods in Molecular Biology 129:257-269 (1999)). In this model, a filter disc containing an angiogenesis inducer, such as basic fibroblast growth factor (bFGF) is placed onto the CAM. Diffusion of the cytokine into the CAM induces local angiogenesis, which can be measured in several ways such as by counting the number of blood vessel branch points within the CAM directly below the filter disc. The ability of identified compounds to inhibit cytokine-induced angiogenesis can be tested using this model. A test compound can either be added to the filter disc that contains the angiogenesis inducer, be placed directly on the membrane or be administered systemically. The extent of new blood vessel formation in the presence and/or absence of test compound can be compared using this model. The formation of fewer new blood vessels in the presence of a test compound would be indicative of anti-angiogenesis activity. A demonstration of anti-angiogenesis activity for inhibitors of a CVSP16 polypeptide is indicative of a role in angiogenesis for that SP protein.

### b. Known Serine Protease Inhibitors

Compounds for screening can be serine protease inhibitors, which can be tested for their ability to inhibit the activity of a CVSP16. Exemplary serine protease inhibitors for use in the screening assays include, but are not limited to: Serine Protease Inhibitor 3 (SPI-3) (Chen, et al. Cytokine, 11:856-862 (1999)); Aprotinin (Iijima, R., et al., J. Biochem. (Tokyo) 126:912-916 (1999)); Kazal-type serine protease inhibitor-like proteins (Niimi, et al. Eur. J. Biochem., 266:282-292 (1999)); Kunitz-type serine protease inhibitor (Ravichandran, S., et al., Acta Crystallogr. D. Biol. Crystallogr., 55:1814-1821 (1999)); Tissue factor pathway inhibitor-2/Matrix-associated serine protease inhibitor (TFPI-2/MSPI), (Liu, Y. et al. Arch. Biochem. Biophys. 370:112-8 (1999)); Bukunin (Cui, C. Y. et al. J. Invest. Dermatol. 113:182-8 (1999)); Nafmostat mesilate (Ryo, R. et al. Vox Sang. 76:241-6 (1999)); TPCK (Huang et al. Oncogene 18:3431-3439 (1999)); A synthetic cotton-bound serine protease inhibitor (Edwards et al. Wound Repair Regen. 7:106-18 (1999)); FUT-175 (Sawada, M. et al. Stroke 30:644-50 (1999)); Combination of serine protease inhibitor FUT-0175 and thromboxane synthetase inhibitor OKY-046 (Kaminogo et al. Neurol. Med. Chir. (Tokyo) 38:704-8; discussion 708-9 (1998)); The rat serine protease inhibitor 2.1 gene (LeCam, A., et al., Biochem. Biophys. Res. Commun., 253:311-4 (1998)); A new intracellular serine protease inhibitor expressed in the rat pituitary gland complexes with granzyme B (Hill et al. FEBS Lett. 440:361-4 (1998)); 3,4-Dichloroisocoumarin (Hammed et al. Proc. Soc. Exp. Biol. Med., 219:132-7 (1998)); LEX032 (Bains et al. Eur. J. Pharmacol. 356:67-72 (1998)); N-tosyl-L-phenylalanine chloromethyl ketone (Dryjanski et al. Biochemistry 37:14151-6 (1998)); Mouse gene for the serine protease inhibitor neuroserpin (P112) (Berger et al. Gene, 214:25-33 (1998)); Rat serine protease inhibitor 2.3 gene (Paul et al. Eur. J. Biochem. 254:538-46 (1998)); Ecotin (Yang et al. J. Mol. Biol. 279:945-57 (1998)); A 14 kDa plant-related serine protease inhibitor (Roch et al. Dev. Comp. Immunol. 22(1):1-12 (1998)); Matrix-associated serine protease inhibitor TFPI-2/33 kDa MSPI (Rao et al. Int. J. Cancer 76:749-56 (1998)); ONO-3403 (Hiwasa et al. Cancer Lett. 126:221-5 (1998)); Bdellastasin (Moser et al. Eur. J. Biochem. 253:212-20 (1998)); Bikunin (Xu et al. J. Mol. Biol. 276:955-66 (1998)); Nafamostat mesilate (Meligren et al. Thromb. Haemost. 79:342-7 (1998)); The growth hormone dependent serine protease inhibitor, Spi 2.1 (Maake et al. Endocrinology 138:5630-6 (1997)); Growth factor activator inhibitor type 2, a Kunitz-type serine protease inhibitor (Kawaguchi et al. J. Biol. Chem., 272:27558-64 (1997)); Heat-stable serine protease inhibitor protein from ovaries of the desert locust, Schistocerga gregaria (Hamdaoui et al. Biochem. Biophys. Res. Commun. 238:357-60 (1997)); Human placental Hepatocyte growth factor activator inhibitor, a Kunitz-type serine protease inhibitor (Shimomura et al. J. Biol. Chem. 272:6370-6 (1997)); FUT-187, oral serine protease inhibitor (Shiozaki et al. Gan To Kaguku Ryoho, 23(14): 1971-9 (1996)); Extracellular matrix-associated serine protease inhibitors (Mr 33,000, 31,000, and 27,000 (Rao, C. N., et al., Arch. Biochem. Biophys., 335:82-92 (1996)); An irreversible isocoumarin serine protease inhibitor (Palencia, D. D., et al., Biol. Reprod., 55:536-42 (1996)); 4-(2-aminoethyl)-benzenesulfonyl fluoride (AEBSF) (Nakabo et al. J. Leukoc. Biol. 60:328-36 (1996)); Neuroserpin (Osterwalder, T., et al., EMBO J. 15:2944-53 (1996)); Human serine protease inhibitor alpha-1-antitrypsin (Forney et al. J. Parasitol. 82:496-502 (1996)); Rat serine protease inhibitor 2.3 (Simar-Blanchet, A. E., et al., Eur. J. Biochem., 236:638-48 (1996)); Gebaxate mesilate (parodi, F., et al., J. Cardiothorac. Vasc. Anesth. 10:235-7 (1996)); Recombinant serine protease inhibitor, CPTI II (Stankiewicz, M., et al., (Acta Biochim. Pol. 43(3):525-9 (1996)); A cysteine-rich serine protease inhibitor (Guamerin II) (Kim, D. R., et al., J. Enzym. Inhib., 10:81-91 (1996)); diisopropylfluorophosphate (Lundqvist, H., et al., Inflamm. Res. 44(12):510-7 (1995)); Nexin 1 (Yu, D. W., et al., J. Cell Sci. 108(Pt 12):3867-74 (1995)); LEX032 (Scalia, R., et al., Shock 4(4):251-6 (1995)); Protease nexin I (Houenou, L. J., et al., Proc. Natl. Acad. Sci. U.S.A. 92(3):895-9 (1995)); Chymase-directed serine protease inhibitor (Woodard S. L., et al., J. Immunol. 153(11):5016-25 (1994)); N-alpha-tosyl-L-lysyl-chloromethyl ketone (TLCK) (Bourinbaiar, A. S., et al., Cell Immunol. 155(1):230-6 (1994)); Smpi56 (Ghendler, Y., et al., Exp. Parasitol. 78(2): 121-31 (1994)); Schistosoma haematobium serine protease inhibitor (Blanton, R. E., et al., Mol. Biochem. Parasitol. 63(1):1-1 (1994)); Spi-1 (Warren, W. C., et al., Mol. Cell Endocrinol. 98(1):27-32 (1993)); TAME (Jessop, J. J., et al., Inflammation 17(5):613-31 (1993)); Antithrombin III (Kalaria, R. N., et al., Am. J. Pathol. 143(3):886-93 (1993)); FOY-305 (Ohkoshi, M., et al., Anticancer Res. 13(4):963-6 (1993)); Camostat mesilate (Senda, S., et al, Intern. Med. 32(4):350-4 (1993)); Pigment epithelium-derived factor (Steele, F. R., et al., Proc. Natl. Acad. Sci. U.S.A. 90(4):1526-30 (1993)); Antistasin (Holstein, T. W., et al., FEBS Lett. 309(3):288-92 (1992)); the vaccinia virus K2L gene encodes a serine protease inhibitor (Zhou, J., et al., Virology 189(2):678-86 (1992)); Bowman-Birk serine-protease inhibitor (Werner, M. H., et al., J. Mol. Biol. 225(3):873-89 (1992); FUT-175 (Yanamoto, H., et al., Neurosurgery 30(3):358-63 (1992)); FUT-175; (Yanamoto, H., et al., Neurosurgery 30(3):351-6, discussion 356-7 (1992)); PAI-I (Yreadwell, B. V., et al., J. Orthop. Res. 9(3):309-16 (1991)); 3,4-Dichloroisocoumarin (Rusbridge, N. M., et al., FEBS Lett. 268(1):133-6 (1990)); Alpha 1-antichymotrypsin (Lindmark, B. E., et al., Am. Rev. Respir. Des. 141(4 Pt 1):884-8 (1990)); P-toluenesulfonyl-L-arginine methyl ester (TAME) (Scuderi, P., J. Immunol., 143(1):168-73 (1989)); Alpha 1-antichymotrypsin (Abraham, C. R., et al. Cell 52(4):487-501 (1988)); Contrapsin (Modha, J., et al., Parasitology 96 (Pt 1):99-109 (1988)); Alpha 2-antiplasmin (Holmes, W. E., et al., J. Biol. Chem. 262(4):1659-64 (1987)); 3,4-dichloroisocoumarin (Harper, J. W., et al., Biochemistry 24(8):1831-41 (1985)); Diisopropylfluorophosphate (Tsutsui, K., et al, Biochem. Biophys. Res. Commun. 123(1):271-7 (1984)); Gabexate mesilate (Hesse, B., et al., Pharmacol. Res. Commun. 16(7):637-45 (1984)); Phenyl methyl sulfonyl fluoride (Dufer, J., et al., Scand. J. Haematol. 32(1):25-32 (1984)); Protease inhibitor C1-2 (McPhalen, C. A., et al., J. Mol. Biol. 168(2):445-7 (1983)); Phenylmethylsulfonyl fluoride (Sekar et al., Biochem. Biophys. Res. Commun., 89(2):474-8 (1979)); PGE1 (Feinstein et al., Prostaglandins 14(6):1075-93 (1977)).

### C. Combinatorial Libraries and Other Libraries

The source of compounds for the screening assays, can be libraries, including, but are not limited to, combinatorial libraries. Methods for synthesizing combinatorial libraries and characteristics of such combinatorial libraries are known in the art (See generally, Combinatorial Libraries: Synthesis, Screening and Application Potential (Cortese Ed.) Walter de Gruyter, Inc., 1995; Tietze and Lieb, Curr. Opin. Chem. Biol. 2(3):363-71 (1998); Lam, Anticancer Drug Des. 12(3):145-67 (1997); Blaney and Martin, Curr. Opin. Chem. Biol. 1(1):54-9 (1997); and Schultz and Schultz, Biotechnol. Prog. 12(6):729-43 (1996)).

Methods and strategies for generating diverse libraries, primarily peptide- and nucleotide-based oligomer libraries, have been developed using molecular biology methods and/or simultaneous chemical synthesis methodologies (see, e.g., Dower et al., Annu. Rep. Med. Chem. 26:271-280 (1991); Fodor et al., Science 251:767-773 (1991); Jung et al., Angew. Chem. Ind. Ed. Engl. 31:367-383 (1992); Zuckerman et al., Proc. Natl. Acad. Sci. USA 89:4505-4509 (1992); Scott et al., Science 249:386-390 (1990); Devlin et al., Science 249:404-406 (1990); Cwirla et al., Proc. Natl. Acad. Sci. USA 87:6378-6382 (1990); and Gallop et al., J. Medicinal Chemistry 37:1233-1251 (1994)). The resulting combinatorial libraries potentially contain millions of compounds that can be screened to identify compounds that exhibit a selected activity.

The libraries fall into roughly three categories: fusion-protein-displayed peptide libraries in which random peptides or proteins are presented on the surface of phage particles or proteins expressed from plasmids; support-bound synthetic chemical libraries in which individual compounds or mixtures of compounds are presented on insoluble matrices, such as resin beads (see, e.g., Lam et al., Nature 354:82-84 (1991)) and cotton supports (see, e.g., Eichler et al., Biochemistry 32:11035-11041 (1993)); and methods in which the compounds are used in solution (see, e.g., Houghten et al., Nature 354:84-86 (1991); Houghten et al., BioTechniques 313:412-421 (1992); and Scott et al., Curr. Opin. Biotechnol. 5:40-48 (1994)). There are numerous examples of synthetic peptide and oligonucleotide combinatorial libraries and there are many methods for producing libraries that contain non-peptidic small organic molecules. Such libraries can be based on a basis set of monomers that are combined to form mixtures of diverse organic molecules or that can be combined to form a library based upon a selected pharmacophore monomer.

Either a random or a deterministic combinatorial library can be screened by the presently disclosed and/or claimed screening methods. In either of these two libraries, each unit of the library is isolated and/or immobilized on a solid support. In the deterministic library, one knows a priori a particular unit's location on each solid support. In a random library, the location of a particular unit is not known a priori although each site still contains a single unique unit. Many methods for preparing libraries are known to those of skill in this art (see, e.g., Geysen et al., Proc. Natl. Acad. Sci. USA 81:3998-4002 (1984), Houghten et al., Proc. Natl. Acad. Sci. USA 81:5131-5135 (1985)).

Combinatorial libraries generated by any techniques known to those of skill in the art are contemplated (see, e.g., Table 1 of Schultz and Schultz, Biotechnol. Prog. 12(6):729-43 (1996)) for screening; Bartel et al., Science 261:1411-1418 (1993); Baumbach et al. BioPharm (May):24-35 (1992); Bock et al. Nature 355:564-566 (1992); Borman, S., Combinatorial chemists focus on small molecules molecular recognition, and automation, Chem. Eng. News 2(12):29 (1996); Boublik, et al., Eukaryotic Virus Display: Engineering the Major Surface Glycoproteins of the Autographa Californica Nuclear Polyhedrosis Virus (ACNPV) for the Presentation of Foreign Proteins on the Virus Surface, Bio/Technology 13:1079-1084 (1995); Brenner, et al., Encoded Combinatorial Chemistry, Proc. Natl. Acad. Sci. U.S.A. 89:5381-5383 (1992); Caflisch, et al., Computational Combinatorial Chemistry for De Novo Ligand Design: Review and Assessment, Perspect. Drug Discovery Des. 3:51-84 (1995); Cheng, et al., Sequence-Selective Peptide Binding with a Peptido-A,B-trans-steroidal Receptor Selected from an Encoded Combinatorial Library, J. Am. Chem. Soc. 118:1813-1814 (1996); Chu, et al., Affinity Capillary Electrophoresis to Identify the Peptide in A Peptide Library that Binds Most Tightly to Vancomycin, J. Org. Chem. 58:648-652 (1993); Clackson, et al., Making Antibody Fragments Using Phage Display Libraries, Nature 352:624-628 (1991); Combs, et al., Protein Structure-Based Combinatorial Chemistry: Discovery of Non-Peptide Binding Elements to Src SH3 Domain, J. Am. Chem. Soc. 118:287-288 (1996); Cwirla, et al., Peptides On Phage: A Vast Library of Peptides for Identifying Ligands, Proc. Natl. Acad. Sci. U.S.A. 87:6378-6382 (1990); Ecker, et al., Combinatorial Drug Discovery: Which Method will Produce the Greatest Value, Bio/Technology 13:351-360 (1995); Ellington, et al., In Vitro Selection of RNA Molecules That Bind Specific Ligands, Nature, 346:818-822 (1990); Ellman, J. A., Variants of Benzodiazepines, J. Am. Chem. Soc., 114:10997 (1992); Erickson, et al., The Proteins; Neurath, H., Hill, R. L., Eds.: Academic: New York, 1976; pp. 255-257; Felici, et al., J. Mol. Biol. 222:301-310 (1991); Fodor, et al., Light-Directed, Spatially Addressable Parallel Chemical Synthesis, Science 251:767-773 (1991); Francisco, et al., Transport and Anchoring of Beta-Lactamase to the External Surface of E. Coli., Proc. Natl. Acad. Sci. U.S.A. 89:2713-2717 (1992); Georgiou, et al., Practical Applications of Engineering Gram-Negative Bacterial Cell Surfaces, TIBTECH 11:6-10 (1993); Geysen, et al., Use of peptide synthesis to probe viral antigens for epitopes to a resolution of a single amino acid, Proc. Natl. Acad. Sci. U.S.A. 81:3998-4002 (1984); Glaser, et al., Antibody Engineering by Condon-Based Mutagenesis in a Filamentous Phage Vector System, J. Immunol. 149:3903-3913 (1992); Gram, et al., In vitro selection and affinity maturation of antibodies from a naive combinatorial immunoglobulin library, Proc. Natl. Acad. Sci. 89:3576-3580 (1992); Han, et al., Liquid-Phase Combinatorial Synthesis, Proc. Natl. Acad. Sci. U.S.A. 92:6419-6423 (1995); Hoogenboom, et al., Multi-Subunit Proteins on the Surface of Filamentous Phage: Methodologies for Displaying Antibody (Fab) Heavy and Light Chains, Nucleic Acids Res. 19:4133-4137 (1991); Houghten, et al., General Method for the Rapid Solid-Phase Synthesis of Large Numbers of Peptides: Specificity of Antigen-Antibody Interaction at the Level of Individual Amino Acids, Proc. Natl. Acad. Sci. U.S.A. 82:5131-5135 (1985); Houghten, et al., The Use of Synthetic Peptide Combinatorial Libraries for the Determination of Peptide Ligands in Radio-Receptor Assays-Opioid-Peptides, Bioorg. Med. Chem. Lett. 3:405-412 (1993); Houghten, et al., Generation and Use of Synthetic Peptide Combinatorial Libraries for Basic Research and Drug Discovery, Nature 354:84-86 (1991); Huang, et al., Discovery of New Ligand Binding Pathways in Myoglobin by Random Mutagenesis, Nature Struct. Biol. 1:226-229 (1994); Huse, et al., Generation of a Large Combinatorial Library of the Immunoglobulin Repertoire In Phage Lambda, Science 246:1275-1281 (1989); Janda, K. D., New Strategies for the Design of Catalytic Antibodies, Biotechnol. Prog. 6:178-181 (1990); Jung, et al., Multiple Peptide Synthesis Methods and Their Applications, Angew. Chem. Int. Ed. Engl. 31:367-486 (1992); Kang, et al., Linkage of Recognition and Replication Functions By Assembling Combinatorial Antibody Fab Libraries Along Phage Surfaces, Proc. Natl. Acad. Sci. U.S.A. 88:4363-4366 (1991a); Kang, et al., Antibody Redesign by Chain Shuffling from Random Combinatorial Immunoglobulin Libraries, Proc. Natl. Acad. Sci. U.S.A., 88:11120-11123 (1991b); Kay, et al., An M13 Phage Library Displaying Random 38-Amino-Acid-Peptides as a Source of Novel Sequences with Affinity to Selected Targets Genes, Gene 128:59-65 (1993); Lam, et al., A new type of synthetic peptide library for identifying ligand-binding activity, Nature 354:82-84 (1991) (published errata in Nature 358:434 (1992) and Nature 360:768 (1992); Lebl, et al., One Bead One Structure Combinatorial Libraries, Biopolymers (Pept. Sci.) 37:177-198 (1995); Lerner, et al., Antibodies without Immunization, Science 258:1313-1314 (1992); Li, et al., Minimization of a Polypeptide Hormone, Science 270:1657-1660 (1995); Light, et al., Display of Dimeric Bacterial Alkaline Phosphatase on the Major Coat Protein of Filamentous Bacteriophage, Bioorg. Med. Chem. Lett. 3:1073-1079 (1992); Little, et al., Bacterial Surface Presentation of Proteins and Peptides: An Alternative to Phage Technology, Trends Biotechnol. 11:3-5 (1993); Marks, et al., By-Passing Immunization. Human Antibodies from V-Gene Libraries Displayed on Phage, J. Mol. Biol., 222:581-597 (1991); Matthews, et al., Substrate Phage: Selection of Protease Substrates by Monovalent Phage Display, Science 260:1113-1117 (1993); McCafferty, et al., Phage Enzymes: Expression and Affinity Chromatography of Functional Alkaline Phosphatase on the Surface of Bacteriophage, Protein Eng. 4:955-961 (1991); Menger, et al., Phosphatase Catalysis Developed Via Combinatorial Organic Chemistry, J. Org. Chem. 60:6666-6667 (1995); Nicolaou, et al., Angew. Chem. Int. Ed. Engl. 34:2289-2291 (1995); Oldenburg, et al., Peptide Ligands for A Sugar-Binding Protein Isolated from a Random Peptide Library, Proc. Natl. Acad. Sci. U.S.A. 89:5393-5397 (1992); Parmley, et al., Antibody-Selectable Filamentous fd Phage Vectors: Affinity Purification of Target Genes, Genes 73:305-318 (1988); Pinilla, et al., Synthetic Peptide Combinatorial Libraries (SPCLS)—Identification of the Antigenic Determinant of Beta-Endorphin Recognized by Monoclonal Antibody-3E7, Gene 128:71-76 (1993); Pinilla, et al., Review of the Utility of Soluble Combinatorial Libraries, Biopolymers 37:221-240 (1995); Pistor, et al., Expression of Viral Hemaegglutinin On the Surface of E. Coli., Klin. Wochenschr. 66:110-116 (1989); Pollack, et al., Selective Chemical Catalysis by an Antibody, Science 234:1570-1572 (1986); Rigler, et al., Fluorescence Correlations, Single Molecule Detection and Large Number Screening: Applications in Biotechnology, J. Biotechnol. 41:177-186 (1995); Sarvetnick, et al., Increasing the Chemical Potential of the Germ-Line Antibody Repertoire, Proc. Natl. Acad. Sci. U.S.A. 90:4008-4011 (1993); Sastry, et al., Cloning of the Immunological Repertiore in Escherichia Coli for Generation of Monoclonal Catalytic Antibodies: Construction of a Heavy Chain Variable Region-Specific cDNA Library, Proc. Natl. Acad. Sci. U.S.A. 86:5728-5732 (1989); Scott, et al., Searching for Peptide Ligands with an Epitope Library, Science 249:386-390 (1990); Sears, et al., Engineering Enzymes for Bioorganic Synthesis: Peptide Bond Formation, Biotechnol. Prog. 12:423-433 (1996); Simon, et. al., Peptides: A Modular Approach to Drug Discovery, Proc. Natl. Acad. Sci. U.S.A. 89:9367-9371 (1992); Still, et al., Discovery of Sequence-Selective Peptide Binding by Synthetic Receptors Using Encoded Combinatorial Libraries, Acc. Chem. Res. 29:155-163 (1996); Thompson, et al., Synthesis and Applications of Small Molecule Libraries, Chem. Rev. 96:555-600 (1996); Tramontano, et al., Catalytic Antibodies, Science 234:1566-1570 (1986); Wrighton, et al., Small Peptides as Potent Mimetics of the Protein Hormone Erythropoietin, Science 273:458-464 (1996); York, et al., Combinatorial mutagenesis of the reactive site region in plasminogen activator inhibitor I, J. Biol. Chem. 266:8595-8600 (1991); Zebedee, et al., Human Combinatorial Antibody Libraries to Hepatitis B Surface Antigen, Proc. Natl. Acad. Sci. U.S.A. 89:3175-3179 (1992); Zuckerman, et al., Identification of Highest-Affinity Ligands by Affinity Selection from Equimolar Peptide Mixtures Generated by Robotic Synthesis, Proc. Natl. Acad. Sci. U.S.A. 89:4505-4509 (1992).

For example, peptides that bind to a CVSP16 polypeptide or a protease domain of a SP protein can be identified using phage display libraries. In an exemplary embodiment, this method can include a) contacting phage from a phage library with the CVSP16 polypeptide or a protease domain thereof; (b) isolating phage that bind to the protein; and (c) determining the identity of at least one peptide coded by the isolated phage to identify a peptide that binds to a CVSP16 polypeptide.

H. Modulators of the Activity of CVSP16 Polypeptides

Provided herein are compounds, identified by screening or produced using the CVSP16 polypeptide or protease domain in other screening methods, that modulate the activity of a CVSP16. These compounds act by directly interacting with the CVSP16 polypeptide or by altering transcription or translation thereof. Such molecules include, but are not limited to, antibodies that specifically react with a CVSP16 polypeptide, particularly with the protease domain thereof, antisense nucleic acids or double-stranded RNA (dsRNA) such as RNAi, including those that contain modified nucleic acids, that alter expression of the CVSP16 polypeptide, peptide mimetics and other such compounds.

1. Antibodies

Antibodies, including polyclonal and monoclonal antibodies, that specifically bind to a CVSP16 polypeptide provided herein, including antibodies to single chain protease domains thereof, or to activated forms of a single-chain protease domain, or to full-length activaed or zymogen forms of the polypeptide or to other portions of a CVSP16 are provided. Generally, the antibody is a monoclonal antibody, and typically the antibody specifically binds to a protease domain of the CVSP16 polypeptide.

Provided are antibodies that specifically bind to any domain of CVSP16, and antibodies that specifically bind to two-chain and/or three-chain thereof. Also provided are antibodies that specifically bind to an active site or active site cleft of zymogen and activated forms. Neutralizing antibodies are also provided.

The CVSP16 polypeptide and domains, fragments, homologs and derivatives thereof can be used as immunogens to generate antibodies that specifically bind CVSP16 polypeptides and portions thereof. Such antibodies include but are not limited to polyclonal, monoclonal, chimeric, single chain, Fab fragments, and a Fab expression library. In a specific embodiment, antibodies to human CVSP16 polypeptide are produced. In another embodiment, complexes formed from fragments of CVSP16 polypeptide, which fragments contain the serine protease domain, are used as immunogens for antibody production.

Antibodies provided herein include, but are not limited to, monoclonal and polyclonal antibodies. They include antibodies that inhibits catalytic activity of a CVSP16 polypeptide provided herein and/or a ligand or substrate binding activity of the polypeptide. Also included are antibodies that specifically bind to a single-chain protease domain 1 (PD1) of a CVSP16 polypeptide and/or two-chain PD1 and antibodies and antibodies that specifically bind to a single-chain protease domain 1 (PD2) of a CVSP16 polypeptide and/or two chain PD2. Included are antibodies that specifically that bind to a single-chain form and/or to two-chain and/or three-chain forms of a CVSP16 polypeptide.

Various procedures known in the art can be used for the production of polyclonal antibodies to CVSP16 polypeptide, its domains, derivatives, fragments or analogs. For production of the antibody, various host animals can be immunized by injection with the native CVSP16 polypeptide or a synthetic version, or a derivative of the foregoing, such as a cross-linked CVSP16 polypeptide. Such host animals include but are not limited to rabbits, mice, rats, chickens and other animals. Various adjuvants can be used to increase the immunological response, depending on the host species, and include but are not limited to Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as bacille Calmette-Guerin (BCG) and corynebacterium parvum.

For preparation of monoclonal antibodies directed towards a CVSP16 polypeptide or domains, derivatives, fragments or analogs thereof, any technique that provides for the production of antibody molecules by continuous cell lines in culture can be used. Such techniques include but are not restricted to the hybridoma technique originally developed by Kohler and Milstein (Nature 256:495-497 (1975)), the trioma technique, the human B-cell hybridoma technique (Kozbor et al., Immunology Today 4:72 (1983)), and the EBV hybridoma technique to produce human monoclonal antibodies (Cole et al., in Monoclonal Antibodies and Cancer Therapy, Alan R. Liss, Inc., pp. 77-96 (1985)). For example, immortalized cell lines that secrete the desired monoclonal antibodies are provided. The immortalized cell lines secreting the desired antibodies are screened by immunoassays in which the antigen is the peptide hapten, polypeptide or protein. When the appropriate immortalized cell culture secreting the desired antibody is identified, the cells can be cultured either in vitro or by production in vivo via ascites fluid.

Monoclonal antibodies can be produced by other methods, such as in germ-free animals utilizing recent technology (PCT/US90/02545). Human antibodies can be used and can be obtained by using human hybridomas (Cote et al., Proc. Natl. Acad. Sci. USA 80:2026-2030 (1983)), or by transforming human B cells with EBV virus in vitro (Cole et al., in Monoclonal Antibodies and Cancer Therapy, Alan R. Liss, Inc., pp. 77-96 (1985)). Techniques developed for the production of “chimeric antibodies” (Morrison et al., Proc. Natl. Acad. Sci. USA 81:6851-6855 (1984); Neuberger et al., Nature 312:604-608 (1984); Takeda et al., Nature 314:452-454 (1985)) by splicing the genes from a mouse antibody molecule specific for the CVSP16 polypeptide together with genes from a human antibody molecule of appropriate biological activity can be used.

Techniques described for the production of single chain antibodies (U.S. Pat. No. 4,946,778) can be adapted to produce CVSP16 polypeptide-specific single chain antibodies. An additional embodiment uses the techniques described for the construction of Fab expression libraries (Huse et al., Science 246:1275-1281 (1989)) to allow rapid and easy identification of monoclonal Fab fragments with the desired specificity for CVSP16 polypeptide or domains, derivatives, or analogs thereof. Non-human antibodies can be “humanized” by known methods (see, e.g., U.S. Pat. No. 5,225,539).

Antibody fragments that specifically bind to CVSP16 polypeptide or epitopes thereof can be generated by techniques known in the art. For example, such fragments include but are not limited to: the F(ab′)2 fragment, which can be produced by pepsin digestion of the antibody molecule; the Fab′ fragments that can be generated by reducing the disulfide bridges of the F(ab′)2 fragment; the Fab fragments that can be generated by treating the antibody molecule with papain and a reducing agent; and Fv fragments.

In the production of antibodies, screening for the desired antibody can be accomplished by techniques known in the art, e.g., ELISA (enzyme-linked immunosorbent assay). To select antibodies specific to a particular domain of the CVSP16 polypeptide one can assay generated hybridomas for a product that binds to the fragment of the CVSP16 polypeptide that contains such a domain.

The foregoing antibodies can be used in methods known in the art relating to the localization and/or quantitation of CVSP16 polypeptide proteins, e.g., for imaging these proteins, measuring levels thereof in appropriate physiological samples, in, for example, diagnostic methods. In another embodiment, anti-CVSP16 polypeptide antibodies, or fragments thereof, containing the binding domain are used as therapeutic agents.

2. Peptides, Polypeptides and Peptide Mimetics

Provided herein are methods for identifying molecules that bind to and modulate the activity of SP proteins. Included among molecules that bind to SPs, particularly the single chain protease domain or catalytically active fragments thereof, are peptides, polypeptides and peptide mimetics, including cyclic peptides. Peptide mimetics are molecules or compounds that mimic the necessary molecular conformation of a ligand or polypeptide for specific binding to a target molecule such as a CVSP16 polypeptide. In an exemplary embodiment, the peptides, polypeptides or peptide mimetics bind to the protease domain of the CVSP16 polypeptide. Such peptides and peptide mimetics include those of antibodies that specifically bind to a CVSP16 polypeptide and, typically, bind to the protease domain of a CVSP16 polypeptide. The peptides, polypeptides and peptide mimetics identified by methods provided herein can be agonists or antagonists of CVSP16 polypeptides.

Such peptides and peptide mimetics are useful for diagnosing, treating, preventing, and screening for a disease or disorder associated with CVSP16 polypeptide activity in a mammal. In addition, the peptides and peptide mimetics are useful for identifying, isolating, and purifying molecules or compounds that modulate the activity of a CVSP16 polypeptide, or specifically bind to a CVSP16 polypeptide, generally the protease domain of a CVSP16 polypeptide. Low molecular weight peptides and peptide mimetics can have strong binding properties to a target molecule, e.g., a CVSP16 polypeptide and/or the protease domain of a CVSP16 polypeptide.

Peptides, polypeptides and peptide mimetics that bind to CVSP16 polypeptides as described herein can be administered to mammals, including humans, to modulate CVSP16 polypeptide activity. Thus, methods for therapeutic treatment and prevention of neoplastic diseases include administering a peptide, polypeptides or peptide mimetic compound in an amount sufficient to modulate such activity are provided. Thus, also provided herein are methods for treating a subject having such a disease or disorder in which a peptide, polypeptides or peptide mimetic compound is administered to the subject in a therapeutically effective dose or amount.

Compositions containing the peptides, polypeptides or peptide mimetics can be administered for prophylactic and/or therapeutic treatments. In therapeutic applications, compositions can be administered to a patient already suffering from a disease, as described above, in an amount sufficient to cure or at least partially arrest the symptoms of the disease and its complications. Amounts effective for this use will depend on the severity of the disease and the weight and general state of the patient and can be empirically determined.

In prophylactic applications, compositions containing the peptides, polypeptides and peptide mimetics are administered to a patient susceptible to or otherwise at risk of a particular disease. Such an amount is defined to be a “prophylactically effective dose”. In this use, the precise amounts again depend on the patient's state of health and weight. Accordingly, the peptides, polypeptides and peptide mimetics that bind to a CVSP16 polypeptide can be used to prepare pharmaceutical compositions containing, as an active ingredient, at least one of the peptides or peptide mimetics in association with a pharmaceutical carrier or diluent. The compounds can be administered, for example, by oral, pulmonary, parental (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration (see, e.g., International PCT application Nos. WO 93/25221 and WO 94/17784; and European Patent Application 613,683).

Peptides, polypeptides and peptide mimetics that bind to CVSP16 polypeptides are useful in vitro as tools for understanding the biological role of CVSP16 polypeptides, including the evaluation of the many factors thought to influence, and be influenced by, the production of CVSP16 polypeptide. Such peptides, polypeptides and peptide mimetics also are useful in the development of other compounds that bind to and modulate the activity of a CVSP16 polypeptide, because such compounds provide important information on the relationship between structure and activity that should facilitate such development.

The peptides, polypeptides and peptide mimetics are also useful as competitive binders in assays to screen for new CVSP16 polypeptides or CVSP16 polypeptide agonists. In such assay embodiments, the compounds can be used without modification or can be modified in a variety of ways; for example, by labeling, such as covalently or non-covalently joining a moiety which directly or indirectly provides a detectable signal. In any of these assays, the materials thereto can be labeled either directly or indirectly. Possibilities for direct labeling include label groups such as: radiolabels such as ¹²⁵I enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups. The compounds also can include spacers or linkers in cases where the compounds are to be attached to a solid support.

Moreover, based on their ability to bind to a CVSP16 polypeptide, the peptides, polypeptides and peptide mimetics can be used as reagents for detecting CVSP16 polypeptides in living cells, fixed cells, in biological fluids, in tissue homogenates and in purified, natural biological materials. For example, by labeling such peptides, polypeptides and peptide mimetics, cells having CVSP16 polypeptides can be identified. In addition, based on their ability to bind a CVSP16 polypeptide, the peptides, polypeptides and peptide mimetics can be used in in situ staining, FACS (fluorescence-activated cell sorting), Western blotting, ELISA and other analytical protocols. Based on their ability to bind to a CVSP16 polypeptide, the peptides, polypeptides and peptide mimetics can be used in purification of CVSP16 polypeptides or in purifying cells expressing the CVSP16 polypeptide, e.g., a polypeptide encoding the protease domain of a CVSP16 polypeptide.

The peptides, polypeptides and peptide mimetics also can be used as commercial reagents for various medical research and diagnostic uses. The activity of the peptides and peptide mimetics can be evaluated either in vitro or in vivo in one of the numerous models described in McDonald (1992) Am. J. of Pediatric Hematology/Oncology, 14:8-21.

3. Peptide, Polypeptides and Peptide Mimetic Therapy

Peptide analogs are commonly used in the pharmaceutical industry as non-peptide drugs with properties analogous to those of the template peptide. These types of non-peptide compounds are termed “peptide mimetics” or “peptidomimetics” (Luthman et al., A Textbook of Drug Design and Development, 14:386-406, 2nd Ed., Harwood Academic Publishers (1996); Joachim Grante (1994) Angew. Chem. Int. Ed. Engl., 33:1699-1720; Fauchere (1986) J. Adv. Drug Res., 15:29; Veber and Freidinger (1985) TINS, p. 392; and Evans et al. (1987) J. Med. Chem. 30:1229). Peptide mimetics that are structurally similar to therapeutically useful peptides can be used to produce an equivalent or enhanced therapeutic or prophylactic effect. Preparation of peptidomimetics and structures thereof are known to those of skill in this art.

Systematic substitution of one or more amino acids of a consensus sequence with a D-amino acid of the same type (e.g., D-lysine in place of L-lysine) can be used to generate more stable peptides. In addition, constrained peptides containing a consensus sequence or a substantially identical consensus sequence variation can be generated by methods known in the art (Rizo et al. (1992) An. Rev. Biochem., 61:387, incorporated herein by reference); for example, by adding internal cysteine residues capable of forming intramolecular disulfide bridges which cyclize the peptide.

Those skilled in the art appreciate that modifications can be made to the peptides and mimetics without deleteriously effecting the biological or functional activity of the peptide. Further, the skilled artisan would know how to design non-peptide structures in three dimensional terms, that mimic the peptides that bind to a target molecule, e.g., a CVSP16 polypeptide or, generally, the protease domain of CVSP16 polypeptides (see, e.g., Eck and Sprang (1989) J. Biol. Chem., 26: 17605-18795).

When used for diagnostic purposes, the peptides and peptide mimetics can be labeled with a detectable label and, accordingly, the peptides and peptide mimetics without such a label can serve as intermediates in the preparation of labeled peptides and peptide mimetics. Detectable labels can be molecules or compounds, which when covalently attached to the peptides and peptide mimetics, permit detection of the peptide and peptide mimetics in vivo, for example, in a patient to whom the peptide or peptide mimetic has been administered, or in vitro, e.g., in a sample or cells. Suitable detectable labels are well known in the art and include, by way of example, radioisotopes, fluorescent labels (e.g., fluorescein), and the like. The particular detectable label employed is not critical and is selected to be detectable at non-toxic levels. Selection of the such labels is well within the skill of the art.

Covalent attachment of a detectable label to the peptide or peptide mimetic is accomplished by conventional methods well known in the art. For example, when the ¹²⁵I radioisotope is employed as the detectable label, covalent attachment of ¹²⁵I to the peptide or the peptide mimetic can be achieved by incorporating the amino acid tyrosine into the peptide or peptide mimetic and then iodinating the peptide (see, e.g., Weaner et al. (1994) Synthesis and Applications of Isotopically Labelled Compounds, pp. 137-140). If tyrosine is not present in the peptide or peptide mimetic, incorporation of tyrosine to the N or C terminus of the peptide or peptide mimetic can be achieved by well known chemistry. Likewise, ³²P can be incorporated onto the peptide or peptide mimetic as a phosphate moiety through, for example, a hydroxyl group on the peptide or peptide mimetic using conventional chemistry.

Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivatization (e.g., labeling) of peptidomimetics should not substantially interfere with the desired biological or pharmacological activity of the peptidomimetic.

Peptides, polypeptides and peptide mimetics that can bind to a CVSP16 polypeptide or the protease domain of CVSP16 polypeptides and/or modulate the activity thereof, or exhibit CVSP16 protease activity, can be used for treatment of neoplastic disease. The peptides, polypeptides and peptide mimetics can be delivered, in vivo or ex vivo, to the cells of a subject in need of treatment. Further, peptides which have CVSP16 polypeptide activity can be delivered, in vivo or ex vivo, to cells which carry mutant or missing alleles encoding the CVSP16 polypeptide gene. Any of the techniques described herein or known to the skilled artisan can be used for preparation and in vivo or ex vivo delivery of such peptides, polypeptides and peptide mimetics that are substantially free of other human proteins. For example, the peptides, polypeptides and peptide mimetics can be readily prepared by expression in a microorganism or synthesis in vitro.

The peptides or peptide mimetics can be introduced into cells, in vivo or ex vivo, by microinjection or by use of liposomes, for example. Alternatively, the peptides, polypeptides or peptide mimetics can be taken up by cells, in vivo or ex vivo, actively or by diffusion. In addition, extracellular application of the peptide, polypeptides or peptide mimetic can be sufficient to effect treatment of a neoplastic disease. Other molecules, such as drugs or organic compounds, that: 1) bind to a CVSP16 polypeptide or protease domain thereof; or 2) have a similar function or activity to a CVSP16 polypeptide or protease domain thereof, can be used in methods for treatment.

4. Rational Drug Design

The goal of rational drug design is to produce structural analogs of biologically active polypeptides or peptides of interest or of small molecules or peptide mimetics with which they interact (e.g., agonists and antagonists) in order to fashion drugs which are, e.g., more active or stable forms thereof; or which, for example, enhance or interfere with the function of a polypeptide in vivo (e.g., a CVSP16 polypeptide). In one approach, one first determines the three-dimensional structure of a protein of interest (e.g., a CVSP16 polypeptide or polypeptide having a protease domain) or, for example, of a CVSP16 polypeptide-ligand complex, by X-ray crystallography, by computer modeling or most typically, by a combination of approaches. Also, useful information regarding the structure of a polypeptide can be gained by modeling based on the structure of homologous proteins. In addition, peptides can be analyzed by an alanine scan. In this technique, an amino acid residue is replaced by Ala, and its effect on the peptide's activity is determined. Each of the amino acid residues of the peptide is analyzed in this manner to determine the important regions of the peptide.

Also, a polypeptide or peptide that binds to a CVSP16 polypeptide or, generally, the protease domain of a CVSP16 polypeptide, can be selected by a functional assay, and then the crystal structure of this polypeptide or peptide can be determined. The polypeptide can be, for example, an antibody specific for a CVSP16 polypeptide and/or the protease domain of a CVSP16 polypeptide. This approach can yield a pharmacophore upon which subsequent drug design can be based. Further, it is possible to bypass the crystallography altogether by generating anti-idiotypic polypeptides or peptides, (anti-ids) to a functional, pharmacologically active polypeptide or peptide that binds to a CVSP16 polypeptide or protease domain of a CVSP16 polypeptide. As a mirror image of a mirror image, the binding site of the anti-ids is expected to be an analog of the original target molecule, e.g., a CVSP16 polypeptide or polypeptide having a CVSP16 polypeptide. The anti-id could then be used to identify and isolate peptides from banks of chemically or biologically produced banks of peptides. Selected peptides would then act as the pharmacophore.

Thus, one can design drugs which have, e.g., improved activity or stability or which act as modulators (e.g., inhibitors, agonists, antagonists) of CVSP16 polypeptide activity, and are useful in the methods, particularly the methods for diagnosis, treatment, prevention, and screening of a neoplastic disease. By virtue of the availability of cloned CVSP16 polypeptide sequences, sufficient amounts of the CVSP16 polypeptide can be made available to perform such analytical studies as X-ray crystallography. In addition, the knowledge of the amino acid sequence of a CVSP16 polypeptide or the protease domain thereof, e.g., the protease domain encoded by the nucleotide sequence of SEQ ID No. 6, can provide guidance on computer modeling techniques in place of, or in addition to, X-ray crystallography.

### Methods of Identifying Peptides and Peptide Mimetics that Bind to CVSP16 Polypeptides

Peptides having a binding affinity to the CVSP16 polypeptide are provided herein (e.g., a CVSP16 polypeptide or a polypeptide having a protease domain of a CVSP16 polypeptide) and can be readily identified, for example, by random peptide diversity generating systems coupled with an affinity enrichment process. Specifically, random peptide diversity generating systems include the “peptides on plasmids” system (see, e.g., U.S. Pat. Nos. 5,270,170 and 5,338,665); the “peptides on phage” system (see, e.g., U.S. Pat. No. 6,121,238 and Cwirla, et al. (1990) Proc. Natl. Acad. Sci. U.S.A. 87:6378-6382); the “polysome system;” the “encoded synthetic library (ESL)” system; and the “very large scale immobilized polymer synthesis” system (see, e.g., U.S. Pat. No. 6,121,238; and Dower et al. (1991) An. Rep. Med. Chem. 26:271-280).

For example, using the procedures described above, random peptides can generally be designed to have a defined number of amino acid residues in length (e.g., 12). To generate the collection of oligonucleotides encoding the random peptides, the codon motif (NNK)x, where N is nucleotide A, C, G, or T (equimolar; depending on the methodology employed, other nucleotides can be employed), K is G or T (equimolar), and x is an integer corresponding to the number of amino acids in the peptide (e.g., 12) and can be used to specify any one of the 32 possible codons resulting from the NNK motif: 1 for each of 12 amino acids, 2 for each of 5 amino acids, 3 for each of 3 amino acids, and only one of the three stop codons. Thus, the NNK motif encodes all of the amino acids, encodes only one stop codon, and reduces codon bias.

The random peptides can be presented, for example, either on the surface of a phage particle, as part of a fusion protein containing either the pIII or the pVIII coat protein of a phage fd derivative (peptides on phage) or as a fusion protein with the LacI peptide fusion protein bound to a plasmid (peptides on plasmids). The phage or plasmids, including the DNA encoding the peptides, can be identified and isolated by an affinity enrichment process using immobilized CVSP16 polypeptide having a protease domain. The affinity enrichment process, sometimes called “panning,” typically involves multiple rounds of incubating the phage, plasmids, or polysomes with the immobilized CVSP16 polypeptide, collecting the phage, plasmids, or polysomes that bind to the CVSP16 polypeptide (along with the accompanying DNA or mRNA), and producing more of the phage or plasmids (along with the accompanying LacI-peptide fusion protein) collected.

### Characteristics of Peptides and Peptide Mimetics

Among the peptides, polypeptides and peptide mimetics for therapeutic application are those having molecular weights from about 250 to about 8,000 daltons. If such peptides are oligomerized, dimerized and/or derivatized with a hydrophilic polymer (e.g., to increase the affinity and/or activity of the compounds), the molecular weights of such peptides can be substantially greater and can range anywhere from about 500 to about 120,000 daltons, generally from about 8,000 to about 80,000 daltons. Such peptides can contain 9 or more amino acids that are naturally occurring or synthetic (non-naturally occurring) amino acids. One skilled in the art can determine the affinity and molecular weight of the peptides and peptide mimetics suitable for therapeutic and/or diagnostic purposes (e.g., see Dower et al., U.S. Pat. No. 6,121,238).

5. Methods of Preparing Peptides and Peptide Mimetics

Peptides and peptide mimetics can be designed, using a variety of methods, such as, for example, the “encoded synthetic library” or “very large scale immobilized polymer synthesis” systems (see, e.g., U.S. Pat. Nos. 5,925,525 and 5,902,723). Using the “encoded synthetic library” or “very large scale immobilized polymer synthesis” systems, the minimum size of a peptide with an activity of interest, such as binding to a CVSP16, can be determined. In addition all peptides that form the group of peptides that differ from the desired motif (or the minimum size of that motif) in one, two, or more residues can be prepared. This collection of peptides then can be screened for an ability to bind to a target molecule, e.g., a CVSP16 polypeptide or, generally, the protease domain of a CVSP16 polypeptide. This immobilized polymer synthesis system or other peptide synthesis methods also can be used to synthesize truncation analogs and deletion analogs and combinations of truncation and deletion analogs of the peptide compounds.

Peptides that bind to CVSP16 polypeptides can be prepared by classical methods known in the art, for example, by using standard solid phase techniques. The standard methods include exclusive solid phase synthesis, partial solid phase synthesis methods, fragment condensation, classical solution synthesis, and even by recombinant DNA technology (see, e.g., Merrifield (1963) J. Am. Chem. Soc., 85:2149, incorporated herein by reference.)

These procedures also can be used to synthesize peptides in which amino acids other than the 20 naturally occurring, genetically encoded amino acids are substituted at one, two, or more positions of the peptide. For instance, naphthylalanine can be substituted for tryptophan, facilitating synthesis. Other synthetic amino acids that can be substituted into the peptides include L-hydroxypropyl, L-3,4-dihydroxy-phenylalanyl, d amino acids such as L-d-hydroxylysyl and D-d-methylalanyl, L-α-methylalanyl, β amino acids, and isoquinolyl. D amino acids and non-naturally occurring synthetic amino acids also can be incorporated into the peptides (see, e.g., Roberts et al. (1983) Unusual Amino/Acids in Peptide Synthesis, 5(6):341-449).

The peptides also can be modified by phosphorylation (see, e.g., W. Bannwarth et al. (1996) Biorganic and Medicinal Chemistry Letters, 6(17):2141-2146), and other methods for making peptide derivatives (see, e.g., Hruby et al. (1990) Biochem. J., 268(2):249-262). Thus, peptide compounds also serve as a basis to prepare peptide mimetics with similar biological activity.

Those of skill in the art recognize that a variety of techniques are available for constructing peptide mimetics with the same or similar desired biological activity as the corresponding peptide compound but with more favorable activity than the peptide with respect to solubility, stability, and susceptibility to hydrolysis and proteolysis (see, e.g., Morgan et al. (1989) An. Rep. Med. Chem., 24:243-252). Methods for preparing peptide mimetics modified at the N-terminal amino group, the C-terminal carboxyl group, and/or changing one or more of the amido linkages in the peptide to a non-amido linkage are known to those of skill in the art.

Amino terminus modifications include, but are not limited to, alkylating, acetylating and adding a carbobenzoyl group, forming a succinimide group (see, e.g., Murray et al. (1995) Burger's Medicinal Chemistry and Drug Discovery, 5th ed., Vol. 1, Manfred E. Wolf, ed., John Wiley and Sons, Inc.). C-terminal modifications include mimetics wherein the C-terminal carboxyl group is replaced by an ester, an amide or modifications to form a cyclic peptide.

In addition to N-terminal and C-terminal modifications, the peptide compounds, including peptide mimetics, advantageously can be modified with or covalently coupled to one or more of a variety of hydrophilic polymers. It has been found that when peptide compounds are derivatized with a hydrophilic polymer, their solubility and circulation half-lives can be increased and their immunogenicity is masked, with little, if any, diminishment in their binding activity. Suitable nonproteinaceous polymers include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives. Generally, such hydrophilic polymers have an average molecular weight ranging from about 500 to about 100,000 daltons, including from about 2,000 to about 40,000 daltons and, from about 5,000 to about 20,000 daltons. The hydrophilic polymers can have average molecular weights of about 5,000 daltons, 10,000 daltons and 20,000 daltons. The peptide compounds can be dimerized and each of the dimeric subunits can be covalently attached to a hydrophilic polymer. The peptide compounds can be PEGylated, i.e., covalently attached to polyethylene glycol (PEG).

Methods for derivatizing peptide compounds or for coupling peptides to such polymers have been described (see, e.g., Zallipsky (1995) Bioconjugate Chem., 6:150-165; Monfardini et al. (1995) Bioconjugate Chem., 6:62-69; U.S. Pat. No. 4,640,835; U.S. Pat. No. 4,496,689; U.S. Pat. No. 4,301,144; U.S. Pat. No. 4,670,417; U.S. Pat. No. 4,791,192; U.S. Pat. No. 4,179,337 and WO 95/34326, all of which are incorporated by reference in their entirety herein).

Other methods for making peptide derivatives are described, for example, in Hruby et al. (1990), Biochem J., 268(2):249-262, which is incorporated herein by reference. Thus, the peptide compounds also serve as structural models for non-peptidic compounds with similar biological activity. Those of skill in the art recognize that a variety of techniques are available for constructing compounds with the same or similar desired biological activity as a particular peptide compound but with more favorable activity with respect to solubility, stability, and susceptibility to hydrolysis and proteolysis (see, e.g., Morgan et al. (1989) An. Rep. Med. Chem., 24:243-252, incorporated herein by reference). These techniques include replacing the peptide backbone with a backbone composed of phosphonates, amidates, carbamates, sulfonamides, secondary amines, and N-methylamino acids.

Peptide compounds can exist in a cyclized form with an intramolecular disulfide bond between the thiol groups of the cysteines. Alternatively, an intermolecular disulfide bond between the thiol groups of the cysteines can be produced to yield a dimeric (or higher oligomeric) compound. One or more of the cysteine residues also can be substituted with a homocysteine.

I. Conjugates

A conjugate, containing: a) a single chain protease domain (or proteolytically active portion thereof) of a CVSP16 polypeptide or a full length zymogen, activated form thereof, or two or single chain protease domain thereof; and b) a targeting agent linked to the CVSP16 polypeptide directly or via a linker, wherein the agent facilitates: i) affinity isolation or purification of the conjugate; ii) attachment of the conjugate to a surface; iii) detection of the conjugate; or iv) targeted delivery to a selected tissue or cell, is provided herein. The conjugate can be a chemical conjugate or a fusion protein mixture thereof.

The targeting agent can be a protein or peptide fragment, such as a tissue specific or tumor specific monoclonal antibody or growth factor or fragment thereof linked either directly or via a linker to a CVSP16 polypeptide or a protease domain thereof. The targeting agent also can be a protein or peptide fragment that contains a protein binding sequence, a nucleic acid binding sequence, a lipid binding sequence, a polysaccharide binding sequence, or a metal binding sequence, or a linker for attachment to a solid support. In a particular embodiment, the conjugate contains a) the CVSP16 or portion thereof, as described herein; and b) a targeting agent linked to the CVSP16 polypeptide directly or via a linker.

Conjugates, such as fusion proteins and chemical conjugates, of the CVSP16 polypeptide with a protein or peptide fragment (or plurality thereof) that functions, for example, to facilitate affinity isolation or purification of the CVSP16 polypeptide domain, attachment of the CVSP16 polypeptide domain to a surface, or detection of the CVSP16 polypeptide domain are provided. The conjugates can be produced by chemical conjugation, such as via thiol linkages, and can be produced by recombinant means as fusion proteins. In the fusion protein, the peptide or fragment thereof is linked to either the N-terminus or C-terminus of the CVSP16 polypeptide domain. In chemical conjugates the peptide or fragment thereof can be linked anywhere that conjugation can be effected, and there can be a plurality of such peptides or fragments linked to a single CVSP16 polypeptide domain or to a plurality thereof.

The targeting agent is for in vitro or in vivo delivery to a cell or tissue, and includes agents such as cell or tissue-specific antibodies, growth factors and other factors that bind to moieties expressed on specific cells; and other cell or tissue specific agents that promote directed delivery of a linked protein. The targeting agent can be one that specifically delivers the CVSP16 polypeptide to selected cells by interaction with a cell surface protein and internalization of conjugate or CVSP16 polypeptide portion thereof.

These conjugates are used in a variety of methods and are particularly suited for use in methods of activation of prodrugs, such as prodrugs that upon cleavage by the particular CVSP16, which is localized at or near the targeted cell or tissue, are cytotoxic. The prodrugs are administered prior to, or simultaneously with, or subsequently to the conjugate. Upon delivery to the targeted cells, the protease activates the prodrug, which then exhibits a therapeutic effect, such as a cytotoxic effect.

1. Conjugation

Conjugates with linked CVSP16 polypeptides and/or domains thereof can be prepared either by chemical conjugation, recombinant DNA technology, or by combinations of recombinant expression and chemical conjugation. The CVSP16 polypeptide domains and the targeting agent can be linked in any orientation and more than one targeting agents and/or CVSP16 polypeptide domains can be present in a conjugate.

### a. Fusion Proteins

Fusion proteins are provided herein. A fusion protein contains: a) one or a plurality of domains of a CVSP16 polypeptide; and b) a targeting agent. The fusion proteins are generally produced by recombinant expression of nucleic acids that encode the fusion protein.

### b. Chemical Conjugation

To effect chemical conjugation herein, the CVSP16 polypeptide domain is linked via one or more selected linkers or directly to the targeting agent. Chemical conjugation must be used if the targeted agent is other than a peptide or protein, such as a nucleic acid or a non-peptide drug. Any means known to those of skill in the art for chemically conjugating selected moieties can be used.

2. Linkers

Linkers for can be included in the conjugates. The conjugates can include one or more linkers between the CVSP16 polypeptide portion and the targeting agent. Additionally, linkers are used for facilitating or enhancing immobilization of a CVSP16 polypeptide or portion thereof on a solid support, such as a microtiter plate, silicon or silicon-coated chip, glass or plastic support, such as for high throughput solid phase screening protocols. Any linker known to those of skill in the art for preparation of conjugates can be used herein. These linkers are typically used in the preparation of chemical conjugates; peptide linkers can be incorporated into fusion proteins.

Linkers can be any moiety suitable to associate a domain of CVSP16 polypeptide and a targeting agent. Such linkers and linkages include, but are not limited to, peptidic linkages, amino acid and peptide linkages, typically containing between one and about 60 amino acids, more generally between about 10 and 30 amino acids, chemical linkers, such as heterobifunctional cleavable cross-linkers, including but are not limited to, N-succinimidyl (4-iodoacetyl)-aminobenzoate, sulfosuccinimidyl (4-iodoacetyl)-aminobenzoate, 4-succinimidyl-oxycarbonyl-α-(2-pyridyldithio)-toluene, sulfosuccinimidyl-6-[α-methyl-α-(pyridyldithiol)-toluamido]hexanoate, N-succinimidyl-3-(-2-pyridyldithio)-propionate, succinimidyl 6[3(-(-2-pyridyidithio)-propionamido]hexanoate, sulfosuccinimidyl 6[3(-(-2-pyridyldithio)-propionamido]hexanoate, 3-(2-pyridyidithio)-propionyl hydrazide, Ellman's reagent, dichlorotriazinic acid, and S-(2-thiopyridyl)-L-cysteine. Other linkers include, but are not limited to peptides and other moieties that reduce steric hindrance between the domain of CVSP16 polypeptide and the targeting agent, intracellular enzyme substrates, linkers that increase the flexibility of the conjugate, linkers that increase the solubility of the conjugate, linkers that increase the serum stability of the conjugate, photocleavable linkers and acid cleavable linkers.

Other exemplary linkers and linkages that are suitable for chemically linked conjugates include, but are not limited to, disulfide bonds, thioether bonds, hindered disulfide bonds, and covalent bonds between free reactive groups, such as amine and thiol groups. These bonds are produced using heterobifunctional reagents to produce reactive thiol groups on one or both of the polypeptides and then reacting the thiol groups on one polypeptide with reactive thiol groups or amine groups to which reactive maleimido groups or thiol groups can be attached on the other. Other linkers include, acid cleavable linkers, such as bismaleimideothoxy propane, acid labile-transferrin conjugates and adipic acid diihydrazide, that would be cleaved in more acidic intracellular compartments; cross linkers that are cleaved upon exposure to UV or visible light and linkers, such as the various domains, such as C_{H}1, C_{H}2, and C_{H}3, from the constant region of human IgG₁ (see, Batra et al. Molecular Immunol. 30:379-386 (1993)). In some embodiments, several linkers can be included in order to take advantage of desired properties of each linker.

Chemical linkers and peptide linkers can be inserted by covalently coupling the linker to the domain of CVSP16 polypeptide and the targeting agent. The heterobifunctional agents, described below, can be used to effect such covalent coupling. Peptide linkers also can be linked by expressing DNA encoding the linker and therapeutic agent (TA), linker and targeted agent, or linker, targeted agent and therapeutic agent (TA) as a fusion protein. Flexible linkers and linkers that increase solubility of the conjugates are contemplated for use, either alone or with other linkers are also contemplated herein.

### a) Acid Cleavable, Photocleavable and Heat Sensitive Linkers

Acid cleavable linkers, photocleavable and heat sensitive linkers also can be used, particularly where it can be necessary to cleave the domain of CVSP16 polypeptide to permit it to be more readily accessible to reaction. Acid cleavable linkers include, but are not limited to, bismaleimideothoxy propane; and adipic acid dihydrazide linkers (see, e.g., Fattom et al. (1992) Infection & Immun. 60:584-589) and acid labile transferrin conjugates that contain a sufficient portion of transferrin to permit entry into the intracellular transferrin cycling pathway (see, e.g., Welhöner et al. (1991) J. Biol. Chem. 266:4309-4314).

Photocleavable linkers are linkers that are cleaved upon exposure to light (see, e.g., Goldmacher et al. (1992) Bioconj. Chem. 3:104-107, which linkers are herein incorporated by reference), thereby releasing the targeted agent upon exposure to light. Photocleavable linkers that are cleaved upon exposure to light are known (see, e.g., Hazum et al. (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem 190:69-82, which describes water soluble photocleavable copolymers, including hydroxypropylmethacrylamide copolymer, glycine copolymer, fluorescein copolymer and methylrhodamine copolymer; Goldmacheralso et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl chloride cross linking reagents that produce photocleavable linkages), thereby releasing the targeted agent upon exposure to light. Such linkers would have particular use in treating dermatological or ophthalmic conditions that can be exposed to light using fiber optics. After administration of the conjugate, the eye or skin or other body part can be exposed to light, resulting in release of the targeted moiety from the conjugate. Such photocleavable linkers are useful in connection with diagnostic protocols in which it is desirable to remove the targeting agent to permit rapid clearance from the body of the animal.

### b) Other Linkers for Chemical Conjugation

Other linkers, include trityl linkers, particularly, derivatized trityl groups to generate a genus of conjugates that provide for release of therapeutic agents at various degrees of acidity or alkalinity. The flexibility thus afforded by the ability to preselect the pH range at which the therapeutic agent is released allows selection of a linker based on the known physiological differences between tissues in need of delivery of a therapeutic agent (see, e.g., U.S. Pat. No. 5,612,474). For example, the acidity of tumor tissues appears to be lower than that of normal tissues.

### c) Peptide Linkers

The linker moieties can be peptides. Peptide linkers can be employed in fusion proteins and also in chemically linked conjugates. The peptide typically has from about 2 to about 60 amino acid residues, for example from about 5 to about 40, or from about 10 to about 30 amino acid residues. The length selected depends upon factors, such as the use for which the linker is included.

Peptide linkers are advantageous when the targeting agent is proteinaceous. For example, the linker moiety can be a flexible spacer amino acid sequence, such as those known in single-chain antibody research. Examples of such known linker moieties include, but are not limited to, peptides, such as (GlyₘSer)ₙ and (SerₘGly)ₙ, in which n is 1 to 6, including 1 to 4 and 2 to 4, and m is 1 to 6, including 1 to 4, and 2 to 4, enzyme cleavable linkers and others.

Additional linking moieties are known. See, for example, Huston et al., Proc. Natl. Acad. Sci. U.S.A. 85:5879-5883, 1988; Whitlow, M., et al., Protein Engineering 6:989-995, 1993; Newton et al., Biochemistry 35:545-553, 1996; A. J. Cumber et al., Bioconj. Chem. 3:397-401, 1992; Ladurner et al., J. Mol. Biol. 273:330-337, 1997; and U.S. Pat. No. 4,894,443. In some embodiments, several linkers can be included in order to take advantage of desired properties of each linker.

3. Targeting Agents

Any agent that facilitates detection, immobilization, or purification of the conjugate is contemplated for use herein. For chemical conjugates any moiety that has such properties is contemplated; for fusion proteins, the targeting agent is a protein, peptide or fragment thereof that is sufficient to effect the targeting activity. Contemplated targeting agents include those that deliver the CVSP16 polypeptide or portion thereof to selected cells and tissues. Such agents include tumor specific monoclonal antibodies and portions thereof, growth factors, such as FGF, EGF, PDGF, VEGF, cytokines, including chemokines, and other such agents.

4. Nucleic Acids, Plasmids and Cells

Isolated nucleic acid fragments encoding fusion proteins are provided. The nucleic acid fragment that encodes the fusion protein includes: a) nucleic acid encoding a protease domain of a CVSP16 polypeptide; and b) nucleic acid encoding a protein, peptide or effective fragment thereof that facilitates: i) affinity isolation or purification of the fusion protein; ii) attachment of the fusion protein to a surface; or iii) detection of the fusion protein. Generally, the nucleic acid is DNA.

Plasmids for replication and vectors for expression that contain the above nucleic acid fragments are also provided. Cells containing the plasmids and vectors are also provided. The cells can be any suitable host including, but are not limited to, bacterial cells, yeast cells, fungal cells, plant cells, insect cell and animal cells. The nucleic acids, plasmids, and cells containing the plasmids can be prepared according to methods known in the art including any described herein.

Also provided are methods for producing the above fusion proteins. An exemplary method includes the steps of growing cells (i.e., culturing the cells so that they proliferate) containing a plasmid encoding the fusion protein under conditions whereby the fusion protein is expressed by the cell, and recovering the expressed fusion protein. Methods for expressing and recovering recombinant proteins are well known in the art (See generally, Current Protocols in Molecular Biology (1998) § 16, John Wiley & Sons, Inc.) and such methods can be used for expressing and recovering the expressed fusion proteins.

The recovered fusion proteins can be isolated or purified by methods known in the art such as, for example, centrifugation, filtration, chromatography, electrophoresis and immunoprecipitation, or by a combination thereof (See generally, Current Protocols in Molecular Biology (1998) § 10, John Wiley & Sons, Inc.). Generally the recovered fusion protein is isolated or purified through affinity binding between the protein or peptide fragment of the fusion protein and an affinity binding moiety. As discussed in the above sections regarding the construction of the fusion proteins, any affinity binding pairs can be constructed and used in the isolation or purification of the fusion proteins. For example, the affinity binding pairs can be protein binding sequences/protein, DNA binding sequences/DNA sequences, RNA binding sequences/RNA sequences, lipid binding sequences/lipid, polysaccharide binding sequences/polysaccharide, or metal binding sequences/metal.

5. Immobilization and Supports or Substrates Therefor

In certain embodiments, where the targeting agents are designed for linkage to surfaces, the CVSP16 polypeptide can be attached by linkage such as ionic or covalent, non-covalent or other chemical interaction, to a surface of a support or matrix material. Immobilization can be effected directly or via a linker. The CVSP16 polypeptide can be immobilized on any suitable support, including, but are not limited to, silicon chips, and other supports described herein and known to those of skill in the art. A plurality of CVSP16 polypeptide or protease domains thereof can be attached to a support, such as an array (i.e., a pattern of two or more) of conjugates on the surface of a silicon chip or other chip for use in high throughput protocols and formats.

It also is noted that the domains of the CVSP16 polypeptide can be linked directly to the surface or via a linker without a targeting agent linked thereto. Hence chips containing arrays of the domains of the CVSP16 polypeptide are also provided.

The matrix material or solid supports contemplated herein are generally any of the insoluble materials known to those of skill in the art to immobilize ligands and other molecules, and are those that are used in many chemical syntheses and separations. Such supports are used, for example, in affinity chromatography, in the immobilization of biologically active materials, and during chemical syntheses of biomolecules, including proteins, amino acids and other organic molecules and polymers. The preparation of and use of supports is well known to those of skill in this art; there are many such materials and preparations thereof known. For example, naturally-occurring support materials, such as agarose and cellulose, can be isolated from their respective sources, and processed according to known protocols, and synthetic materials can be prepared in accord with known protocols.

The supports are typically insoluble materials that are solid, porous, deformable, or hard, and have any required structure and geometry, including, but not limited to: beads, pellets, disks, capillaries, hollow fibers, needles, solid fibers, random shapes, thin films and membranes. Thus, the item can be fabricated from the matrix material or combined with it, such as by coating all or part of the surface or impregnating particles.

Typically, when the matrix is particulate, the particles are at least about 10-2000 μm, but can be smaller or larger, depending upon the selected application. Selection of the matrices is governed, at least in part, by their physical and chemical properties, such as solubility, functional groups, mechanical stability, surface area swelling propensity, hydrophobic or hydrophilic properties and intended use.

If necessary, the support matrix material can be treated to contain an appropriate reactive moiety. In some cases, the support matrix material already containing the reactive moiety can be obtained commercially. The support matrix material containing the reactive moiety can thereby serve as the matrix support upon which molecules are linked. Materials containing reactive surface moieties such as amino silane linkages, hydroxyl linkages or carboxysilane linkages can be produced by well established surface chemistry techniques involving silanization reactions, or the like. Examples of these materials are those having surface silicon oxide moieties, covalently linked to gamma-aminopropylsilane, and other organic moieties; N-[3-(triethyoxysilyl)propyl]phthalamic acid; and bis-(2-hydroxyethyl)amino-propyltriethoxysilane. Exemplary of readily available materials containing amino group reactive functionalities, include, but are not limited to, paraaminophenyltriethyoxysilane. Also derivatized polystyrenes and other such polymers are well known and readily available to those of skill in this art (e.g., the Tentagel® Resins are available with a multitude of functional groups, and are sold by Rapp Polymere, Tubingen, Germany; see, U.S. Pat. No. 4,908,405 and U.S. Pat. No. 5,292,814; see, also Butz et al. Peptide Res. 7:20-23 (1994); and Klein et al. Immunobiol. 190:53-66 (1994)).

These matrix materials include any material that can act as a support matrix for attachment of the molecules of interest. Such materials are known to those of skill in this art, and include those that are used as a support matrix. These materials include, but are not limited to, inorganics, natural polymers, and synthetic polymers, including, but are not limited to: cellulose, cellulose derivatives, acrylic resins, glass, silica gels, polystyrene, gelatin, polyvinyl pyrrolidone, co-polymers of vinyl and acrylamide, polystyrene cross-linked with divinylbenzene and others (see, Merrifield, Biochemistry 3:1385-1390 (1964)), polyacrylamides, latex gels, polystyrene, dextran, polyacrylamides, rubber, silicon, plastics, nitrocellulose, celluloses, natural sponges. Of particular interest herein, are highly porous glasses (see, e.g., U.S. Pat. No. 4,244,721) and others prepared by mixing a borosilicate, alcohol and water.

Synthetic supports include, but are not limited to: acrylamides, dextran-derivatives and dextran co-polymers, agarose-polyacrylamide blends, other polymers and co-polymers with various functional groups, methacrylate derivatives and co-polymers, polystyrene and polystyrene copolymers (see, e.g., Merrifield, Biochemistry 3:1385-1390 (1964); Berg et al. Innovation Perspect. Solid Phase Synth. Collect. Pap., Int. Symp., 1st, Epton, Roger (Ed), pp. 453-459 (1990); Berg et al., Pept., Proc. Eur. Pept. Symp., 20th, Jung, G. et al. (Eds), pp. 196-198 (1989); Berg et al., J. Am. Chem. Soc. 111:8024-8026 (1989); Kent et al., Isr. J. Chem., 17:243-247 (1979); Kent et al., J. Org. Chem. 43:2845-2852 (1978); Mitchell et al., Tetrahedron Lett. 42:3795-3798 (1976); U.S. Pat. No. 4,507,230; U.S. Pat. No. 4,006,117; and U.S. Pat. No. 5,389,449). Such materials include those made from polymers and co-polymers such as polyvinylalcohols, acrylates and acrylic acids such as poly-ethylene-co-acrylic acid, polyethylene-co-methacrylic acid, polyethylene-co-ethylacrylate, polyethylene-co-methyl acrylate, polypropylene-co-acrylic acid, polypropylene-co-methyl-acrylic acid, polypropylene-co-ethylacrylate, polypropylene-co-methyl acrylate, polyethylene-co-vinyl acetate, poly-propylene-co-vinyl acetate, and those containing acid anhydride groups such as polyethylene-co-maleic anhydride and polypropylene-co-maleic anhydride. Liposomes have also been used as solid supports for affinity purifications (Powell et al. Biotechnol. Bioeng. 33:173 (1989)).

Numerous methods have been developed for the immobilization of proteins and other biomolecules onto solid or liquid supports (see, e.g., Mosbach, Methods in Enzymology 44 (1976); Weetall, Immobilized Enzymes, Antigens, Antibodies, and Peptides, (1975); Kennedy et al., Solid Phase Biochemistry, Analytical and Synthetic Aspects, Scouten, ed., pp. 253-391 (1983); see, generally, Affinity Techniques. Enzyme Purification: Part B. Methods in Enzymology, Vol. 34, ed. W. B. Jakoby, M. Wilchek, Acad. Press, N.Y. (1974); and Immobilized Biochemicals and Affinity Chromatography, Advances in Experimental Medicine and Biology, vol. 42, ed. R. Dunlap, Plenum Press, N.Y. (1974)).

Among the most commonly used methods are absorption and adsorption or covalent binding to the support, either directly or via a linker, such as the numerous disulfide linkages, thioether bonds, hindered disulfide bonds, and covalent bonds between free reactive groups, such as amine and thiol groups, known to those of skill in art (see, e.g., the PIERCE CATALOG, ImmunoTechnology Catalog & Handbook, 1992-1993, which describes the preparation of and use of such reagents and provides a commercial source for such reagents; Wong, Chemistry of Protein Conjugation and Cross Linking, CRC Press (1993); see also DeWitt et al., Proc. Natl. Acad. Sci. U.S.A. 90:6909 (1993); Zuckermann et al., J. Am. Chem. Soc. 114:10646 (1992); Kurth et al., J. Am. Chem. Soc. 116:2661 (1994); Ellman et al., Proc. Natl. Acad. Sci. U.S.A. 91:4708 (1994); Sucholeiki, Tetrahedron Lttrs. 35:7307 (1994); Su-Sun Wang, J. Org. Chem. 41:3258 (1976); Padwa et al., J. Org. Chem. 41:3550 (1971); and Vedejs et al., J. Org. Chem. 49:575 (1984), which describe photosensitive linkers).

To effect immobilization, a composition containing the protein or other biomolecule is contacted with a support material such as alumina, carbon, an ion-exchange resin, cellulose, glass or a ceramic. Fluorocarbon polymers have been used as supports to which biomolecules have been attached by adsorption (see, U.S. Pat. No. 3,843,443; Published International PCT Application WO/86 03840).

J. Prognosis and Diagnosis

CVSP16 polypeptide proteins, domains, analogs, and derivatives thereof, and encoding nucleic acids (and sequences complementary thereto), and anti-CVSP16 polypeptide antibodies, can be used in diagnostics, particularly diagnosis of cervical cancer, colon and pancreatic cancers, and possibly other cancers, including prostate, colon, ovary, cervix and breast cancers. Such molecules can be used in assays, such as immunoassays, to detect, prognose, diagnose, or monitor various conditions, diseases, and disorders affecting CVSP16 polypeptide expression, or monitor the treatment thereof. For purposes herein, the presence of CVSP16s in body fluids or tumor tissues are of particular interest.

In particular, such an immunoassay is carried out by a method including contacting a sample derived from a patient with an anti-CVSP16 polypeptide antibody under conditions such that specific binding can occur, and detecting or measuring the amount of any specific binding by the antibody. Such binding of antibody, in tissue sections, can be used to detect aberrant CVSP16 polypeptide localization or aberrant (e.g., increased, decreased or absent) levels of CVSP16 polypeptide or aberrant activity if CVSP16 or aberrant processing of CVSP16. For example, antibody to CVSP16 polypeptide can be used to assay in a patient tissue or serum sample for the presence of CVSP16 polypeptide where an aberrant level of CVSP16 polypeptide is an indication of a diseased condition.

The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and proteinalso is A immunoassays.

CVSP16 polypeptide genes and related nucleic acid sequences and subsequences, including complementary sequences, also can be used in hybridization assays. CVSP16 polypeptide nucleic acid sequences, or subsequences thereof containing about at least 8 nucleotides, generally 14 or 16 or 30 or more, generally less than 1000 or up to 100, contiguous nucleotides can be used as hybridization probes. Hybridization assays can be used to detect, prognose, diagnose, or monitor conditions, disorders, or disease states associated with aberrant changes in CVSP16 polypeptide expression and/or activity as described herein. In particular, such a hybridization assay is carried out by a method by contacting a sample containing nucleic acid with a nucleic acid probe capable of hybridizing to CVSP16 polypeptide encoding DNA or RNA, under conditions such that hybridization can occur, and detecting or measuring any resulting hybridization.

In a specific embodiment, a method of diagnosing a disease or disorder characterized by detecting an aberrant level of a CVSP16 polypeptide in a subject is provided herein by measuring the level of the DNA, RNA, protein or activity, such as protease and/or binding activity, of a CVSP16 polypeptide in a sample derived from the subject. An increase or decrease in the level of the DNA, RNA, protein or functional activity of the CVSP16 polypeptide, relative to the level of the DNA, RNA, protein or functional activity found in an analogous sample not having the disease or disorder indicates the presence of the disease or disorder in the subject.

Kits for diagnostic use are also provided, that contain in one or more containers an anti-CVSP16 polypeptide antibody, and, optionally, a labeled binding partner to the antibody. Alternatively, the anti-CVSP16 polypeptide antibody can be labeled (with a detectable marker, e.g., a chemiluminescent, enzymatic, fluorescent, or radioactive moiety). A kit also is provided that includes in one or more containers a nucleic acid probe capable of hybridizing to SP protein-encoding RNA. In a specific embodiment, a kit can contain in one or more containers a pair of primers (e.g., each in the size range of 6-30 nucleotides) that are capable of priming amplification, e.g., by polymerase chain reaction (see e.g., Innis et al., 1990, PCR Protocols, Academic Press, Inc., San Diego, Calif.), ligase chain reaction (see EP 320,308), use of Qβ replicase, cyclic probe reaction, or other methods known in the art under appropriate reaction conditions of at least a portion of a SP protein-encoding nucleic acid. A kit can optionally further comprise in a container a predetermined amount of a purified CVSP16 polypeptide or nucleic acid, e.g., for use as a standard or control.

K. Pharmaceutical Compositions and Modes of Administration

1. Components of the Compositions

Pharmaceutical compositions containing the identified compounds that modulate the activity of a CVSP16 polypeptide are provided herein. Also provided are combinations of a compound that modulates an activity of a CVSP16 polypeptide and another treatment or compound for treatment of a neoplastic disorder, such as a chemotherapeutic compound.

The CVSP16 polypeptide modulator and the anti-tumor agent can be packaged as separate compositions for administration together or sequentially or intermittently. Alternatively, they can provided as a single composition for administration or as two compositions for administration as a single composition. The combinations can be packaged as kits.

### a. CVSP16 Polypeptide Inhibitors

Any CVSP16 polypeptide inhibitors, including those described herein when used alone or in combination with other compounds, that can alleviate, reduce, ameliorate, prevent, or place or maintain in a state of remission of clinical symptoms or diagnostic markers associated with neoplastic diseases, including undesired and/or uncontrolled angiogenesis, can be used in the present combinations.

For example, the CVSP16 polypeptide inhibitor is an antibody or fragment thereof that specifically reacts with a CVSP16 polypeptide or the protease domain thereof or other region thereof, such as the activation region, or is an inhibitor of the CVSP16 polypeptide production, an inhibitor of CVSP16 polypeptide membrane-localization or an inhibitor of the expression or activation of a CVSP16 polypeptide.

### b. Anti-Angiogenic Agents and Anti-Tumor Agents

Any anti-angiogenic agents and anti-tumor agents, including those described herein, when used alone or in combination with other compounds, that can alleviate, reduce, ameliorate, prevent, or place or maintain in a state of remission of clinical symptoms or diagnostic markers associated with undesired and/or uncontrolled angiogenesis and/or tumor growth and metastasis, particularly solid neoplasms, vascular malformations and cardiovascular disorders, chronic inflammatory diseases and aberrant wound repairs, circulatory disorders, crest syndromes, dermatological disorders, or ocular disorders, can be used in the combinations. Also contemplated are anti-tumor agents for use in combination with an inhibitor of a CVSP16 polypeptide.

### C. Anti-Tumor Agents and Anti-Angiogenic Agents

The compounds identified by the methods provided herein or provided herein can be used in combination with anti-tumor agents and/or anti-angiogenesis agents.

2. Formulations and Route of Administration

The compounds herein and agents can be formulated as pharmaceutical compositions, typically for single dosage administration. The concentrations of the compounds in the formulations are effective for delivery of an amount, upon administration, that is effective for the intended treatment. Typically, the compositions are formulated for single dosage administration. To formulate a composition, the weight fraction of a compound or mixture thereof is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds can be formulated as the sole pharmaceutically active ingredient in the composition or can be combined with other active ingredients. Liposomal suspensions, including tissue-targeted liposomes, also can be suitable as pharmaceutically acceptable carriers. These can be prepared according to methods known to those skilled in the art. For example, liposome formulations can be prepared as described in U.S. Pat. No. 4,522,811.

The active compound is included in the pharmaceutically acceptable carrier in an amount sufficient to exert a therapeutically useful effect in the absence of undesirable side effects on the patient treated. The therapeutically effective concentration can be determined empirically by testing the compounds in known in vivo and in vivo systems, such as the assays provided herein.

The concentration of active compound in the drug composition depends on absorption, inactivation and excretion rates of the active compound, the physicochemical characteristics of the compound, the dosage schedule, and amount administered as well as other factors known to those of skill in the art.

Typically a therapeutically effective dosage is contemplated. The amounts administered can be on the order of 0.001 to 1 mg/ml, including about 0.005-0.05 mg/ml and about 0.01 mg/ml, of blood volume. Pharmaceutical dosage unit forms are prepared to provide from about 1 also ismg to about 1000 mg, including from about 10 to about 500 mg, and including about 25-75 mg of the essential active ingredient or a combination of essential ingredients per dosage unit form. The precise dosage can be empirically determined.

The active ingredient can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of the claimed compositions and combinations containing them.

Pharmaceutically acceptable derivatives include acids, salts, esters, hydrates, solvates and prodrug forms. The derivative is typically selected such that its pharmacokinetic properties are superior to the corresponding neutral compound.

Thus, effective concentrations or amounts of one or more of the compounds provided herein or pharmaceutically acceptable derivatives thereof are mixed with a suitable pharmaceutical carrier or vehicle for systemic, topical or local administration to form pharmaceutical compositions. Compounds are included in an amount effective for ameliorating or treating the disorder for which treatment is contemplated. The concentration of active compound in the composition depends on absorption, inactivation, excretion rates of the active compound, the dosage schedule, amount administered, particular formulation as well as other factors known to those of skill in the art.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds can be used. Such methods are known to those of skill in this art, and include, but are not limited to, using cosolvents, such as dimethylsulfoxide (DMSO), using surfactants, such as Tween®, or dissolution in aqueous sodium bicarbonate. Derivatives of the compounds, such as prodrugs of the compounds also can be used in formulating effective pharmaceutical compositions. For ophthalmic indications, the compositions are formulated in an ophthalmically acceptable carrier. For the ophthalmic uses herein, local administration, either by topical administration or by injection are contemplated. Time release formulations are also desirable. Typically, the compositions are formulated for single dosage administration, so that a single dose administers an effective amount.

Upon mixing or addition of the compound with the vehicle, the resulting mixture can be a solution, suspension, emulsion or other composition. The form of the resulting mixture depends upon a number of factors, including the intended mode of administration and the solubility of the compound in the selected carrier or vehicle. If necessary, pharmaceutically acceptable salts or other derivatives of the compounds are prepared.

The compound is included in the pharmaceutically acceptable carrier in an amount sufficient to exert a therapeutically useful effect in the absence of undesirable side effects on the patient treated. It is understood that number and degree of side effects depends upon the condition for which the compounds are administered. For example, certain toxic and undesirable side effects are tolerated when treating life-threatening illnesses that would not be tolerated when treating disorders of lesser consequence.

The compounds also can be mixed with other active materials, that do not impair the desired action, or with materials that supplement the desired action known to those of skill in the art. The formulations of the compounds and agents for use herein include those suitable for oral, rectal, topical, inhalational, buccal (e.g., sublingual), parenteral (e.g., subcutaneous, intramuscular, intradermal, or intravenous), transdermal administration or any route. The most suitable route in any given case depends on the nature and severity of the condition being treated and on the nature of the particular active compound which is being used. The formulations are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered also can contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Methods of preparing such dosage forms are known, or will be apparent, to those skilled in this art (see, e.g., Remington's Pharmaceutical Sciences, Mack Publishing Company, Easton, Pa., 15th Edition, 1975). The composition or formulation to be administered contains a quantity of the active compound in an amount sufficient to alleviate the symptoms of the treated subject.

Dosage forms or compositions containing active ingredient in the range of 0.005% to 100% with the balance made up from non-toxic carrier can be prepared. For oral administration, the pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

The pharmaceutical preparation also can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration can be presented as unit dose suppositories. These can be prepared by admixing the active compound with one or more conventional solid carriers, for example, cocoa butter, and then shaping the resulting mixture.

Formulations suitable for topical application to the skin or to the eye generally are formulated as an ointment, cream, lotion, paste, gel, spray, aerosol and oil. Carriers which can be used include vaseline, lanoline, polyethylene glycols, alcohols, and combinations of two or more thereof. The topical formulations can further advantageously contain 0.05 to 15 percent by weight of thickeners selected from among hydroxypropyl methyl cellulose, methyl cellulose, polyvinylpyrrolidone, polyvinyl alcohol, poly (alkylene glycols), poly/hydroxyalkyl, (meth)acrylates or poly(meth)acrylamides. A topical formulation is often applied by instillation or as an ointment into the conjunctival sac. It also can be used for irrigation or lubrication of the eye, facial sinuses, and external auditory meatus. It also can be injected into the anterior eye chamber and other places. The topical formulations in the liquid state can be also present in a hydrophilic three-dimensional polymer matrix in the form of a strip, contact lens, and the like from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

The compounds can be formulated for parenteral administration by injection, e.g., by bolus injection or continuous infusion. Formulations for injection can be presented in unit dosage form, e.g., in ampules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use.

Formulations suitable for transdermal administration can be presented as discrete patches adapted to remain in intimate contact with the epidermis of the recipient for a prolonged period of time. Such patches suitably contain the active compound as an optionally buffered aqueous solution of, for example, 0.1 to 0.2 M concentration with respect to the active compound. Formulations suitable for transdermal administration also can be delivered by iontophoresis (see, e.g., Pharmaceutical Research 3 (6), 318 (1986)) and typically take the form of an optionally buffered aqueous solution of the active compound.

The pharmaceutical compositions also can be administered by controlled release means and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

Desirable blood levels can be maintained by a continuous infusion of the active agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects).

The efficacy and/or toxicity of the CVSP16 polypeptide inhibitor(s), alone or in combination with other agents also can be assessed by the methods known in the art (see, e.g., O'Reilly, Investigational New Drugs 15:5-13 (1997)).

The active compounds or pharmaceutically acceptable derivatives can be prepared with carriers that protect the compound against rapid elimination from the body, such as time release formulations or coatings.

Kits containing the compositions and/or the combinations with instructions for administration thereof are provided. The kit can further include a needle or syringe, typically packaged in sterile form, for injecting the complex, and/or a packaged alcohol pad. Instructions are optionally included for administration of the active agent by a clinician or by the patient.

Finally, the compounds or CVSP16 polypeptides or protease domains thereof or compositions containing any of the preceding agents can be packaged as articles of manufacture containing packaging material, a compound or suitable derivative thereof provided herein, which is effective for treatment of diseases or disorders contemplated herein, within the packaging material, and a label that indicates that the compound or a suitable derivative thereof is for treating the diseases or disorders contemplated herein. The label can optionally include the disorders for which the therapy is warranted.

L. Methods of Treatment

The compounds identified by the methods herein are used for treating or preventing neoplastic diseases in an animal, particularly a mammal, including a human, and are provided herein. In one embodiment, the method includes administering to a mammal an effective amount of an inhibitor of a CVSP16 polypeptide, whereby the disease or disorder is treated or prevented.

In an embodiment, the CVSP16 polypeptide inhibitor used in the treatment or prevention is administered with a pharmaceutically acceptable carrier or excipient. The mammal treated can be a human. The inhibitors provided herein are those identified by the screening assays. In addition, antibodies and antisense nucleic acids or double-stranded RNA (dsRNA), such as RNAi, are contemplated.

The treatment or prevention method can further include administering an anti-angiogenic treatment or agent or anti-tumor agent simultaneously with, prior to or subsequent to the CVSP16 polypeptide inhibitor, which can be any compound identified that inhibits the activity of a CVSP16 polypeptide. Such compounds include small molecule modulators, a natural product or derivative thereof, an antibody or a fragment or derivative thereof containing a binding region thereof against the CVSP16 polypeptide, an antisense nucleic acid or double-stranded RNA (dsRNA), such as RNAi, encoding a portion of the CVSP16 polypeptide (or the complement thereof), and a nucleic acid containing at least a portion of a gene encoding the CVSP16 polypeptide into which a heterologous nucleotide sequence has been inserted such that the heterologous sequence inactivates the biological activity of at least a portion of the gene encoding the CVSP16 polypeptide, in which the portion of the gene encoding a CVSP16 polypeptide flanks the heterologous sequence to promote homologous recombination with a genomic gene (or endogenous gene) encoding a CVSP16 polypeptide. In addition, such molecules are generally less than about 1000 nt long.

1. Antisense Treatment

In a specific embodiment, as described hereinabove, CVSP16 polypeptide function is reduced or inhibited by CVSP16 polypeptide antisense nucleic acids, to treat or prevent neoplastic disease. The therapeutic or prophylactic use of nucleic acids of at least six nucleotides that are antisense to a gene or cDNA encoding CVSP16 polypeptide or a portion thereof. A CVSP16 polypeptide “antisense” nucleic acid as used herein refers to a nucleic acid capable of hybridizing to a portion of a CVSP16 polypeptide RNA (generally mRNA) by virtue of some sequence complementarity, and generally under high stringency conditions. The antisense nucleic acid can be complementary to a coding and/or noncoding region of a CVSP16 polypeptide mRNA. Such antisense nucleic acids have utility as therapeutics that reduce or inhibit CVSP16 polypeptide function, and can be used in the treatment or prevention of disorders as described supra.

The CVSP16 polypeptide antisense nucleic acids are of at least six nucleotides and are generally oligonucleotides (ranging from 6 to about 150 nucleotides including 6 to 50 nucleotides). The antisense molecule can be complementary to all or a portion of the protease domain. For example, the oligonucleotide is at least 10 nucleotides, at least 15 nucleotides, at least 100 nucleotides, or at least 125 nucleotides. The oligonucleotides can be DNA or RNA or chimeric mixtures or derivatives or modified versions thereof, single-stranded or double-stranded. The oligonucleotide can be modified at the base moiety, sugar moiety and/or phosphate backbone. The oligonucleotide can include other appending groups such as peptides, or agents facilitating transport across the cell membrane (see, e.g., Letsinger et al., Proc. Natl. Acad. Sci. U.S.A. 86:6553-6556 (1989); Lemaitre et al., Proc. Natl. Acad. Sci. U.S.A. 84:648-652 (1987); PCT Publication No. WO 88/09810, published Dec. 15, 1988) or blood-brain barrier (see, e.g., PCT Publication No. WO 89/10134, published Apr. 25, 1988), hybridization-triggered cleavage agents (see, e.g., Krol et al., BioTechniques 6:958-976 (1988)) or intercalating agents (see, e.g., Zon, Pharm. Res. 5:539-549 (1988)).

The CVSP16 polypeptide antisense nucleic acid generally is an oligonucleotide, typically single-stranded DNA or RNA or an analog thereof or mixtures thereof. For example, the oligonucleotide includes a sequence antisense to a portion of a huma CVSP16 polypeptide. The oligonucleotide can be modified at any position on its structure with substituents generally known in the art.

The CVSP16 polypeptide antisense oligonucleotide can include at least one modified base moiety which is selected from the group including, but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide includes at least one modified sugar moiety selected from the group including but not limited to arabinose, 2-fluoroarabinose, xylulose, and hexose. The oligonucleotide can include at least one modified phosphate backbone selected from a phosphorothioate, a phosphorodithioate, a phosphoramidothioate, a phosphoramidate, a phosphordiamidate, a methylphosphonate, an alkyl phosphotriester, and a formacetal or analog thereof.

The oligonucleotide can be an α-anomeric oligonucleotide. An α-anomeric oligonucleotide forms specific double-stranded hybrids with complementary RNA in which the strands run parallel to each other (Gautier et al., Nucl. Acids Res. 15:6625-6641 (1987)).

The oligonucleotide can be conjugated to another molecule, e.g., a peptide, hybridization triggered cross-linking agent, transport agent and hybridization-triggered cleavage agent.

The oligonucleotides can be synthesized by standard methods known in the art, e.g. by use of an automated DNA synthesizer (such as are commercially available from Biosearch, Applied Biosystems, and other sources). As examples, phosphorothioate oligonucleotides can be synthesized by the method of Stein et al. (Nucl. Acids Res. 16:3209 (1988)), methylphosphonate oligonucleotides can be prepared by use of controlled pore glass polymer supports (Sarin et al., Proc. Natl. Acad. Sci. U.S.A. 85:7448-7451 (1988)), and others.

In a specific embodiment, the CVSP16 polypeptide antisense oligonucleotide includes catalytic RNA or a ribozyme (see, e.g., PCT International Publication WO 90/11364, published Oct. 4, 1990; Sarver et al., Science 247:1222-1225 (1990)). In another embodiment, the oligonucleotide is a 2′-O-methylribonucleotide (Inoue et al., Nucl. Acids Res. 15:6131-6148 (1987)), or a chimeric RNA-DNA analogue (Inoue et al., FEBS Lett. 215:327-330 (1987)). Alternatively, the oligonucleotide can be double-stranded RNA (dsRNA) such as RNAi.

In an alternative embodiment, the CVSP16 polypeptide antisense nucleic acid is produced intracellularly by transcription from an exogenous sequence. For example, a vector can be introduced in vivo such that it is taken up by a cell, within which cell the vector or a portion thereof is transcribed, producing an antisense nucleic acid (RNA). Such a vector would contain a sequence encoding the CVSP16 polypeptide antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the CVSP16 polypeptide antisense RNA can be by any promoter known in the art to act in mammalian, including human, cells. Such promoters can be inducible or constitutive. Such promoters include but are not limited to: the SV40 early promoter region (Bernoist and Chambon, Nature 290:304-310 (1981), the promoter contained in the 3′ long terminal repeat of Rous sarcoma virus (Yamamoto et al., Cell 22:787-797 (1980), the herpes thymidine kinase promoter (Wagner et al., Proc. Natl. Acad. Sci. U.S.A. 78:1441-1445 (1981), the regulatory sequences of the metallothionein gene (Brinster et al., Nature 296:39-42 (1982)).

The antisense nucleic acids include sequence complementary to at least a portion of an RNA transcript of a CVSP16 polypeptide gene, including a huma CVSP16 polypeptide gene. Absolute complementarily is not required.

The amount of CVSP16 polypeptide antisense nucleic acid that is effective in the treatment or prevention of neoplastic disease depends on the nature of the disease, and can be determined empirically by standard clinical techniques. Where possible, it is desirable to determine the antisense cytotoxicity in cells in vitro, and then in useful animal model systems prior to testing and use in humans.

2. RNA Interference

RNA interference (RNAi) (see, e.g. Chuang et al. (2000) Proc. Natl. Acad. Sci. U.S.A. 97:4985) can be employed to inhibit the expression of a gene encoding a CVSP16. Interfering RNA (RNAi) fragments, particularly double-stranded (ds) RNAi, can be used to generate loss-of-CVSP16 function. Methods relating to the use of RNAi to silence genes in organisms including, mammals, C. elegans, Drosophila and plants, and humans are known (see, e.g., Fire et al. (1998) Nature 391:806-811 Fire (1999) Trends Genet. 15:358-363; Sharp (2001) Genes Dev. 15:485-490; Hammond, et al. (2001) Nature Rev. Genet. 2:110-1119; Tuschl (2001) Chem. Biochem. 2:239-245; Hamilton et al. (1999) Science 286:950-952; Hammond et al. (2000) Nature 404:293-296; Zamore et al. (2000) Cell 101:25-33; Bernstein et al. (2001) Nature 409: 363-366; Elbashir et al. (2001) Genes Dev. 15:188-200; Elbashir et al. (2001) Nature 411:494-498; International PCT application No. WO 01/29058; International PCT application No. WO 99/32619). By selecting appropriate sequences, expression of dsRNA can interfere with accumulation of endogenous mRNA encoding a CVSP16.

Double-stranded RNA (dsRNA)-expressing constructs are introduced into a host, such as an animal or plant. This can be accomplished by any of numerous methods known in the art, for example by including it in a replicable vector, such as a viral vector (see discussion below), that remains episomal or integrates into the genome. The dsRNA can be introduced into an appropriate nucleic acid expression vector and administering it so that it becomes intracellular, e.g., by infection using a defective or attenuated retroviral or other viral vector (see U.S. Pat. No. 4,980,286). Other methods include, but are not limited to, direct injection of naked DNA, using microparticle bombardment (e.g., a gene gun; Biolistic, Dupont), coating with lipids or cell-surface receptors or transfecting agents, encapsulation in liposomes, microparticles, or microcapsules, administering it in linkage to a peptide which is known to enter the nucleus, administering it in linkage to a ligand subject to receptor-mediated endocytosis (see e.g., Wu and Wu, J. Biol. Chem. 262:4429-4432 (1987)) (which can be used to target cell types specifically expressing the receptors) and other methods. In other methods, a nucleic acid-ligand complex can be formed in which the ligand is a fusogenic viral peptide to disrupt endosomes, allowing the nucleic acid to avoid lysosomal degradation. In other methods, the nucleic acid can be targeted in vivo for cell specific uptake and expression, by targeting a specific receptor (see, e.g., Published International PCT application Nos. WO 92/06180, dated Apr. 16, 1992 (Wu et al.); WO 92/22635, dated Dec. 23, 1992 (Wilson et al.); WO92/20316, dated Nov. 26, 1992 (Findeis et al.); WO93/14188, dated Jul. 22, 1993 (Clarke et al.), WO 93/20221, dated Oct. 14, 1993 (Young)). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

RNAi can be used to inhibit expression in vitro or in vivo. Regions include at least about 21 (or 21) nucleotides that are selective (i.e. unique) for CVSP16 are used to prepare the RNAi. Smaller fragments of about 21 nucleotides can be transformed directly (i.e., in vitro or in vivo) into cells; larger RNAi dsRNA molecules are generally introduced using vectors that encode them. dsRNA molecules are at least about 21 bp long or longer, such as 50, 100, 150, 200 and longer. Methods, reagents and protocols for introducing nucleic acid molecules in to cells in vitro and in vivo are known to those of skill in the art.

3. Gene Therapy

In an exemplary embodiment, nucleic acids that include a sequence of nucleotides encoding a CVSP16 polypeptide or functional domains or derivative thereof, are administered to promote CVSP16 polypeptide function, by way of gene therapy. In this embodiment, the nucleic acid produces an encoded protein (or the nucleic acid or encoded RNA) that mediates a therapeutic effect by promoting CVSP16 polypeptide function. Any of the methods for gene therapy available in the art can be used (see, Goldspiel et al., Clinical Pharmacy 12:488-505 (1993); Wu and Wu, Biotherapy 3:87-95 (1991); Tolstoshev, An. Rev. Pharmacol. Toxicol. 32:573-596 (1993); Mulligan, Science 260:926-932 (1993); and Morgan and Anderson, An. Rev. Biochem. 62:191-217 (1993); TIBTECH 11(5):155-215 (1993).

For example, one therapeutic composition for gene therapy includes a CVSP16 polypeptide-encoding nucleic acid that is part of an expression vector that expresses a CVSP16 polypeptide or domain, fragment or chimeric protein thereof in a suitable host. In particular, such a nucleic acid has a promoter operably linked to the CVSP16 polypeptide coding region, the promoter being inducible or constitutive, and, optionally, tissue-specific. In another particular embodiment, a nucleic acid molecule is used in which the CVSP16 polypeptide coding sequences and any other desired sequences are flanked by regions that promote homologous recombination at a desired site in the genome, thus providing for intrachromosomal expression of the SP protein nucleic acid (Koller and Smithies, Proc. Natl. Acad. Sci. USA 86:8932-8935 (1989); Zijlstra et al., Nature 342:435-438 (1989)).

Delivery of the nucleic acid into a patient can be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, and it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, J. Biol. Chem. 262:4429-4432 (1987)) (which can be used to target cell types specifically expressing the receptors). In another embodiment, a nucleic acid-ligand complex can be formed in which the ligand is a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., Published International PCT application Nos. WO 92/06180 dated Apr. 16, 1992 (Wu et al.); WO 92/22635 dated Dec. 23, 1992 (Wilson et al.); WO92/20316, Nov. 26, 1992 (Findeis et al.), WO93/14188, Jul. 22, 1993 (Clarke et al.), WO 93/20221, Oct. 14, 1993 (Young)). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijistra et al., Nature 342:435-438 (1989)).

In a specific embodiment, a viral vector that contains the CVSP16 polypeptide nucleic acid is used. For example, a retroviral vector can be used (see Miller et al., Meth. Enzymol. 217:581-599 (1993)). These retroviral vectors have been modified to delete retroviral sequences that are not necessary for packaging of the viral genome and integration into host cell DNA. The CVSP16 polypeptide nucleic acid to be used in gene therapy is cloned into the vector, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); and Mastrangeli et al., J. Clin. Invest. 91:225-234 (1993). Adeno-associated virus (AAV) also is used in gene therapy (Walsh et al., Proc. Soc. Exp. Biol. Med. 204:289-300 (1993).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion and other delivery methods. Numerous techniques are known in the art for the introduction of foreign genes into cells (see e.g., Loeffler and Behr, Meth. Enzymol. 217:599-618 (1993); Cohen et al., Meth. Enzymol. 217:618-644 (1993); Cline, Pharmac. Ther. 29:69-92 (1985)) and can be used, provided that the necessary developmental and physiological functions of the recipient cells are not disrupted. The technique should provide for the stable transfer of the nucleic acid to the cell, so that the nucleic acid is expressible by the cell and generally heritable and expressible by its cell progeny.

The resulting recombinant cells can be delivered to a patient by various methods known in the art. In an embodiment, epithelial cells are injected, e.g., subcutaneously. In another embodiment, recombinant skin cells can be applied as a skin graft onto the patient. Recombinant blood cells (e.g., hematopoietic stem or progenitor cells) can be administered intravenously. The amount of cells envisioned for use depends on the desired effect, patient state and other parameters, and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Talso islymphocytes, Balso islymphocytes, monocytes, macrophages, neutrophils, eosinophils, megakaryocytes, granulocytes; various stem or progenitor cells, in particular hematopoietic stem or progenitor cells, e.g., such as stem cells obtained from bone marrow, umbilical cord blood, peripheral blood, fetal liver, and other sources thereof.

For example, a cell used for gene therapy is autologous to the patient. In an embodiment in which recombinant cells are used in gene therapy, a CVSP16 polypeptide nucleic acid is introduced into the cells such that it is expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment. Such stem cells include but are not limited to hematopoietic stem cells (HSC), stem cells of epithelial tissues such as the skin and the lining of the gut, embryonic heart muscle cells, liver stem cells (PCT Publication WO 94/08598, dated Apr. 28, 1994), and neural stem cells (Stemple and Anderson, Cell 71:973-985 (1992)).

Epithelial stem cells (ESCs) or keratinocytes can be obtained from tissues such as the skin and the lining of the gut by known procedures (Rheinwald, Meth. Cell Bio. 21A:229 (1980)). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Rheinwald, Meth. Cell Bio. 21A:229 (1980); Pittelkow and Scott, Cano Clinic Proc. 61:771 (1986)). If the ESCs are provided by a donor, a method for suppression of host versus graft reactivity (e.g., irradiation, drug or antibody administration to promote moderate immunosuppression) also can be used.

With respect to hematopoietic stem cells (HSC), any technique which provides for the isolation, propagation, and maintenance in vitro of HSC can be used in this embodiment. Techniques by which this can be accomplished include (a) the isolation and establishment of HSC cultures from bone marrow cells isolated from the future host, or a donor, or (b) the use of previously established long-term HSC cultures, which can be allogeneic or xenogeneic. Non-autologous HSC generally are used with a method of suppressing transplantation immune reactions of the future host/patient. In a particular embodiment, human bone marrow cells can be obtained from the posterior iliac crest by needle aspiration (see, e.g., Kodo et al., J. Clin. Invest. 73:1377-1384 (1984)). For example, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., J. Cell Physiol. 91:335 (1977) or Witlock-Witte culture techniques (Witlock and Witte, Proc. Natl. Acad. Sci. USA 79:3608-3612 (1982)).

In a specific embodiment, the nucleic acid to be introduced for purposes of gene therapy includes an inducible promoter operably linked to the coding region, such that expression of the nucleic acid is controllable by controlling the presence or absence of the appropriate inducer of transcription.

3. Prodrugs

A method for treating tumors is provided. The method is practiced by administering a prodrug that is cleaved at a specific site by a CVSP16 to release an active drug or a precursor that can be converted to active drug in vivo. Upon contact with a cell that expresses CVSP16 activity, the prodrug is converted into an active drug. The prodrug can be a conjugate that contains the active agent, such as an anti-tumor drug, such as a cytotoxic agent, or other therapeutic agent (TA), linked to a substrate for the targeted CVSP16, such that the drug or agent is inactive or unable to enter a cell, in the conjugate, but is activated upon cleavage. The prodrug, for example, can contain an oligopeptide, typically a relatively short, less than about 10 amino acids peptide, that is proteolytically cleaved by the targeted CVSP16. Cytotoxic agents, include, but are not limited to, alkylating agents, antiproliferative agents and tubulin binding agents. Others include, vinca drugs, mitomycins, bleomycins and taxanes.

M. Animal Models

Transgenic animal models and animals, such as rodents, including mice and rats, cows, chickens, pigs, goats, sheep, monkeys, including gorillas, and other primates, are provided herein. In particular, transgenic non-human animals that contain heterologous nucleic acid encoding a CVSP16 polypeptide or a transgenic animal in which expression of the polypeptide has been altered, such as by replacing or modifying the promoter region or other regulatory region of the endogenous gene are provided. Such an animal can by produced by promoting recombination between endogenous nucleic acid and an exogenous CVSP16 gene that could be over-expressed or mis-expressed, such as by expression under a strong promoter, via homologous or other recombination event.

Transgenic animals can be produced by introducing the nucleic acid using any known method of delivery, including, but not limited to, microinjection, lipofection and other modes of gene delivery into a germline cell or somatic cells, such as an embryonic stem cell. Typically the nucleic acid is introduced into a cell, such as an embryonic stem cell (ES), followed by injecting the ES cells into a blastocyst, and implanting the blastocyst into a foster mother, which is followed by the birth of a transgenic animal.

Generally, introduction of a heterologous nucleic acid molecule into a chromosome of the animal occurs by a recombination between the heterologous CVSP16-encoding nucleic acid and endogenous nucleic acid. The heterologous nucleic acid can be targeted to a specific chromosome.

In some instances, knockout animals can be produced. Such an animal can be initially produced by promoting homologous recombination between a CVSP16 polypeptide gene in its chromosome and an exogenous CVSP16 polypeptide gene that has been rendered biologically inactive (typically by insertion of a heterologous sequence, e.g., an antibiotic resistance gene). In one embodiment, this homologous recombination is performed by transforming embryo-derived stem (ES) cells with a vector containing the insertionally inactivated CVSP16 polypeptide gene, such that homologous recombination occurs, followed by injecting the ES cells into a blastocyst, and implanting the blastocyst into a foster mother, followed by the birth of the chimeric animal (“knockout animal”) in which a CVSP16 polypeptide gene has been inactivated (see Capecchi, Science 244:1288-1292 (1989)). The chimeric animal can be bred to produce homozygous knockout animals, which can then be used to produce additional knockout animals. Knockout animals include, but are not limited to, mice, hamsters, sheep, pigs, cattle, and other non-human mammals. For example, a knockout mouse is produced. The resulting animals can serve as models of specific diseases, such as cancers, that exhibit under-expression of a CVSP16 polypeptide. Such knockout animals can be used as animal models of such diseases e.g., to screen for or test molecules for the ability to treat or prevent such diseases or disorders.

Other types of transgenic animals also can be produced, including those that over-express the CVSP16 polypeptide. Such animals include “knock-in” animals that are animals in which the normal gene is replaced by a variant, such as a mutant, an over-expressed form, or other form. For example, one species', such as a rodent's endogenous gene can be replaced by the gene from another species, such as from a human. Animals also can be produced by non-homologous recombination into other sites in a chromosome; including animals that have a plurality of integration events.

After production of the first generation transgenic animal, a chimeric animal can be bred to produce additional animals with over-expressed or mis-expressed CVSP16 polypeptides. Such animals include, but are not limited to, mice, hamsters, sheep, pigs, cattle and other non-human mammals. The resulting animals can serve as models of specific diseases, such as cancers, that exhibit over-expression or mis-expression of a CVSP16 polypeptide. Such animals can be used as animal models of such diseases e.g., to screen for or test molecules for the ability to treat or prevent such diseases or disorders. In a specific embodiment, a mouse with over-expressed or mis-expressed CVSP16 polypeptide is produced.

The following examples are included for illustrative purposes only and are not intended to limit the scope of the invention.

### EXAMPLE 1

Identification of CVSP16

The protein sequence of the protease domain of matriptase (MTSP1; accession number AF118224) was used to search the human HTGS (High Throughput Genomic Sequence) database using the blastn algorithm (www.ncbi.nlm.nih.gov/BLAST). This search and alignment algorithm compares a protein query sequence against a nucleotide sequence database dynamically translated in all six reading frames (both strands). Among the proteases identified was the protease designated herein as CVSP16. The partial protein sequence of the CVSP16 protease domain in the database shares 34% identity to the protease domain of matriptase. A search using the algorithm blastp (www.ncbi.nlm.nih.gov/BLAST) indicated that the translated sequence of CVSP16 showed 34% identity to prostamin (BAB20376.1), 36% identity to corin (NP_{—}006578.1), 36% identity to marapsin (NP_{—}114154.1), 35% identity to prostasin (NP_{—}002764.1), 39% identity to transmembrane tryptase (NP_{—}036599.1) and 36% identity to serine protease 22 (NP_{—}071402.1). Based on the incomplete and unordered human genome sequence (www.ncbi.nlm.nih.gov/genome/seq), CVSP16 appears to be localized on chromosome 16 (locus: 16p13.3; clone accession number AC009088.7). A search of sequences deposited in GenBank showed that one entry (XM_{—}097026.1) corresponding to a hypothetical, genomic sequence-derived protein had homologous nucleotide sequence (74%) with CVSP16, although the reported translated protein sequence only had 10% homology to the partial CVSP16 polypeptide sequence. A search of the EST database showed the existence of two EST clones (AW450407 from human colon and Al190509 from human fetal heart). Both EST clones shared 98-99% identical sequence to a portion of the CVSP16 protease domain sequence.

Cloning of CVSP16 from Human Small Intestine Cell Line Using RACE Reactions

Using the EST-derived cDNA sequence homologous to CVSP16, four oligonucleotide primers hybridizing within the protease domain sequence were designed and synthesized. The sequence for the 5′ end primer was 5′-CCCTCTGGGTAGCCAGCACACAGCATC-3′ SEQ ID No. 7 and that of the 3′ end primer was 5′-GCCATCGTGGTGCCGGCCAACTACAG-3′ SEQ ID No. 8. The sequence for the nested 5′ end primer was 5′-GCACACAGCATCCCTGGCAATATCTGG-3′ SEQ ID No. 9 and that of the nested 3′end primer was 5′-CGGCCAACTACAGCCAAGTGGAGCTG-3′ SEQ ID No. 10.

The first set of RACE primers were used to amplify cDNA fragments from human small intestine Marathon-ready cDNA library (catalog number 7426-1; www.clontech.com). Following this, nested RACE reactions were performed using the nested primers. Several DNA bands were detected in all RACE reactions. The pool of cDNA fragments larger than 500 bp was isolated by 2% agarose electrophoresis and purified from the nested 5′- and 3′-RACE reactions using the MinElute gel extraction kit (catalog number 28606; www.qiagen.com), then subcloned into an E. coli vector (pCR2.1TOPO; catalog no. K-4500-01; www.invitrogen.com) and transformed into E. coli TOP10 cells (www.invitrogen.com). To identify clones that contained CVSP16 cDNA, colony hybridization was performed using a ˜300-bp cDNA probe amplified from the nested 5′- and 3′-RACE primers on the same small intestine Marathon cDNA library. Subsequent sequence analysis confirmed that the nucleotide sequence of these 5′- and 3′-RACE-derived clones matched that of the CVSP16 sequence using a fluorescent dye-based DNA sequencing method (catalog number 4390244; ABI PRISM® BigDye™ Terminator v 3.0 Ready Reaction Cycle Sequencing Kits with AmpliTaq® DNA Polymerase, FS; home.appliedbiosystems.com). A methionine start codon was missing from the cDNA clone, but an in-frame stop codon could be found in the coding sequence; thus, the 5′-RACE product did not extend to the beginning of the coding sequence.

PCR Amplification of the Full-Length cDNA Encoding CVSP16

A search of the Incyte proprietary database showed a clone (228456.2) that had homology to the CVSP16 partial sequence. Based on this sequence, several primer sets were designed and synthesized. To obtain the full-length CVSP16 cDNA, 2 pairs of gene-specific primers that amplify 2 overlapping portions of the CVSP16 sequence and the cDNA library made from human liver were used. The first pair of primers used was:
5′-ATGGCCCGGCAGCTGCTCCTCCCCCTTGTG-3′ SEQ ID No. 11 for the 5′ end (putative start codon underlined) and
5′-CGGCTCCCGGGCAGGAAGTAGTGTTCCG-3′ SEQ ID No. 12 for the 3′ end. This primer pair amplified the initial half of the CVSP16 sequence.
The second pair of primers used was: 5′-TGGGTCTTGGCACCTGCCAGCTGCTTTCTG-3′ SEQ ID No. 15 at the 5′ end and
5′-GAAGGGGGAAGTGGTGCTGGGACCCTAG-3′ SEQ ID No. 16 for the 3′ end. This pair amplified the last half of the CVSP16 sequence and the 3′-end primer corresponds to the sequence downstream of the putative stop codon. Two cDNA fragments (˜1.3 and ˜1.4 kbp) were amplified using these 2 sets of primers. The PCR products were isolated by 2% agarose electrophoresis, purified using the MinElute gel extraction kit (www.qiagen.com) and subcloned into pCR2.1TOPO (www.invitrogen.com). Sequence analysis was performed to confirm the nucleotide sequence.

The full-length coding region of CVSP16 was prepared by stitching the two cDNA fragments using PCR. A ˜2.3 kbp fragment was amplified, isolated by 1% agarose electrophoresis, purified, subcloned and sequenced as described above. The sequence obtained from this gene-specific amplification of CVSP16 matched those sequences obtained from both 5′- and 3′-RACE reactions. In addition the missing 5′ end containing a start codon was present.

Gene Expression Profile of CVSP16 in Normal, Tumor Tissues and Cell Lines

To obtain information regarding the gene expression profile of the CVSP16 transcript, a ˜370-bp CVSP16 cDNA fragment was used to probe a dot blot composed of polyA⁺RNAs extracted and purified from 76 different human tissues (Human Multiple Tissue Expression (MTE) Array; catalog no. 7775-1; www.clontech.com). The cDNA probe was amplified from human small intestine Marathon cDNA library using the following primers: 5′ end primer, 5′-CCCTCTGGGTAGCCAGCACACAGCATC-3′ SEQ ID No. 17, and 3′ end primer, 5′-GCCATCGTGGTGCCGGCCAACTACAG-3′ SEQ ID No. 18. The results indicate that the CVSP16 transcript is strongly expressed in several tissues including kidney, stomach, colon, spleen, thyroid gland, trachea and pituitary gland. The CVSP16 transcript also is found in many other tissues albeit at a lower level. Among tumor cell lines, the CVSP16 transcript is found (in decreasing signal intensity) in cervical Hela S3, lung A549, leukemia K-562, Burkitt's lymphoma Raji, leukemia HL-60, colorectal SW480, Burkitt's lymphoma Daudi and leukemia MOLT-4. To compare the expression profile of CVSP16 transcript in a range of normal human and matched tumor tissues, a matched tumor/normal expression array (catalog number 7840-1; www.clontech.com) composed of 68 paired cDNA samples from individual patients was used. Results show that the CVSP16 transcript is expressed at a low level in a number of normal tissues including breast, prostate, cervix, uterus, colon, lung, small intestine, stomach, kidney and rectum, but is not differentially expressed in any of the matched tumors. Several SMART™ 5′-RACE cDNA libraries (catalog number K 1811-1; www.clontech.com) prepared from normal breast, normal testes, normal prostate, prostate cancer cell lines and breast cancer cell lines were analyzed for the presence of CVSP16 transcript by RT-PCR using gene-specific primers. The primer sequences were: 5′-ATCGTGGTGCCGGCCAACTACAGCCAAGTG-3′ SEQ ID No. 19 for the 5′ end primer and 5′-ACCCATCACCTGCTCCCGTATCCATGCCTC-3′ SEQ ID No. 20 for the 3′ end primer. The CVSP16 transcript was detected (in decreasing signal intensity) in normal breast, normal prostate, breast carcinoma cell line DU4475, prostate carcinoma cell line PC-3, prostate carcinoma cell line LNCaP, breast carcinoma cell line MDA-MB-231, and breast carcinoma cell line MDA-MB-453.

Structural Features

The CVSP16 contains a signal peptide sequence (aa 1 to aa 23) and a trypsin-like serine protease domain designated herein as CVSP16 PD1 (aa 46 to aa 286) characterized by the presence of a protease activation cleavage site ( . . . R₄₆↓I₄₇VGGSNAQP . . . , where ↓ indicates protease activation cleavage site) at the beginning of the domain and the catalytic triad residues (H₈₇, D₁₃₉ and S₂₄₃) in 3 highly-conserved regions of the catalytic domain. In addition, CVSP16 has an additional 465-amino acid sequence (aa 287 to aa 752) beyond the protease domain. Analysis of this 465-amino acid long region indicates the presence of a second protease domain (aa 323 to aa 550, designated herein as CVSP16 PD2). In this domain, however, the invariant catalytic histidine is replaced by a serine (S₃₆₃) residue, and the highly conserved SGGP sequence that contains the catalytic serine has been replaced with the sequence S₅₁₀RWS. The starting residue is unusual, suggesting that cleavage may not be needed for activation. These sequences and differences from other protease domains indicate that the second protease domain has lower catalytic activity.

CVSP16 has 8 putative N-linked glycosylation sites ( . . . N₉₂GT . . . , . . . N₁₃₀YS . . . , . . . N₂₁₇LT . . . , . . . N₃₁₇CT . . . , . . . N₃₆₉SS . . . , . . . N₄₀₂AS . . . , . . . N₄₂₁LS . . . , . . . N₅₀₈DS . . . ). The following cysteine pairings are noted: C₇₂-C₈₈, C₁₇₃-C₂₄₉, C₂₀₆-C₂₂₈, C₂₃₉-C₂₆₇, C₃₄₈-C₃₆₄, C₄₄₄-C₅₁₆, C₄₇₂-C₄₉₄ and C₅₀₆-C₅₃₄. In addition, an unpaired cysteine (C₁₅₉) in the first protease domain should pair with C₃₈. An unpaired cysteine (C₄₃₀) in the second protease domain should pair with C₃₂₅ or, less likely, C₃₁₈. PD1 and PD2 have an additional Cys (C₂₀₈ and C₄₇₄, respectively) that is unpaired or pairs with a Cys outside of each protease domain. The protein has a C-terminal domain beyond 550.

Homology of CVSP16 to Other Serine Proteases

Clustal W alignment (using MacVector; version 6.5.3; www.accel-rys.com/products/macvector/index.html) of the derived CVSP16 full-length cDNA and protein sequences with those of the cDNA and protein sequences derived from the Incyte clone (228456.2) showed an 81% and 86% sequence identity, respectively. Alignment (blastp; www.ncbi.nlm.nih.gov/BLAST) of the protease domain (minus the 465-amino acid extension at the C terminus) sequence of CVSP16 shows 40% identity with that of human matriptase/MTSP-1 (accession number NP_{—}068813), 40% identity with that of human prostamin (accession number BAB20376.1), 40% identity with that of human marapsin (accession number NP_{—}114154.1), 39% identity with that of human enterokinase (accession number NP_{—}002763.1), 40% identity with that of human prostasin (accession number NP_{—}002764.1), 39% identity with that of human corin (accession number NP_{—}006587.1), 44% identity with that of human transmembrane tryptase (accession number NP_{—}036599.1), and 37% identity with that of human plasma kallikrein (accession number NP_{—}000883.1).

The CVSP16 and encoding nucleic acid has homology to a clones described in International PCT application No. WO 02/000860 (see SEQ ID No. 111 therein) and to clones in International PCT application No. WO 02/046383, International PCT application No. WO 01075067 and EP 1130094. The clones and predicted encoded polypeptides described in the PCT applications and EP application, however, differ from the nucleic acid molecule encoding CVSP16 polypeptides and the CVSP16 polypeptides provided herein. For example, each of the nucleic acid molecules described in International PCT application No. WO 02/00860 includes a sequence of nucleotides encoding the sequence of amino acids set forth in SEQ ID 21 herein, and the disclosed polypeptides include the sequence of amino acids set forth in SEQ ID 21 herein. None of the polypeptides provided herein include at least 5, 10, 15, 20 or more contiguous amino acids from SEQ ID No. 21, particularly between Gin 660 and Met 661 (SEQ ID No. 6) or between the corresponding amino acids in other CVSP16s. Hence the CVSP16s provided herein include the sequence Gln₆₆₀Met₆₆₁, particularly, the contiguous sequence Gly His Gin Met Thr Ser (see, SEQ ID No. 6, amino acids 658-663). or Leu Pro Gin Gly His Gin Met Thr Ser Ala (see, SEQ ID No. 6, amino acids 655-664).

Sequence Analysis

CVSP16 cDNA and protein sequences were analyzed using MacVector nucleic acid/protein sequence analysis program. The full length cDNA encoding CVSP16 is 2,293 bp long containing a 2,259-bp open reading frame, which translates to a 752-amino acid protein. The cDNA encoding the active protease domain is 717 bp long which translates to a 239-amino acid domain. The G+C content of the CVSP16 cDNA is 68%. Attached are the cDNA sequence and the translated protein sequence of CVSP16 (see, also, SEQ ID Nos. 5 and 6).

CVSP16 Full Length cDNA and Translated Protein Sequence

Sequence Range: 1 to 2293

```
                  10 20 30 40 50 60
ATGGCCCGGCAGCTGCTCCTCCCCCTTGTGGTGCTTGTCATCAGTCCCATCCCAGGAGCC
TACCGGGCCGTCGACGAGGAGGGGGAACACCACGAACAGTAGTCAGGGTAGGGTCCTCGG
  M A R Q L L L P L V V L V I S P I P G A>
              70 80 90 100 110 120
TTCCAGGACTCAGCTCTCAGTCCTACCCAGGAAGAACCTGAAGATCTGGACTGCGGGCGC
AAGGTCCTGAGTCGAGAGTCAGGATGGGTCCTTCTTGGACTTCTAGACCTGACGCCCGCG
  F Q D S A L S P T Q E E P E D L D C G R>
              130 140 150 160 170 180
CCTGAGCCCTCGGCCCGCATCGTGGGGGGCTCAAACGCGCAGCCGGGCACCTGGCCTTGG
GGACTCGGGAGCCGGGCGTAGCACCCCCCGAGTTTGCGCGTCGGCCCGTGGACCGGAACC
  P E P S A R I V G G S N A Q P G T W P W>
              190 200 210 220 230 240
CAAGTGAGCCTGCACCATGGAGGTGGCCACATCTGCGGGGGCTCCCTCATCGCCCCCTCC
GTTCACTCGGACGTGGTACCTCCACCGGTGTAGACGCCCCCGAGGGAGTAGCGGGGGAGG
  Q V S L H H G G G H I C G G S L I A P S>
              250 260 270 280 290 300
TGGGTCCTCTCCGCCGCTCACTGTTTCATGACGAATGGGACGCTGGAGCCCGCGGCCGAG
ACCCAGGAGAGGCGGCGAGTGACAAAGTACTGCTTACCCTGCGACCTCGGGCGCCGGCTC
  W V L S A A H C F M T N G T L E P A A E>
              310 320 330 340 350 360
TGGTCGGTACTGCTGGGCGTGCACTCCCAGGACGGGCCCCTGGACGGCGCGCACACCCGC
ACCAGCCATGACGACCCGCACGTGAGGGTCCTGCCCGGGGACCTGCCGCGCGTGTGGGCG
  W S V L L G V H S Q D G P L D G A H T R>
              370 380 390 400 410 420
GCAGTGGCCGCCATCGTGGTGCCGGCCAACTACAGCCAAGTGGAGCTGGGCGCCGACCTG
CGTCACCGGCGGTAGCACCACGGCCGGTTGATGTCGGTTCACCTCGACCCGCGGCTGGAC
  A V A A I V V P A N Y S Q V E L G A D L>
              430 440 450 460 470 480
GCCCTGCTGCGCCTGGCCTCACCCGCCAGCCTGGGCCCCGCCGTGTGGCCTGTCTGCCTG
CGGGACGACGCGGACCGGAGTGGGCGGTCGGACCCGGGGCGGCACACCGGACAGACGGAC
  A L L R L A S P A S L G P A V W P V C L>
              490 500 510 520 530 540
CCCCGCGCCTCACACCGCTTCGTGCACGGCACCGCCTGCTGGGCCACCGGCTGGGGAGAC
GGGGCGCGGAGTGTGGCGAAGCACGTGCCGTGGCGGACGACCCGGTGGCCGACCCCTCTG
  P R A S H R F V H G T A C W A T G W G D>
              550 560 570 580 590 600
GTCCAGGAGGCAGATCCTCTGCCTCTCCCCTGGGTGCTACAGGAAGTGGAGCTAAGGCTG
CAGGTCCTCCGTCTAGGAGACGGAGAGCGGACCCACGATGTCCTTCACCTCGATTCCGAC
  V Q E A D P L P L P W V L Q E V E L R L>
              610 620 630 640 650 660
CTGGGCGAGGCCACCTGTCAATGTCTCTACAGCCAGCCCGGTCCCTTCAACCTCACTCTC
GACCCGCTCCGGTGGACAGTTACAGAGATGTCGGTCGGGCCAGGGAAGTTGGAGTGAGAG
  L G E A T C Q C L Y S Q P G P F N L T L>
              670 680 690 700 710 720
CAGATATTGCCAGGGATGCTGTGTGCTGGCTACCCAGGGGGCCGCAGGGACACCTGCCAG
GTCTATAACGGTCCCTACGACACACGACCGATGGGTCCCCCGGCGTCCCTGTGGACGGTC
  Q I L P G M L C A G Y P G G R R D T C Q>
              730 740 750 760 770 780
GGTGACTCTGGGGGGCCCCTGGTCTGTGAGGAAGGCGGCCGCTGGTTCCAGGCAGGAATC
CCACTGAGACCCCCCGGGGACCAGACACTCCTTCCGCCGGCGACCAAGGTCCGTCCTTAG
  G D S G G P L V C E E G G R W F Q A G I>
              790 800 810 820 830 840
ACCAGCTTTGGCTTTGGCTGTGGACGGAGAAACCGCCCTGGAGTTTTCACTGCTGTGGCT
TGGTCGAAACCGAAACCGACACCTGCCTCTTTGGCGGGACCTCAAAAGTGACGACACCGA
  T S F G F G C G R R N R P G V F T A V A>
              850 860 870 880 890 900
ACCTATGAGGCATGGATACGGGAGCAGGTGATGGGTTCAGAGCCTGGGCCTGCCTTTCCC
TGGATACTCCGTACCTATGCCCTCGTCCACTACCCAAGTCTCGGACCCGGACGGAAAGGG
  T Y E A W I R E Q V M G S E P G P A F P>
              910 920 930 940 950 960
ACCCAGCCCCAGAAGACCCAGTCAGATCCCCAGGAGCCCAGGGAGGAGAACTGCACCATT
TGGGTCGGGGTCTTCTGGGTCAGTCTAGGGGTCCTCGGGTCCCTCCTCTTGACGTGGTAA
  T Q P Q K T Q S D P Q E P R E E N C T I>
              970 980 990 1000 1010 1020
GCCCTGCCTGAGTGCGGGAAGGCCCCGCGGCCAGGGGCCTGGCCCTGGGAGGCCCAGGTG
CGGGACGGACTCACGCCCTTCCGGGGCGCCGGTCCCCGGACCGGGACCCTCCGGGTCCAC
  A L P E C G K A P R P G A W P W E A Q V>
            1030 1040 1050 1060 1070 1080
ATGGTGCCAGGATCCAGACCCTGCCATGGGGCGCTGGTGTCTGAAAGCTGGGTCTTGGCA
TACCACGGTCCTAGGTCTGGGACGGTACCCCGCGACCACAGACTTTCGACCCAGAACCGT
  M V P G S R P C H G A L V S E S W V L A>
            1090 1100 1110 1120 1130 1140
CCTGCCAGCTGCTTTCTGGACCCGAACAGCTCCGACAGCCCACCCCGCGACCTCGACGCC
GGACGGTCGACGAAAGACCTGGGCTTGTCGAGGCTGTCGGGTGGGGCGCTGGAGCTGCGG
  P A S C F L D P N S S D S P P R D L D A>
            1150 1160 1170 1180 1190 1200
TGGCGCGTGCTGCTGCCCTCGCACCCGCGCGCGGAGCGGGTGGCGCGCCTGGTGCAGCAC
ACCGCGCACGACGACGGGAGCGTGGGCGCGCGCCTCGCCCACCGCGCGGACCACGTCGTG
  W R V L L P S H P R A E R V A R L V Q H>
            1210 1220 1230 1240 1250 1260
GAGAACGCTTCGTGGGACAACGCCCCGGACCTGGCGCTGCTGCAGCTGCGCACGCCCGTG
CTCTTGCGAAGCACCCTGTTGCGGGGCCTGGACCGCGACGACGTCGACGCGTGCGGGCAC
  E N A S W D N A P D L A L L Q L R T P V>
            1270 1280 1290 1300 1310 1320
AACCTGAGTGCGGCTTCGCGGCCCGTGTGCCTACCCCACCCGGAACACTACTTCCTGCCC
TTGGACTCACGCCGAAGCGCCGGGCACACGGATGGGGTGGGCCTTGTGATGAAGGACGGG
  N L S A A S R P V C L P H P E H Y F L P>
            1330 1340 1350 1360 1370 1380
GGGAGCCGCTGCCGCCTGGCCCGCTGGGGCCGCGGGGAACCCGCGCTTGGCCCAGGCGCG
CCCTCGGCGACGGCGGACCGGGCGACCCCGGCGCCCCTTGGGCGCGAACCGGGTCCGCGC
  G S R C R L A R W G R G E P A L G P G A>
            1390 1400 1410 1420 1430 1440
CTGCTGGAGGCGGAGCTGTTAGGCGGCTGGTGGTGCCACTGCCTGTACGGCCGCCAGGGG
GACGACCTCCGCCTCGACAATCCGCCGACCACCACGGTGACGGACATGCCGGCGGTCCCC
  L L E A E L L G G W W C H C L Y G R Q G>
            1450 1460 1470 1480 1490 1500
GCGGCAGTACCGCTGCCCGGAGACCCGCCGCACGCGCTCTGCCCTGCCTACCAGGAAAAG
CGCCGTCATGGCGACGGGCCTCTGGGCGGCGTGCGCGAGACGGGACGGATGGTCCTTTTC
  A A V P L P G D P P H A L C P A Y Q E K>
            1510 1520 1530 1540 1550 1560
GAGGAGGTGGGCAGCTGCTGGAATGACTCGCGTTGGAGCCTTTTGTGCCAGGAGGAGGGG
CTCCTCCACCCGTCGACGACCTTACTGAGCGCAACCTCGGAAAACACGGTCCTCCTCCCC
  E E V G S C W N D S R W S L L C Q E E G>
            1570 1580 1590 1600 1610 1620
ACCTGGTTTCTGGCTGGAATCAGAGACTTTCCCAGTGGCTGTCTACGTCCCCGAGCCTTC
TGGACCAAAGACCGACCTTAGTCTCTGAAAGGGTCACCGACAGATGCAGGGGCTCGGAAG
  T W F L A G I R D F P S G C L R P R A F>
            1630 1640 1650 1660 1670 1680
TTCCCTCTGCAGACTCATGGCCCATGGATCAGCCATGTGACTCGGGGAGCCTACCTGGAG
AAGGGAGACGTCTGAGTACCGGGTACCTAGTCGGTACACTGAGCCCCTCGGATGGACCTC
  F P L Q T H G P W I S H V T R G A Y L E>
            1690 1700 1710 1720 1730 1740
GACCAGCTAGCCTGGGACTGGGGCCCTGATGGGGAGGAGACTGAGACACAGACTTGTCCC
CTGGTCGATCGGACCCTGACCCCGGGACTACCCCTCCTCTGACTCTGTGTCTGAACAGGG
  D Q L A W D W G P D G E E T E T Q T C P>
            1750 1760 1770 1780 1790 1800
CCACACACAGAGCATGGTGCCTGTGGCCTGCGGCTGGAGGCTGCTCCAGTGGGGGTCCTG
GGTGTGTGTCTCGTACCACGGACACCGGACGCCGACCTCCGACGAGGTCACCCCCAGGAC
  P H T E H G A C G L R L E A A P V G V L>
            1810 1820 1830 1840 1850 1860
TGGCCCTGGCTGGCAGAGGTGCATGTGGCTGGTGATCGAGTCTGCACTGGGATCCTCCTG
ACCGGGACCGACCGTCTCCACGTACACCGACCACTAGCTCAGACGTGACCCTAGGAGGAC
  W P W L A E V H V A G D R V C T G I L L>
            1870 1880 1890 1900 1910 1920
GCCCCAGGCTGGGTCCTGGCAGCCACTCACTGTGTCCTCAGGCCAGGCTCTACAACAGTG
CGGGGTCCGACCCAGGACCGTCGGTGAGTGACACAGGAGTCCGGTCCGAGATGTTGTCAC
  A P G W V L A A T H C V L R P G S T T V>
            1930 1940 1950 1960 1970 1980
CCTTACATTGAAGTGTATCTGGGCCGGGCAGGGGCCAGCTCCCTCCCACAGGGCCACCAG
GGAATGTAACTTCACATAGACCCGGCCCGTCCCCGGTCGAGGGAGGGTGTCCCGGTGGTC
  P Y I E V Y L G R A G A S S L P Q G H Q>
            1990 2000 2010 2020 2030 2040
ATGACCTCAGCACCGCCCCTCCTGTGCCAGATGACGGAAGGGTCCTGGATCCTCGTGGGC
TACTGGAGTCGTGGCGGGGAGGACACGGTCTACTGCCTTCCCAGGACCTAGGAGCACCCG
  M T S A P P L L C Q M T E G S W I L V G>
            2050 2060 2070 2080 2090 2100
ATGGCTGTTCAAGGGAGCCGGGAGCTGTTTGCTGCCATTGGTCCTGAAGAGGCCTGGATC
TACCGACAAGTTCCCTCGGCCCTCGACAAACGACGGTAACCAGGACTTCTCCGGACCTAG
  M A V Q G S R E L F A A I G P E E A W I>
            2110 2120 2130 2140 2150 2160
TCCCAGACAGTGGGAGAGGCCAACTTCCTGCCCCCCAGTGGCTCCCCACACTGGCCCACT
AGGGTCTGTCACCCTCTCCGGTTGAAGGACGGGGGGTCACCGAGGGGTGTGACCGGGTGA
  S Q T V G E A N F L P P S G S P H W P T>
            2170 2180 2190 2200 2210 2220
GGAGGCAGCAATCTCTGCCCCCCAGAACTGGCCAAGGCCTCGGGATCCCCGCATGCAGTC
CCTCCGTCGTTAGAGACGGGGGGTCTTGACCGGTTCCGGAGCCCTAGGGGCGTACGTCAG
  G G S N L C P P E L A K A S G S P H A V>
            2230 2240 2250 2260 2270 2280
TACTTCCTGCTCCTGCTGACTCTCCTGATCCAGAGCTGAGGGGCTAGGGTCCCAGCACCA
ATGAAGGACGAGGACGACTGAGAGGACTAGGTCTCGACTCCCCGATCCCAGGGTCGTGGT
  Y F L L L L T L L I Q S *>
            2290
CTTCCCCCTTCTC
GAAGGGGGAAGAG
```

CVSP16 Polypeptide Sequence

Sequence Range: 1 to 753

```
                10 20 30 40 50 60
MARQLLLPLVVLVISPIPGAFQDSALSPTQEEPEDLDCGRPEPSARIVGGSNAQPGTWPW
                70 80 90 100 110 120
QVSLHHGGGHICGGSLIAPSWVLSAAHCFMTNGTLEPAAEWSVLLGvHSQDGPLDGAHTR
              130 140 150 160 170 180
AVAAIVVPANYSQVELGADLALLRLASPASLGPAVWPVCLPRASHRFVHGTACWATGWGD
              190 200 210 220 230 240
VQEADPLPLPWVLQEVELRLLGEATCQCLYSQPGPFNLTLQILPGMLCAGYPGGRRDTCQ
              250 260 270 280 290 300
GDSGGPLVCEEGGRWFQAGITSFGFGCGRRNRPGVFTAVATYEAWIREQVMGSEPGPAFP
              310 320 330 340 350 360
TQPQKTQSDPQEPREENCTIALPECGKAPRPGAWPWEAQVMVPGSRPCHGALVSESWVLA
              370 380 390 400 410 420
PASCFLDPNSSDSPPRDLDAWRVLLPSHPRAERVARLVQHENASWDNAPDLALLQLRTPV
              430 440 450 460 470 480
NLSAASRPVCLPNPEHYFLPGSRCRLARWGRGEPALGPGALLEAELLGGWWCHCLYGRQG
              490 500 510 520 530 540
AAVPLPGDPPHALCPAYQEKEEVGSCWNDSRWSLLCQEEGTWFLAGIRDFPSGCLRPRAF
              550 560 570 580 590 600
FPLQTHGPWISHVTRGAYLEDQLAWDWGPDGEETETQTCPPHTEHGACGLRLEAAPVGVL
              610 620 630 640 650 660
WPWLAEVHVAGDRVCTGILLAPGWVLAATHCVLRPGSTRVPYIEVYLGRAGASSLPQGHQ
              670 680 690 700 710 720
MTSAPPLLCQMTEGSWILVGMAVQGSRELFAAIGPEEAWISQTVGEANFLPPSGSPHWPT
              730 740 750
GGSNLCPPELAKASGSPHAVYFLLLLTLLIQS*
```

### EXAMPLE 2

Expression of the Protease Domains

Nucleic acid encoding each a full length CVSP16 and/or protease domain thereof can be cloned into a derivative of the Pichia pastoris vector pPIC9K (available from Invitrogen; see SEQ ID NO. 13) or pPIC9KX (described below), which is introduced into a suitable Pichia host or other compatible host and used to express the encoded CVSP16 or portion thereof.

Plasmid pPIC9K features include the 5′ AOX1 promoter fragment at 1-948; 5′ AOX1 primer site at 855-875; alpha-factor secretion signal(s) at 949-1218; alpha-factor primer site at 1152-1172; multiple cloning site at 1192-1241; 3′ AOX1 primer site at 1327-1347; 3′ AOX1 transcription termination region at 1253-1586; HIS4 ORF at 4514-1980; kanamycin resistance gene at 5743-4928; 3′ AOX1 fragment at 6122-6879; ColE1 origin at 7961-7288; and the ampicillin resistance gene at 8966-8106. The plasmid pPIC9KX is derived from pPIC9K by eliminating the XhoI site in the kanamycin resistance gene to produce pPIC9KX.

Other vectors that can be used for expression of CVSP16 or portions thereof include, but are not limited to, insect and mammalian vectors as described, for example, above. The protein also can be expressed in E. coli, for example, as inclusion bodies in the cytoplasm or in the cytoplasm using the strain Origami (i.e., Origami B from Novagen, Madison Wis.) that permits folding in the cytoplasm. CVSP16 also can be expressed in the periplasmic space.

### EXAMPLE 3

Assays for Identification of Candidate Compounds that Modulate the Activity of a Serine Protease

Assay for Identifying Inhibitors

The ability of test compounds to act as inhibitors of catalytic activity of a catalytic activity of a CVSP16 polypeptide can be assessed in an amidolytic assay. Compound mediated inhibition of amidolytic activity of a CVSP16 polyeptide or a protease domain portion thereof, can be measured by IC50 values in such an assay.

An exemplary assay buffer is HBSA (10 mM Hepes, 150 mM sodium chloride, pH 7.4, 0.1% bovine serum albumin). All reagents can be purchased from Sigma Chemical Co. (St. Louis, Mo.), unless otherwise indicated. Two IC50 assays at 30-minute (a 30-minute preincubation of test compound and enzyme) and at 0-minutes (no preincubation of test compound and enzyme) are conducted. For the IC50 assay at 30-minute, the following reagents are combined in appropriate wells of a Corning microtiter plate: 50 microliters of HBSA, 50 microliters of the test compound, diluted (covering a broad concentration range) in HBSA (or HBSA alone for the uninhibited velocity measurement), and 50 microliters of the SP or protease domain thereof diluted in buffer, yielding a final enzyme concentration of about 0.5-5 nM. Following a 30-minute incubation at ambient temperature, the assay is initiated by the addition of 50 microliters of a substrate for the particular SP (see, e.g., table and discussion below), which was reconstituted in deionized water, and diluted in HBSA prior to the assay, yielding a final volume of 200 microliters and a final substrate concentration of 200-600 μM.

For an IC50 assay at 0-minute, the same reagents are combined: 50 microliters of HBSA, 50 microliters of the test compound, diluted (covering the identical concentration range) in HBSA (or HBSA alone for uninhibited velocity measurement), and 50 microliters of the substrate, such as a chromogenic substrate. The assay is initiated by the addition of 50 microliters of SP. The final concentrations of all components are identical in both IC50 assays (at 30- and 0-minute incubations).

The initial velocity of the substrate hydrolysis is measured in both assays by, for example for a chromogenic substrate, the change in absorbance at a particular wavelength, using a Thermo Max® Kinetic Microplate Reader (Molecular Devices) over a 5 minute period, in which less than 5% of the added substrate was hydrolyzed. The concentration of added inhibitor, which caused a 50% decrease in the initial rate of hydrolysis was defined as the respective IC50 value in each of the two assays (30-and 0-minute).

Another Assay for Identifying Inhibitors

Test compounds for inhibition of the protease activity of the protease domain are assayed in Costar 96 well tissue culture plates (Corning N.Y.). Approximately 0.5-5 nM of the CVSP16 or protease domain thereof is mixed with varying concentrations of inhibitor in 29.2 mM Tris, pH 8.4, 29.2 mM imidazole, 217 mM NaCl (100 mL final volume) and allowed to incubate at room temperature for 30 minutes. About 200-600 μM substrate is added, and the reaction monitored in a SpectraMAX® Plus microplate reader (Molecular Devices, Sunnyvale Calif.) by following the change in a parameter correlated with hydrolysis, such as absorbance for a chromogenic substrate for 1 hour at 37° C.

Alternative Assay for Screening CVSP16

The protease domain of CVSP16 or full-length polypeptide or other catalytically active portion thereof is expressed in Pichia pastoris. Test compounds are screened for modulation of the activity of the CVSP16 polypeptide or portion thereof. Approximately 1-20 nM CVSP16 is mixed in Costar 96 well tissue culture plates (Corning N.Y.) with varying concentrations of test compounds and/or known inhibitors or agonsists in 29.2 mM Tris, pH 8.4, 29.2 mM Imidazole, 217 mM NaCl (100 μL final volume), and allowed to incubate at room temperature for 30 minutes. 200-600 μM of a chromogenic substrate is added, and the reaction is monitored in a SpectraMAX Plus microplate reader (Molecular Devices, Sunnyvale Calif.) by measuring the change in absorbance at 405 nm for 30 minutes at 37° C.

Identification of Substrates

Particular substrates for use in the assays can be identified empirically by testing substrates. The following list of substrates are exemplary of those that can be tested.

| Substrate name | Structure |
|---|---|
| S 2366 | pyroGlu-Pro-Arg-pNA.HCl |
| spectrozyme t-PA | CH₃SO₂-D-HHT-Gly- |
| | Arg-pNA.AcOH |
| N-p-tosyl-Gly-Pro-Arg-pNA | N-p-tosyl-Gly-Pro-Arg-pNA |
| Benzoyl-Val-Gly-Arg-pNA | Benzoyl-Val-Gly-Arg-pNA |
| Pefachrome t-PA | CH₃SO₂-D-HHT-Gly-Arg-pNA |
| S 2765 | N-α-Z-D-Arg-Gly-Arg-pNA.2HCl |
| S 2444 | pyroGlu-Gly-Arg-pNA.HCl |
| S 2288 | H-D-Ile-Pro-Arg-pNA.2HCl |
| spectrozyme UK | Cbo-L-(γ)Glu(α-t-BuO)- |
| | Gly-Arg-pNA.2AcOH |
| S 2302 | H-D-Pro-Phe-Arg-pNA.2HCl |
| S 2266 | H-D-Val-Leu-Arg-pNA.2HCl |
| S 2222 | Bz-Ile-Glu(g-OR)-Gly-Arg-pNA.HCl |
| | R = H(50%) and R = CH₃(50%) |
| Chromozyme PK | Benzoyl-Pro-Phe-Arg-pNA |
| S 2238 | H-D-Phe-Pip-Arg-pNA.2HCl |
| S 2251 | H-D-Val-Leu-Lys-pNA.2HCl |
| Spectrozyme PI | H-D-Nle-HHT-Lys-pNA.2AcOH |
| | Pyr-Arg-Thr-Lys-Arg-AMC |
| | H-Arg-Gln-Arg-Arg-AMC |
| | Boc-Gln-Gly-Arg-AMC |
| | Z-Arg-Arg-AMC |
| Spectrozyme THE | H-D-HHT-Ala-Arg-pNA.2AcOH |
| Spectrozyme fXIIa | H-D-CHT-Gly-Arg-pNA.2AcOH |
| | CVS 2081-6 (MeSO₂-dPhe- |
| | Pro-Arg-pNA) |
| | Pefachrome fVIIa (CH₃SO₂-D-CHA- |
| | But-Arg-pNA) |

pNA = para-nitranilide (chromogenic)
AMC = amino methyl coumarin (fluorescent)

If none of the above substrates are cleaved, a coupled assay can be used. Briefly, such assays test the ability of the protease to activate an enzyme, such as plasminogen and trypsinogen. To perform these assays, the single chain protease is incubated with a zymogen, such as plasminogen or trypsinogen, in the presence of a known substrate for plasmin or trypsin, such as a Spectrozyme substrate. If a single chain CVSP16 activates the zymogen, the activated enzyme, such as plasmin and trypsin, will degrade the substrate therefor.

### EXAMPLE 4

Other Assays

These assays are described with reference to MTSP1, but such assays can be readily adapted for use with CVSP16.

Amidolytic Assay for Determining Inhibition of Serine Protease Activity of Matriptase or MTSP1

The ability of test compounds to act as inhibitors of rMAP catalytic activity was assessed by determining the inhibitor-induced inhibition of amidolytic activity by the MAP, as measured by IC₅₀ values. The assay buffer was HBSA (10 mM Hepes, 150 mM sodium chloride, pH 7.4, 0.1% bovine serum albumin). All reagents were from Sigma Chemical Co. (St. Louis, Mo.), unless otherwise indicated.

Two IC₅₀ assays (a) one at either 30-minutes or 60-minutes (a 30-minute or a 60-minute preincubation of test compound and enzyme) and (b) one at 0-minutes (no preincubation of test compound and enzyme) were conducted. For the IC₅₀ assay at either 30-minutes or 60-minutes, the following reagents were combined in appropriate wells of a Corning microtiter plate: 50 microliters of HBSA, 50 microliters of the test compound, diluted (covering a broad concentration range) in HBSA (or HBSA alone for uninhibited velocity measurement), and 50 microliters of the rMAP (Corvas International) diluted in buffer, yielding a final enzyme concentration of 250 pM as determined by active site titration. Following either a 30-minute or a 60-minute incubation at ambient temperature, the assay was initiated by the addition of 50 microliters of the substrate S-2765 (N-α-Benzyloxycarbonyl-D-arginyl-L-glycyl-L-arginine-p-nitroaniline dihydrochloride; DiaPharma Group, Inc.; Franklin, Ohio) to each well, yielding a final assay volume of 200 microliters and a final substrate concentration of 100 μM (about 4-times Kₘ). Before addition to the assay mixture, S-2765 was reconstituted in deionized water and diluted in HBSA. For the IC₅₀ assay at 0 minutes; the same reagents were combined: 50 microliters of HBSA, 50 microliters of the test compound, diluted (covering the identical concentration range) in HBSA (or HBSA alone for uninhibited velocity measurement), and 50 microliters of the substrate S-2765. The assay was initiated by the addition of 50 microliters of rMAP. The final concentrations of all components were identical in both IC₅₀ assays (at 30- or 60- and 0-minute).

The initial velocity of chromogenic substrate hydrolysis was measured in both assays by the change of absorbance at 405 nM using a Thermo Max® Kinetic Microplate Reader (Molecular Devices) over a 5 minute period, in which less than 5% of the added substrate was used. The concentration of added inhibitor, which caused a 50% decrease in the initial rate of hydrolysis was defined as the respective IC₅₀ value in each of the two assays (30- or 60-minutes and 0-minute).

### In Vitro Enzyme Assays for Specificity Determination

The ability of compounds to act as a selective inhibitor of matriptase activity was assessed by determining the concentration of test compound that inhibits the activity of matriptase by 50%, (IC₅₀) as described in the above Example, and comparing IC₅₀ value for matriptase to that determined for all or some of the following serine proteases: thrombin, recombinant tissue plasminogen activator (rt-PA), plasmin, activated protein C, chymotrypsin and factor Xa.

The buffer used for all assays was HBSA (10 mM HEPES, pH 7.5, 150 mM sodium chloride, 0.1% bovine serum albumin). The assay for IC₅₀ determinations was conducted by combining in appropriate wells of a Corning microtiter plate, 50 microliters of HBSA, 50 microliters of the test compound at a specified concentration (covering a broad concentration range) diluted in HBSA (or HBSA alone for V₀ (uninhibited velocity) measurement), and 50 microliters of the enzyme diluted in HBSA. Following a 30 minute incubation at ambient temperature, 50 microliters of the substrate at the concentrations specified below were added to the wells, yielding a final total volume of 200 microliters. The initial velocity of chromogenic substrate hydrolysis was measured by the change in absorbance at 405 nm using a Thermo Max® Kinetic Microplate Reader over a 5 minute period in which less than 5% of the added substrate was used. The concentration of added inhibitor which caused a 50% decrease in the initial rate of hydrolysis was defined as the IC₅₀ value.

Thrombin (fIIa) Assay

Enzyme activity was determined using the chromogenic substrate, Pefachrome t-PA (CH₃SO₂-D-hexahydrotyrosine-glycyl-L-Arginine-p-nitroaniline, obtained from Pentapharm Ltd.). The substrate was reconstituted in deionized water prior to use. Purified human α-thrombin was obtained from Enzyme Research Laboratories, Inc. The buffer used for all assays was HBSA (10 mM HEPES, pH 7.5, 150 mM sodium chloride, 0.1% bovine serum albumin).

IC₅₀ determinations were conducted where HBSA (50 μL), α-thrombin (50 μl) (the final enzyme concentration is 0.5 nM) and inhibitor (50 μl) (covering a broad concentration range), were combined in appropriate wells and incubated for 0 or 30 minutes at room temperature prior to the addition of substrate Pefachrome-t-PA (50 μl) (the final substrate concentration is 250 μM, about 5 times Kₘ). The initial velocity of Pefachrome t-PA hydrolysis was measured by the change in absorbance at 405 nm using a Thermo Max® Kinetic Microplate Reader over a 5 minute period in which less than 5% of the added substrate was used. The concentration of added inhibitor which caused a 50% decrease in the initial rate of hydrolysis was defined as the IC₅₀ value.

Factor Xa

Factor Xa catalytic activity was determined using the chromogenic substrate S-2765 (N-benzyloxycarbonyl-D-arginine-L-glycine-L-arginine-p-nitroaniline), obtained from DiaPharma Group (Franklin, Ohio). All substrates were reconstituted in deionized water prior to use. The final concentration of S-2765 was 250 μM (about 5-times Km). Purified human Factor X was obtained from Enzyme Research Laboratories, Inc. (South Bend, Ind.) and Factor Xa (FXa) was activated and prepared from it as described (Bock, P. E., Craig, P. A., Olson, S. T., and Singh, P. Arch. Biochem. Biophys. 273:375-388 (1989)) The enzyme was diluted into HBSA prior to the assay were the final concentration was 0.25 nM.

Recombinant Tissue Plasminogen Activator (rt-PA) Assay

rt-PA catalytic activity was determined using the substrate, Pefachrome t-PA (CH₃SO₂-D-hexahydrotyrosine-glycyl-L-arginine-p-nitro-aniline, obtained from Pentapharm Ltd.). The substrate was made up in deionized water followed by dilution in HBSA prior to the assay where the final concentration was 500 micromolar (about 3-times Km). Human rt-PA (Activase®) was obtained from Genentech Inc. The enzyme was reconstituted in deionized water and diluted into HBSA prior to the assay where the final concentration was 1.0 nM.

Plasmin Assay

Plasmin catalytic activity was determined using the chromogenic substrate, S-2366 [L-pyroglutamyl-L-prolyl-L-arginine-p-nitroaniline hydrochloride], which was obtained from DiaPharma group. The substrate was made up in deionized water followed by dilution in HBSA prior to the assay in which the final concentration was 300 micromolar (about 2.5-times Km). Purified human plasmin was obtained from Enzyme Research Laboratories, Inc. The enzyme was diluted into HBSA prior to the assay where final concentration was 1.0 nM.

Activated Protein C (aPC) Assay

aPC catalytic activity was determined using the chromogenic substrate, Pefachrome PC (delta-carbobenzloxy-D-lysine-L-prolyl-L-arginine-p-nitroaniline dihydrochloride), obtained from Pentapharm Ltd.). The substrate was made up in deionized water followed by dilution in HBSA prior to the assay where the final concentration was 400 micromolar (about 3-times Km). Purified human aPC was obtained from Hematologic Technologies, Inc. The enzyme was diluted into HBSA prior to the assay where the final concentration was 1.0 nM.

Chymotrypsin Assay

Chymotrypsin catalytic activity was determined using the chromogenic substrate, S-2586 (methoxy-succinyl-L-arginine-L-prolyl-L-tyrosyl-p-nitroanilide), which was obtained from DiaPharma Group. The substrate was made up in deionized water followed by dilution in HBSA prior to the assay where the final concentration was 100 micromolar (about 9-times Km). Purified (3×-crystallized; CDI) bovine pancreatic alpha-chymotrypsin was obtained from Worthington Biochemical Corp. The enzyme was reconstituted in deionized water and diluted into HBSA prior to the assay where the final concentration was 0.5 nM.

Since modifications will be apparent to those of skill in this art, it is intended that this invention be limited only by the scope of the appended claims.

1. A substantially purified single chain or multi-chain polypeptide, selected from:
a) a polypeptide, comprising a protease domain of a serine protease 16 (CVSP16) or a functionally active portion thereof or a domain thereof, wherein: if the polypeptide includes residues that correspond to Gln₆₆₀ and Met₆₆₁, it does not include at least 5 contiguous amino acids from SEQ ID No. 21 inserted between residues that correspond to Gln₆₆₀ and M₆₆₁ of SEQ ID No. 21; or
b) a substantially purified single chain or multi-chain polypeptide, comprising at least two protease domains of a serine protease 16 (CVSP16), wherein the polypeptide comprises at least 5 contiguous amino acids corresponding to residues 508-544 of SEQ ID No. 6. or comprises the contiguous sequence Asn Asp Ser or Trp Asn Asp or Ser Cys Trp Asn Asp Ser or Cys Trp Asn Asp Ser or Gln Thr His or Leu Gln Thr His in the second protease domain.

2. The polypeptide of claim 1, wherein one protease domain comprises amino acids 323-550 or 326-550 of SEQ ID No. 6. or has at least about 60%, 70%, 80%, 90% or 95% sequence identity to amino acids 326-550 of SEQ ID No. 6.

3. The polypeptide of claim 1, wherein one protease domain comprises amino acids 46-286 of SEQ ID No. 6 or has at least about 60%, 70%, 80%, 90% or 95% sequence identity to amino acids 46-286 of SEQ ID No. 6.

4. A polypeptide of claim 1 that contains two or three chains.

5. A substantially purified single chain or multi-chain polypeptide of claim 1, comprising a protease domain of a serine protease 16 (CVSP16) or a catalytically active portion thereof or a domain thereof, wherein the polypeptide does not include at least 5 contiguous amino acids from SEQ ID No. 21; and the polypeptide contains one, two or three chains.

6. A polypeptide of claim 1 that comprises one protease domain.

7. A polypeptide of claim 1 that comprises two protease domains.

8. A polypeptide of claim 1 or claim 5, wherein a protease domain comprises amino acids 46-286 or 326-550 of SEQ ID No. 6 or amino acids that share at least about 60%, 70%, 80%, 90% or 95% homology to amino acids 46-286 or 326-550 of SEQ ID No. 6.

9. A polypeptide of claim 1 or claim 5, wherein the contiguous sequence is not present in the polypeptide at any locus.

10. A polypeptide of claim 1 or claim 5, wherein the polypeptide comprises the contiguous sequence Gly His Gln Met Thr Ser (SEQ ID No. 6, amino acids 658-663).

11. A polypeptide of claim 7, that comprises amino acids 46-286 and 326-550 of SEQ ID No. 6 or amino acids that share at least about 60%, 70%, 80%, 90% or 95% sequence identity to each of amino acids 46-286 and amino acids 326-550 of SEQ ID No. 6.

12. A polypeptide of claim 1 or claim 5, wherein the CVSP16 portion of the polypeptide consists essentially of amino acids 46-286 of SEQ ID No. 6.

13. A polypeptide of claim 1 or claim 5, wherein the CVSP16 portion consists essentially of amino acids 323-550 or 326-550 of SEQ ID No. 6.

14. A polypeptide of claim 12, wherein the CVSP16 portion of the polypeptide has at least about 60%, 70%, 80%, 90% or 95% sequence identity to amino acids 46-286 of SEQ ID No. 6.

15. A polypeptide of claim 13, wherein the CVSP16 portion of the polypeptide has at least about 60%, 70%, 80%, 90% or 95% sequence identity to amino acids 46-286 of SEQ ID No. 6.

16. A polypeptide of claim 5, wherein the CVSP16 portion of the polypeptide consists essentially of amino acids 46-550 of SEQ ID No. 6.

17. The polypeptide of claim 5, wherein:
the CVSP16 portion of the polypeptide consists essentially of a protease domain of a CVSP16 or a catalytically active portion thereof.

18. A polypeptide of claim 1, comprising the sequence of amino acids set forth in SEQ ID No. 6 or set forth as amino acids 24-752 in SEQ ID No. 6.

19. A polypeptide of claim 5, consisting essentially of the sequence of amino acids set forth in SEQ ID No. 6 or consisting essentially of amino acids 24-752 of SEQ ID No. 6.

20. A polypeptide of claim 5, wherein the CVSP16 is a human protein.

21. A polypeptide of claim 1 that has catalytic activity.

22. A polypeptide of claim 5, wherein the level of expression and/or activity of the CVSP16 in tumor cells differs from its level of expression and/or activity in non-tumor cells.

23. A polypeptide of claim 5, wherein the CVSP16 polypeptide is detectable in a body fluid at a level that differs from its level in body fluids in a subject not having a tumor.

24. A polypeptide of claim 1 or claim 5 that is a single chain.

25. A polypeptide of claim 5 that is a two or three chain polypeptide.

26. A polypeptide of claim 1, wherein:
the CVSP16 is present in a tumor; and
a substrate or cofactor for the CVSP16 is expressed at levels that differ from its level of expression in a non-tumor cell in the same type of tissue.

27. A polypeptide of claim 1 that has at least about 60%, 80%, 90% or 95% sequence identity with a polypeptide that comprises the sequence of amino acids set forth as SEQ ID No. 6 or a catalytically active portion thereof.

28. A polypeptide of claim 1 that comprises a protease domain encoded by a nucleic acid molecule selected from the group consisting of:
a) a nucleic acid molecule that hybridizes under conditions of high stringency along at least 70% of its full length to a nucleic acid molecule comprising a sequence of nucleotides set forth in SEQ ID No. 5 that encodes amino acids 46 to 285 or 326 to 550 of SEQ ID No. 6;
b) a nucleic acid molecule, comprising the sequence of nucleotides set forth in SEQ ID No. 5 that encodes resides 24-752; and
c) a nucleic acid molecule that comprises degenerate codons of a) or b).

29. A polypeptide of claim 5 that is selected from the group consisting of:
a polypeptide encoded by the sequence of nucleotides set forth in SEQ ID No. 5 or a catalytically active portion or ligand or substrate binding portion of the polypeptide;
a polypeptide encoded by a sequence of nucleotides that hybridizes under conditions of high stringency along 70% of its full length to the sequence of nucleotides set forth in SEQ ID No. 5 or to a sequence of nucleotides comprising degenerate codons thereof;
a polypeptide that comprises a sequence of amino acids having at least about 85%, 86%, 88%, 90%, 93% or 95% sequence identity with the sequence of amino acids set forth in SEQ ID No. 6; and
a polypeptide encoded by a splice variant of the sequence of nucleotides set forth in SEQ ID No. 5.

30. A polypeptide that is a mutein of the polypeptide of claim 1 or claim 5, wherein:
up to about 50% of the amino acids are replaced with another amino acid; and
the resulting polypeptide is a single chain, two-chain or three-chain polypeptide that has catalytic activity of at least 1% of the unmutated polypeptide.

31. A polypeptide of claim 30, wherein up to about 25% of the amino acids are replaced with another amino acid.

32. A polypeptide of claim 30, wherein up to about 10% of the amino acids are replaced with another amino acid.

33. A polypeptide of claim 30, wherein the resulting polypeptide is a single chain or two-chain or three-chain polypeptide and has catalytic activity of at least 10% of the unmutated polypeptide.

34. A polypeptide of claim 32, wherein the resulting polypeptide is a single chain or two-chain or three-chain polypeptide and has catalytic activity of at least 10% of the unmutated polypeptide.

35. A polypeptide of claim 30, wherein up to about 95% of the amino acids are conserved or are replaced by conservative amino acid substitutions.

36. A polypeptide of claim 1 or claim 5, wherein an unpaired Cysteine in a protease domain is replaced with another amino acid.

37. The polypeptide of claim 36, wherein the replacing amino acid is a serine.

38. A polypeptide of claim 36, wherein the unpaired Cys in a protease domain is amino acid C₁₅₉ and/or C₄₃₀.

39. A nucleic acid molecule, comprising a sequence of nucleotides that encodes a polypeptide of claim 1 or claim 5.

40. A plasmid or vector comprising the nucleic acid molecule of claim 39.

41. A vector of claim 40 that is an expression vector.

42. A vector of claim 41 that includes a sequence of nucleotides that directs secretion of any protein encoded by a sequence of nucleotides operatively linked thereto.

43. A vector of claim 41 that is a Pichia vector, a baculovirus vector, an mammalian cell vector or an E. coli vector.

44. A cell, comprising a plasmid or vector of claim 40.

45. The cell of claim 44 that is a prokaryotic cell.

46. The cells of claim 44 that is a eukaryotic cell.

47. The cell of claim 44 that is selected from among a bacterial cell, a yeast cell, a plant cell, an insect cell and an animal cell.

48. The cell of claim 47 that is a mammalian cell.

49. A method for producing a polypeptide that contains a protease domain of a CVSP16, comprising:
culturing a cell of claim 44 under conditions whereby the encoded protein is expressed by the cell; and
recovering the expressed protein.

50. The method of claim 49, wherein the cell is a Pichia cell and the protein is optionally secreted into the culture medium or the cell is a mammalian cell.

51. The method of claim 49, wherein the polypeptide is secreted into the culture medium.

52. The method of claim 49, wherein the polypeptide is expressed in the cytoplasm of the host cell.

53. The method of claim 49, wherein the polypeptide is expressed in inclusion bodies, and the method further comprises isolating the polypeptide from the inclusion bodies under conditions, whereby the polypeptide refolds into a proteolytically active form.

54. An antisense nucleic acid molecule that:
comprises at least 14 and less than about 150 contiguous nucleotides or modified nucleotides that are complementary to a contiguous sequence of nucleotides of a CVSP16 of claim 5;
comprises at least 16 and less than about 150 contiguous nucleotides or modified nucleotides that are complementary to a contiguous sequence of nucleotides of a CVSP16 of claim 5;
comprises at least 30 and less than about 150 contiguous nucleotides or modified nucleotides that are complementary to a contiguous sequence of nucleotides of a CVSP16 of claim 5,
wherein the contiguous nucleotides span nucleotides corresponding to nucleotides 1978-1983 of SEQ ID No. 5.

55. A double-stranded RNA (dsRNA) molecule that comprises at least about 21 contiguous nucleotides or modified nucleotides that are complementary to all or a portion of a contiguous sequence of nucleotides that encodes the sequence of amino set forth as SEQ ID No. 6.

56. The dsRNA of claim 55, wherein the contiguous nucleotides span nucleotides corresponding to nucleotides 1978-1983 of SEQ ID No. 5.

57. An antibody that binds to a polypeptide of claim 5 with at least 10-fold greater affinity than to a polypeptide that includes the at least 5 contiguous amino acids set forth in SEQ ID No. 21.

58. An antibody that binds to a polypeptide of claim 5 with at least 2-fold greater affinity than to a polypeptide that includes the at least 5 contiguous amino acids set forth in SEQ ID No. 21.

59. The antibody of claim 58, wherein the contiguous sequence is inserted between amino acids corresponding to Q660 and M661 of a CVSP16 polypeptide that comprises amino acids 24-752 of SEQ ID No. 6.

60. An antibody of claim 57 that binds with at least 100-fold greater affinity.

61. An antibody of claim 58 that inhibits a catalytic activity of the polypeptide.

62. An antibody of claim 58 that inhibits a ligand or substrate binding activity of the polypeptide.

63. An antibody that specifically binds to a single-chain form of a protease domain 1 (PD1) of a CVSP16 polypeptide or to a single-chain form of a protease domain 2 (PD2) of a CVSP16 polypeptide.

64. An antibody that specifically binds to a single-chain form of a CVSP16 polypeptide of claim 5.

65. A conjugate, comprising:
a) a CVSP16 polypeptide; and
b) a targeting agent linked to the protein directly or via a linker.

66. A combination, comprising:
a) a modulator of the catalytic activity or substrate or ligand binding activity of a CVSP16 polypeptide; and
b) another treatment agent or agent selected from anti-tumor and anti-angiogenic treatments or agents.

67. The combination of claim 66, wherein the modulator is an inhibitor.

68. The combination of claim 67, wherein the inhibited activity is catalytic activity.

69. The combination of claim 68, wherein the modulator inhibitor and the anti-tumor and/or anti-angiogenic agent are formulated in a single pharmaceutical composition or each is formulated in separate pharmaceutical compositions.

70. The combination of claim 69, wherein the inhibitor is selected from antibodies and antisense oligonucleotides.

71. A solid support comprising two or more CVSP16 polypeptides of claim 5 linked thereto either directly or via a linker.

72. The support of claim 71, wherein the polypeptides comprise an array.

73. The support of claim 71, further comprising a plurality of different serine protease domains linked to the support directly or via a linker.

74. A method for identifying compounds that modulate the protease activity of a CVSP16 polypeptide, comprising:
contacting a polypeptide of claim 5 with a substrate proteolytically cleaved by the CVSP16 polypeptide, and, either simultaneously, before or after, adding a test compound or plurality thereof;
measuring the amount of substrate cleaved in the presence of the test compound; and
selecting compounds that change the amount cleaved compared to a control, whereby compounds that modulate an activity of the CVSP16 are identified.

75. The method of claim 74, wherein the test compounds are small molecules, peptides, peptidomimetics, natural products, antibodies or fragments thereof.

76. The method of claim 74, wherein a plurality of the test substances are screened simultaneously.

77. The method of claim 74, wherein the change in the amount of substrate cleaved is assessed by comparing the amount cleaved in the presence of the test compound with the amount cleaved in the absence of the test compound.

78. The method of claim 74, wherein the polypeptides comprise an array.

79. The method of claim 74, wherein the polypeptides comprise a plurality of different serine proteases.

80. A method of identifying a compound that specifically binds to a form of a CVSP16, comprising:
contacting a CVSP16 polypeptide of claim 5, or a functionally active portion thereof, with a test compound or plurality thereof under conditions conducive to binding thereof, and either:
a) identifying test compounds that specifically bind to a form, or to a functionally active portion thereof; or
b) identifying test compounds that inhibit binding of a compound known to bind to a form of the polypeptide or to a functionally active portion thereof, wherein:
the known compound is contacted with the polypeptide either before, simultaneously with, or after the test compound;
a functionally active portion is a proteolytically active portion and/or a substrate or ligand binding portion; and
a form is one or more of a single chain form, a two-chain form, a three chain form and/or a four chain form and the form is activated or is a zymogen or includes one or more activated domains.

81. The method of claim 80, wherein the polypeptide is linked either directly, or indirectly, via a linker, to a solid support.

82. The method of claim 80, wherein the test compounds are small molecules, peptides, peptidomimetics, natural products, antibodies or fragments thereof.

83. The method of claim 80, wherein a plurality of the test substances are screened simultaneously.

84. The method of claim 83, wherein a plurality of the polypeptides are linked to a solid support.

85. A method for identifying activators of a zymogen form of a CVSP16 or functionally active portion thereof, comprising:
contacting a zymogen form of a CVSP16 polypeptide of claim 5, or a functionally active portion thereof, with a substrate of the activated form of the polypeptide;
adding a test compound, wherein the test compound is added before, after, or simultaneously with, the addition of the substrate; and
detecting cleavage of the substrate, thereby identifying compounds that activate the zymogen, wherein:
a functionally active portion is a proteolytically active portion and/or a substrate or ligand binding portion; and
a zymogen is one or more of a single chain form, a two-chain form or a three chain form that includes at least one domain that is not activated.

86. The method of claim 85, wherein the substrate is a chromogenic or fluorogenic substrate.

87. The method of claim 85, wherein the test compounds are small molecules, peptides, peptidomimetics, natural products, antibodies or fragments thereof.

88. A method for treating or preventing a neoplastic disease in a mammal, comprising administering to a mammal an effective amount of a modulator of a polypeptide of claim 5.

89. The method of claim 88, wherein the modulator is an inhibitor.

90. The method of claim 88, wherein the modulator is an antibody that specifically binds to the polypeptide, or a fragment or derivative of the antibody containing a binding domain thereof, wherein the antibody is a polyclonal antibody or a monoclonal antibody.

91. A method of inhibiting tumor initiation, growth, progression, or treatment of a malignant or pre-malignant condition, comprising administering an agent that modulates activation cleavage of the zymogen form of a CVSP16 polypeptide of claim 5 or a potentially functionally active portion thereof, or inhibits an activity of the activated form of CVSP16, or a proteolytically active portion thereof, wherein a functionally active portion is a proteolytically active portion and/or a substrate or ligand binding portion.

92. The method claim 91, wherein the agent inhibits cleavage.

93. The method of claim 91, wherein the condition is a tumor or cancer of the uterus, breast, colon, lung, kidney, rectum, prostate, cervix, testes, stomach, esophagus, ovary, or small intestine, or is a leukemia or a lymphoma.

94. The method of claim 91, wherein the agent is an antisense oligonucleotide, double-stranded RNA (dsRNA) or an antibody.

95. The method of claim 91, further comprising administering another treatment or agent selected from anti-tumor and anti-angiogenic treatments or agents.

96. A method of identifying a compound that binds to one or more forms of a CVSP16 polypeptide of claim 5, and/or to a functionally active portion thereof comprising:
contacting a test compound with two or more forms of a CVSP16 polypeptide of claim 5;
and determining to which form or forms the compound binds; and
if it binds to a form of a CVSP16 polypeptide, further
determining whether the compound has at least one of the following properties:
(i) inhibits activation cleavage of a zymogen form of polypeptide;
(ii) inhibits activity of a form; and
(iii) inhibits dimerization of the polypeptide, wherein:
a functionally active portion is a catalytically active portion and/or a substrate or ligand binding portion; and
a form is one or more of a single chain form, a two-chain form, a three chain form and/or a four chain form and the form is activated or is a zymogen or includes one or more activated domains.

97. A method of detecting neoplastic disease, comprising: detecting a polypeptide that comprises a polypeptide of claim 5 in a biological sample, wherein the amount, form, and/or activity detected differs from the amount, form, and/or activity of the polypeptide detected from a subject without neoplastic disease.

98. The method of claim 97, wherein the biological sample is selected from the group consisting of blood, urine, saliva, tears, synovial fluid, sweat, interstitial fluid, sperm, cerebrospinal fluid, ascites fluid, and/or tumor tissue biopsy and circulating tumor cells.

99. The method of claim 96, wherein the biological sample is selected from the group consisting of blood, urine, saliva, tears, synovial fluid, sweat, interstitial fluid, cerebrospinal fluid, semen, ascites fluid, tumor tissue biopsy and circulating tumor cells.

100. The method of claim 97, wherein the one or more forms of the CVSP16 polypeptide consist(s) essentially of a protease domain.

101. A method of diagnosing the presence of a pre-malignant lesion, malignancy, or other pathologic condition in a subject, comprising:
obtaining a biological sample from the subject;
exposing it to an agent that binds to one or more forms of a CVSP16 polypeptide or inhibits or potentiates an activity of the polypeptide; and
detecting binding and/or a change in the activity, wherein:
the pathological condition is characterized by the presence, excess or absence of a three-chain, two-chain and/or single-chain form; and
detection of binding and/or a change in the activity is indicative of the pathological condition in the subject.

102. The method of claim 101, wherein an activity is inhibited.

103. The method of claim 101, wherein the agent is an antibody that specifically binds to a CVSP16 polypeptide.

104. The method of claim 101, wherein the sample is bodily fluid selected from blood, urine, sweat, saliva, cerebrospinal fluid or synovial fluid.

105. A method of monitoring tumor progression and/or therapeutic efficacy, comprising detecting and/or quantifying the level, form, and/or activity of a CVSP16 polypeptide in a bodily tissue or fluid sample.

106. The method of claim 105, wherein the tumor is a tumor of the uterus, breast, colon, lung, kidney, rectum, prostate, cervix, testes, stomach, esophagus, ovary, or small intestine, or is a leukemia or a lymphoma

107. The method of claim 105, wherein the bodily fluid is blood, urine, sweat, saliva, cerebrospinal fluid or synovial fluid.

108. A method of inhibiting tumor invasion or metastasis or treating a malignant or pre-malignant condition, comprising administering an agent that inhibits activation of the zymogen form of CVSP16 or an activity of an activated form.

109. The method of claim 108, wherein the condition is a condition of the uterus, breast, colon, lung, kidney, rectum, prostate, cervix, testes, stomach, esophagus, ovary, or small intestine, or is a leukemia or a lymphoma.

110. The method of claim 108, further comprising administering another treatment or agent selected from anti-tumor and anti-angiogenic treatments or agents.

111. The method of claim 108, wherein the agent is an antisense oligonucleotide or an antibody.

112. A signal sequence, consisting essentially of amino acids 1-23 of SEQ ID No. 6.

113. A pro-polypeptide, comprising the signal sequence of claim 107, wherein the signal sequence is heterologous to a polypeptide operatively linked thereto.

114. A polypeptide, comprising a portion of a CVSP16 poly-peptide, wherein the portion consists essentially of amino acids 1-23 of SEQ ID No. 6.

115. A computational method for screening compounds, comprising:
assessing the interaction of a test compound with a computer-simulated polypeptide that has the sequence of amino acids of a polypeptide of any of claims 1, 4 and 6; and
identifying test compounds that interact with the polypeptide, wherein assessment is effected in silico.

116. A recombinant non-human animal, wherein an endogenous gene that encodes a polypeptide of claim 4 has been deleted or inactivated by homologous recombination or insertional mutagenesis of the animal or an ancestor thereof.

117. A transgenic non-human, comprising heterologous nucleic acid that encodes a polypeptide of claim 4.

118. The conjugate of claim 65, wherein the targeting agent permits
i) affinity isolation or purification of the conjugate;
ii) attachment of the conjugate to a surface;
iii) detection of the conjugate; or
iv) targeted delivery to a selected tissue or cell.

119. The polypeptide of claim 1, wherein a protease domain comprises amino acids 47-286 of SEQ ID No. 6 or amino acids that share at least about 60%, 70%, 80%, 90% or 95% homology to amino acids 47-286 of SEQ ID No. 6.

120. The polypeptide of claim 1, wherein the CVSP16 portion of the polypeptide consists essentially of amino acids 47-286 of SEQ ID No. 6.

121. The polypeptide of claim 5, wherein the CVSP16 portion of the polypeptide consists essentially of amino acids 47-550 of SEQ ID No. 6.
